SEQUENCE LISTING (2 sequences)

SEQ ID NO: 1 - DNA, 143 bp, Homo sapiens
  misc_feature (1)...(143): n = A,T,C or G
ccacgcggtggcggccgcacactcgtccacatccacacaggcgccctcgtccagcacccagccnactttacactcgccgcagtctntgntggtcaggcccgagcacgttttgcaggactcgttacaggntgtgcagatgctgt

SEQ ID NO: 2 - DNA, 441 bp, Homo sapiens
aggtacactcatcctgcgtatcatcactgccatgtcctgataccccagctctgccatattgcccttcttttttgcggtatgatgaccacatagaggcccaacctcttaaacacatcaataccaatgatcacatttcaatctagacttctaagcaacggctgaaatctctccaggccaaaggagagtttgtatcaccttaccagaagcttctccggaacaattggccagaagcctagagttcagaaacccagacacatgcagtaagcaatttccagtttctctataatttagaagaggacaccatgatatgtaatgcggggtctgggaggttggaatgcctccataaaacacctgccatattttttggtccaagccttagtggtataaatcaagaaggctgtaaataagacttcagctttttggctggtgaagtttggttcc

(19) United States
(12) Patent Application Publication
(10) Pub. No.: US 2002/0081596 A1
(43) Pub. Date: Jun. 27, 2002

(54) Compositions and methods for the identification, assessment, prevention, and therapy of human cancers

(76) Inventors: James Lillie, Natick, MA (US); Jeffrey Brown, Arlington, MA (US); Andrew Bolt, Cambridge, MA (US); Christophe Van Huffel, Brussels (BE)

Correspondence Address: LAHIVE & COCKFIELD, 28 STATE STREET, BOSTON, MA, 02109, US

(21) Appl. No.: 09/816,292

(22) Filed: Mar. 22, 2001

Related U.S. Application Data
(60) Provisional application No. 60/192,100, filed on Mar. 24, 2000.
(60) Provisional application No. 60/197,064, filed on Apr. 13, 2000.

(51) Int. Cl. C12Q 1/68; C12Q 1/00; G01N 33/574

(52) U.S. Cl. 435/6; 435/4; 435/7.23

(57) ABSTRACT

The present invention is directed to the identification of markers that can be used to determine whether cancer cells are sensitive or resistant to a therapeutic agent. The present invention is also directed to the identification of therapeutic targets. The invention features a number of “sensitivity markers.” These are markers that are expressed in most or all cell lines that are sensitive to treatment with an agent and which are not expressed (or are expressed at a rather low level) in cells that are resistant to treatment with that agent. The invention also features a number of “resistance markers.” These are markers that are expressed in most or all cell lines that are resistant to treatment with an agent and which are not expressed (or are expressed at a rather low level) in cells that are sensitive to treatment with that agent.

### RELATED APPLICATIONS

The present application claims priority to U.S. provisional patent application Ser. No. 60/192,100, filed on Mar. 24, 2000, and U.S. provisional patent application Ser. No. 60/197,064, filed on Apr. 13, 2000, both of which are expressly incorporated by reference.

### BACKGROUND OF THE INVENTION

Cancers can be viewed as a breakdown in the communication between tumor cells and their environment, including their normal neighboring cells. Growth-stimulatory and growth-inhibitory signals are routinely exchanged between cells within a tissue. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, tumor cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature to supply nourishment and remove metabolic waste. Additionally, cells must acquire an ability to invade adjacent tissue. In many cases cells ultimately acquire the capacity to metastasize to distant sites.

It is apparent that the complex process of tumor development and growth must involve multiple gene products. It is therefore important to define the role of specific genes involved in tumor development and growth and identify those genes and gene products that can serve as targets for the diagnosis, prevention and treatment of cancers.

In the realm of cancer therapy it often happens that a therapeutic agent that is initially effective for a given patient becomes, over time, ineffective or less effective for that patient. The very same therapeutic agent may continue to be effective over a long period of time for a different patient. Further, a therapeutic agent that is effective, at least initially, for some patients can be completely ineffective or even harmful for other patients. Accordingly, it would be useful to identify genes and/or gene products that represent prognostic genes with respect to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures.

### SUMMARY OF THE INVENTION

The present invention is directed to the identification of markers that can be used to determine the sensitivity or resistance of cancer cells to a therapeutic agent. By examining the expression of one or more of the identified markers, whose expression correlates with sensitivity to a therapeutic agent or resistance to a therapeutic agent, in a sample of cancer cells, it is possible to determine whether a therapeutic agent or combination of agents will be most likely to reduce the growth rate of the cancer and can further be used in selecting appropriate treatment agents. The markers of the present invention whose expression correlates with sensitivity or with resistance to an agent are listed in Tables 1 and 2, respectively. Table 3 sets forth the markers of Tables 1 and 2 with their corresponding GenBank GI number.

By examining the expression of one or more of the identified markers in a sample of cancer cells, it is possible to determine which therapeutic agent or combination of agents will be most likely to reduce the growth rate of the cancer. By examining the expression of one or more of the identified markers in a sample of cancer cells, it is also possible to determine which therapeutic agent or combination of agents will be the least likely to reduce the growth rate of the cancer. By examining the expression of one or more of the identified markers, it is therefore possible to eliminate ineffective or inappropriate therapeutic agents. Moreover, by examining the expression of one or more of the identified markers in a sample of cancer cells taken from a patient during the course of therapeutic treatment, it is possible to determine whether the therapeutic treatment is continuing to be effective or whether the cancer has become resistant (refractory) to the therapeutic treatment. It is also possible to identify new anti-cancer agents by examining the expression of one or more markers when cancer cells or a cancer cell line is exposed to a potential anti-cancer agent. Importantly, these determinations can be made on a patient by patient basis or on an agent by agent (or combination of agents) basis. Thus, one can determine whether or not a particular therapeutic treatment is likely to benefit a particular patient or group/class of patients, or whether a particular treatment should be continued.

The present invention further provides previously unknown or unrecognized targets for the development of anti-cancer agents, such as chemotherapeutic compounds. The markers of the present invention can be used as targets in developing treatments (either single agent or multiple agent) for cancer, particularly for those cancers which display resistance to agents and exhibit expression of one or more of the markers identified herein, whose expression is correlated with resistance.

Other features and advantages of the invention will be apparent from the detailed description and from the claims. Although materials and methods similar or equivalent to those described herein can be used in the practice or testing of the invention, the preferred materials and methods are described below.

### DETAILED DESCRIPTION OF THE INVENTION

General Description

The present invention is based, in part, on the identification of markers that can be used to determine whether cancer cells are sensitive or resistant to a therapeutic agent. Based on these identifications, the present invention provides, without limitation: 1) methods for determining whether a therapeutic agent (or combination of agents) will or will not be effective in stopping or slowing tumor growth; 2) methods for monitoring the effectiveness of a therapeutic agent (or combination of agents) used for the treatment of cancer; 3) methods for identifying new therapeutic agents for the treatment of cancer; 4) methods for identifying combinations of therapeutic agents for use in treating cancer; and 5) methods for identifying specific therapeutic agents and combinations of therapeutic agents that are effective for the treatment of cancer in specific patients.

Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. The content of all GenBank and NUC database records cited throughout this application (including the Tables) are also hereby incorporated by reference. In the case of conflict, the present specification, including definitions, will control. In addition, the materials, methods, and examples are illustrative only and are not intended to be limiting.

The articles “a” and “an” are used herein to refer to one or to more than one (i.e. to at least one) of the grammatical object of the article. By way of example, “an element” means one element or more than one element.

A “marker” is a naturally-occurring polymer corresponding to at least one of the nucleic acids, or genetic loci, listed in Tables 1 or 2. For example, markers include, without limitation, sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (i.e. including proteins both before and after cleavage of normally cleaved regions such as transmembrane signal sequences). As used herein, “marker” may also include a cDNA made by reverse transcription of an RNA generated by transcription of genomic DNA (including spliced RNA).

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

The “normal” level of expression of a marker is the level of expression of the marker in cells of a patient not afflicted with cancer.

“Over-expression” and “under-expression” of a marker refer to expression of the marker of a patient at a greater or lesser level, respectively, than normal level of expression of the marker (e.g. at least two-fold greater or lesser level).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g. an RNA, a cDNA, or an analog of one of an RNA or cDNA) which is complementary to or homologous with all or a portion of a mature RNA made by transcription of a genomic DNA corresponding to a marker of the invention and normal post-transcriptional processing (e.g. splicing), if any, of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A marker is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature (e.g. encodes a natural protein).

Expression of a marker in a patient is “significantly” higher or lower than the normal level of expression of a marker if the level of expression of the marker is greater or less, respectively, than the normal level by an amount greater than the standard error of the assay employed to assess expression, and preferably at least twice, and more preferably three, four, five or ten times that amount. Alternately, expression of the marker in the patient can be considered “significantly” higher or lower than the normal level of expression if the level of expression is at least about two, and preferably at least about three, four, or five times, higher or lower, respectively, than the normal level of expression of the marker.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

A cancer cell is “sensitive” to a therapeutic agent if its rate of growth is inhibited as a result of contact with the therapeutic agent, compared to its growth in the absence of contact with the therapeutic agent. The quality of being sensitive to a therapeutic agent is a variable one, with different cancer cells exhibiting different levels of “sensitivity” to a given therapeutic agent, under different conditions. In one embodiment of the invention, cancer cells may be predisposed to sensitivity to an agent if one or more of the corresponding sensitivity markers (Table 1) are expressed.

A cancer cell is “resistant” to a therapeutic agent if its rate of growth is not inhibited, or inhibited to a very low degree, as a result of contact with the therapeutic agent when compared to its growth in the absence of contact with the therapeutic agent. The quality of being resistant to a therapeutic agent is a highly variable one, with different cancer cells exhibiting different levels of “resistance” to a given therapeutic agent, under different conditions. In another embodiments of the invention, cancer cells may be predisposed to resistance to an agent if one or more of the corresponding resistant markers (Table 2) are expressed.

A kit is any manufacture (e.g. a package or container) comprising at least one reagent, e.g. a probe, for specifically detecting a marker of the invention, the manufacture being promoted, distributed, or sold as a unit for performing the methods of the present invention. The reagents included in such a kit comprise probes/primers and/or antibodies for use in detecting sensitivity and resistance gene expression. In addition, the kits of the present invention may preferably contain instructions which describe a suitable detection assay. Such kits can be conveniently used, e.g., in clinical settings, to diagnose patients exhibiting symptoms of cancer.

### Specific Embodiments

I. Identification of Sensitivity and Resistance Genes

The present invention provides genes that are expressed in cancer cells that are sensitive or resistant to a given therapeutic agent and whose expression correlates with sensitivity to that therapeutic agent. The present invention also provides genes that are expressed in cancer cell lines that are resistant to a given therapeutic agent and whose expression correlates with sensitivity to that therapeutic agent. Accordingly, one or more of the sensitivity or resistance genes can be used as markers (or surrogate markers) to identify cancer cells that can be successfully treated by that agent. In addition, these markers can be used to identify cancers that have become or are at risk of becoming refractory to treatment with the agent.

II. Determining Sensitivity or Resistance to an Agent

The expression level of the identified sensitivity and resistance genes, or the proteins encoded by the identified sensitivity and resistance genes, may be used to: 1) determine if a cancer can be treated by an agent or combination of agents; 2) determine if a cancer is responding to treatment with an agent or combination of agents; 3) select an appropriate agent or combination of agents for treating a cancer; 4) monitor the effectiveness of an ongoing treatment; and 5) identify new cancer treatments (either single agent or combination of agents). In particular, the identified sensitivity and resistance genes may be utilized as markers (surrogate and/or direct) to determine appropriate therapy, to monitor clinical therapy and human trials of a drug being tested for efficacy, and to develop new agents and therapeutic combinations.

Accordingly, the present invention provides methods for determining whether an agent, e.g., a chemotherapeutic agent, can be used to reduce the growth rate of cancer cells comprising the steps of:

a) obtaining a sample of cancer cells;

b) determining whether the cancer cells express one or more markers identified in Tables 1 or 2 or both; and

c) identifying that an agent is or is not appropriate to treat the cancer based on the expression of the markers listed in Tables 1 or Table 2 or both.

In another embodiment, the invention provides a method for determining whether an agent can be used to reduce the growth of cancer cells, comprising the steps of:

a) obtaining a sample of cancer cells;

b) determining whether the cancer cells express one or more markers identified in Table 1; and

c) identifying that an agent is appropriate to treat the cancer when one or more markers listed in Table 1 are expressed by the cancer cells.

Alternatively, in step (c), an agent can be identified as not being appropriate to treat the cancer when one or more markers listed in Table 1 are not expressed by the cancer cells.

In another embodiment, the invention provides a method for determining whether an agent can be used to reduce the growth of cancer cells, comprising the steps of:

a) obtaining a sample of cancer cells;

b) determining whether the cancer cells express one or more markers identified in Table 2; and

c) identifying that an agent is appropriate to treat the cancer when one or more markers identified in Table 2 are not expressed by the cancer cells.

Alternatively, in step (c), an agent can be identified as not being appropriate to treat the cancer when one or more markers listed in Table 2 are expressed by the cancer cells.

In another embodiment, the invention provides a method for determining whether an agent can be used to reduce the growth of cancer cells, comprising the steps of:

a) obtaining a sample of cancer cells;

b) exposing some of the cancer cells to one or more test agents;

c) determining the level of expression in of one or more markers listed in Table 1 both in cancer cells exposed to the agent and in cancer cells that have not been exposed to the agent; and

d) identifying that an agent is appropriate to treat the cancer when the expression of the markers listed in Table 1 is increased in the presence of the agent.

Alternatively, in step (d), an agent can be identified as not being appropriate to treat the cancer when the expression of the markers listed in Table 1 is decreased in the presence of the agent.

In another embodiment, the invention provides a method for determining whether an agent can be used to reduce the growth of cancer cells, comprising the steps of:

a) obtaining a sample of cancer cells;

b) exposing some of the cancer cells to one or more test agents;

c) determining the level of expression in of one or more markers listed in Table 2 both in cancer cells exposed to the agent and in cancer cells that have not been exposed to the agent; and

d) identifying that an agent is not appropriate to treat the cancer when the expression of the markers listed in Table 2 is increased in the presence of the agent.

Alternatively, in step (d), an agent can be identified as being appropriate to treat the cancer when the expression of the markers listed in Table 2 is decreased in the presence of the agent.

In another embodiment, the invention provides a method for determining whether treatment with an anti-cancer agent should be continued in a cancer patient, comprising the steps of:

a) obtaining two or more samples of cancer cells from a patient at different times during the course of anti-cancer agent treatment;

b) determining the level of expression in the cancer cells of one or more genes which correspond to markers listed in Table 1 in the two or more samples; and

c) continuing the treatment when the expression level of the markers listed in Table 1 does not decrease during the course of treatment.

Alternatively, in step (c), the treatment is discontinued when the expression level of the markers listed in Table 1 is decreased during the course of treatment.

In another embodiment, the invention provides a method for determining whether treatment with an anti-cancer agent should be continued in a cancer patient, comprising the steps of:

a) obtaining two or more samples of cancer cells from a patient at different times during the course of anti-cancer agent treatment;

b) determining the level of expression in the cancer cells of one or more markers listed in Table 2 in the two or more samples; and

c) continuing the treatment when the expression level of one or more markers listed in Table 2 is not increased during the course of treatment.

Alternatively, in step (c), the treatment is discontinued when the expression level of one or more markers listed in Table 2 is increased during the course of treatment.

In another embodiment of the invention, the agent used in methods of the invention is a taxane compound. In another embodiment of the invention, the expression of genes which correspond to markers listed in Table 1 or Table 2 or both is detected by measuring mRNA which corresponds to the gene. In yet another embodiment of the invention, the expression of genes which correspond to markers listed in Table 1 or Table 2 or both is detected by measuring protein which corresponds to the gene. In a further another embodiment of the invention, the cancer cells or cancer cell lines used in the methods of the invention are obtained from a patient.

In another embodiment, the invention provides a method of treating a patient for cancer by administering to the patient a compound which has been identified as being effective against cancer by methods of the invention described herein.

As used herein, an agent is said to reduce the rate of growth of cancer cells when the agent can reduce at least 50%, preferably at least 75%, most preferably at least 95% of the growth of the cancer cells.

Such inhibition can further include a reduction in survivability and an increase in the rate of death of the cancer cells. The amount of agent used for this determination will vary based on the agent selected. Typically, the amount will be a predefined therapeutic amount.

As used herein, the term “agent” is defined broadly as anything that cancer cells may be exposed to in a therapeutic protocol. In the context of the present invention, such agents include, but are not limited to, chemotherapeutic agents, such as anti-metabolic agents, e.g., Ara AC, 5-FU and methotrexate, antimitotic agents, e.g., TAXOL, inblastine and vincristine, alkylating agents, e.g., melphanlan, BCNU and nitrogen mustard, Topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light. In a preferred embodiment, the agent is a taxane compound (e.g., TAXOL).

Further to the above, the language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table A.

TABLE A

| | | NONPROPRIETARY |
|---|---|---|
| | | NAMES |
| CLASS | TYPE OF AGENT | (OTHER NAMES) |
| Alkylating | Nitrogen Mustards | Mechlorethamine (HN₂) |
| | | Cyclophosphaniide |
| | | Ifosfamide |
| | | Melphalan (L-sarcolysin) |
| | | Chlorambucil |
| | Ethylenimines | Hexamethylmelamine |
| | And Methylmelamines | Thiotepa |
| | Alkyl Sulfonates | Busulfan |
| Alkylating | Nitrosoureas | Carmustine (BCNU) |
| | | Lomustine (CCNU) |
| | | Semustine (methyl-CCNU) |
| | | Streptozocin |
| | | (streptozotocin) |
| | Triazenes | Decarbazine (DTIC; |
| | | dimethyltriazenoimi- |
| | | dazolecarboxamide) |
| | Alkylator | cis-diamminedichloroplatinum |
| | | II (CDDP) |
| Antimeta- | Folic Acid | Methotrexate |
| bolites | Analogs | (amethopterin) |
| | Pyrimidine | Fluorouracil |
| | Analogs | (′5-fluorouracil 5-FU) |
| | Floxuridine (fluorode-oxyuridine; | |
| | | FUdR) |
| | | Cytarabine (cytosine |
| | | arabinoside) |
| | Purine Analogs | Mercaptopuine |
| | and Related | (6-mercaptopurine; |
| | Inhibitors | 6-MP) |
| | | Thioguanine |
| | | (6-thioguanine; TG) |
| | | Pentostatin (2′ - deoxycoformycin) |
| Natural | Vinca Alkaloids | Vinblastin (VLB) |
| Products | | Vincristine |
| | Topoisomerase | Etoposide |
| | Inhibitors | Teniposide |
| | | Camptothecin |
| | | Topotecan |
| | | 9-amino-campotothecin CPT-11 |
| | Antibiotics | Dactinomycin |
| | | (actinomycin D) |
| | | Adriamycin |
| | | Daunorubicin |
| | | (daunomycin; |
| | | rubindomycin) |
| | | Doxorubicin |
| | | Bleomycin |
| | | Plicamycin |
| | | (mithramycin) |
| | | Mitomycin (mitomycin C) |
| | | TAXOL |
| | | Taxotere |
| | Enzymes | L-Asparaginase |
| | Biological | Interfon alfa |
| | Response | interleukin 2 |
| | Modifiers | |
| Miscella- | Platinum | cis-diamminedichloroplatinum |
| neous | Coordination | II (CDDP) |
| Agents | Complexes | Carboplatin |
| | Anthracendione | Mitoxantrone |
| | Substituted Urea | Hydroxyurea |
| | Methyl Hydraxzine | Procarbazine |
| | Derivative | (N-methylhydrazine, |
| | | (MIH) |
| | Adrenocortical | Mitotane |
| | Suppressant | (o,p′-DDD) |
| | | Aminoglutehimide |
| Hormones | Adrenocorticosteroids | Prednisone |
| and | Progestins | Hydroxyprogesterone |
| Antagonists | | caproate |
| | | Medroxyprogesterone |
| | | acetate |
| | | Megestrol acetate |
| | Estrogens | Diethylstilbestrol |
| | | Ethinyl estradiol |
| | Antiestrogen | Tamoxifen |
| | Androgens | Testosterone propionate |
| | | Fluoxymesterone |
| | Antiandrogen | Flutamide |
| | Gonadotropin-releasing | Leuprolide |
| | Hormone analog | |

The agents tested in the present methods can be a single agent or a combination of agents. For example, the present methods can be used to determine whether a single chemotherapeutic agent, such as methotrexate, can be used to treat a cancer or whether a combination of two or more agents can be used. Preferred combinations will include agents that have different mechanisms of action, e.g., the use of an anti-mitotic agent in combination with an alkylating agent.

As used herein, cancer cells refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningoma, medulloblastoma, schwannoma or epidymoma.

The source of the cancer cells used in the present method will be based on how the method of the present invention is being used. For example, if the method is being used to determine whether a patient's cancer can be treated with an agent, or a combination of agents, then the preferred source of cancer cells will be cancer cells obtained from a cancer biopsy from the patient. Alternatively, a cancer cell line similar to the type of cancer being treated can be assayed. For example if breast cancer is being treated, then a breast cancer cell line can be used. If the method is being used to monitor the effectiveness of a therapeutic protocol, then a tissue sample from the patient being treated is the preferred source. If the method is being used to identify new therapeutic agents or combinations, any cancer cells, e.g., cells of a cancer cell line, can be used.

A skilled artisan can readily select and obtain the appropriate cancer cells that are used in the present method. For cancer cell lines, sources such as The National Cancer Institute, for the NCI-60 cells used in the examples, are preferred. For cancer cells obtained from a patient, standard biopsy methods, such as a needle biopsy, can be employed.

In the methods of the present invention, the level or amount of expression of one or more genes selected from the group consisting of the genes identified in Table 1 and 2 is determined. As used herein, the level or amount of expression refers to the absolute level of expression of an mRNA encoded by the gene or the absolute level of expression of the protein encoded by the gene (i.e., whether or not expression is or is not occurring in the cancer cells).

Generally, it is preferable to determine the expression of two or more of the identified sensitivity or resistance genes, more preferably, three or more of the identified sensitivity or resistance genes, most preferably all of the identified sensitivity and/or resistance genes. Thus, it is preferable to assess the expression of a panel of sensitivity and resistance genes.

As an alternative to making determinations based on the absolute expression level of selected genes, determinations may be based on the normalized expression levels. Expression levels are normalized by correcting the absolute expression level of a sensitivity or resistance gene by comparing its expression to the expression of a gene that is not a sensitivity or resistance gene, e.g., a housekeeping genes that is constitutively expressed. Suitable genes for normalization include housekeeping genes such as the actin gene. This normalization allows one to compare the expression level in one sample, e.g., a patient sample, to another sample, e.g., a non-cancer sample, or between samples from different sources.

Alternatively, the expression level can be provided as a relative expression level. To determine a relative expression level of a gene, the level of expression of the gene is determined for 10 or more samples, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of sensitivity or resistance.

Preferably, the samples used will be from similar tumors or from non-cancerous cells of the same tissue origin as the tumor in question. The choice of the cell source is dependent on the use of the relative expression level data. For example, using tumors of similar types for obtaining a mean expression score allows for the identification of extreme cases of sensitivity or resistance. Using expression found in normal tissues as a mean expression score aids in validating whether the sensitivity/resistance gene assayed is tumor specific (versus normal cells). Such a later use is particularly important in identifying whether a sensitivity or resistance gene can serve as a target gene. In addition, as more data is accumulated, the mean expression value can be revised, providing improved relative expression values based on accumulated data.

III. Isolated Nucleic Acid Molecules

One aspect of the invention pertains to isolated nucleic acid molecules that correspond to a marker of the invention, including nucleic acids which encode a polypeptide corresponding to a marker of the invention or a portion of such a polypeptide. Isolated nucleic acids of the invention also include nucleic acid molecules sufficient for use as hybridization probes to identify nucleic acid molecules that correspond to a marker of the invention, including nucleic acids which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid encoding a protein corresponding to a marker listed in Tables 1 and/or 2, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a protein which corresponds to a marker of the invention, and thus encode the same protein.

In addition to the nucleotide sequences described in the GenBank and NUC database records described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid corresponding to a marker of the invention or to a nucleic acid encoding a protein corresponding to a marker of the invention. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6×sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August:1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into an ovary-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al, 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

IV. Isolated Proteins and Antibodies

One aspect of the invention pertains to isolated proteins which correspond to individual markers of the invention, and biologically active portions thereof, as well as polypeptide fragments suitable for use as immunogens to raise antibodies directed against a polypeptide corresponding to a marker of the invention. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the marker (e.g., the amino acid sequence listed in the GenBank and NUC database records described herein), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence listed in the one of the GenBank and NUC database records described herein. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total# of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al, supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual markers of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies of the invention may be used as therapeutic agents in treating cancers. In a preferred embodiment, completely human antibodies of the invention are used for therapeutic treatment of human cancer patients, particularly those having an cancer. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g. in an ovary-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2 ×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a polypeptide of the present invention, the method comprising immunizing a mammal with a polypeptide. The polypeptide used as an immnungen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C.

After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

V. Recombinant Expression Vectors and Host Cells

Another aspect of the invention pertains to vectors, preferably expression vectors, containing a nucleic acid encoding a polypeptide corresponding to a marker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide corresponding to a marker of the invention have been introduced. Such host cells can then be used to create non-human transgenic animals in which exogenous sequences encoding a marker protein of the invention have been introduced into their genome or homologous recombinant animals in which endogenous gene(s) encoding a polypeptide corresponding to a marker of the invention sequences have been altered. Such animals are useful for studying the function and/or activity of the polypeptide corresponding to the marker and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid encoding a polypeptide corresponding to a marker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a marker of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells: A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

VI. Pharmaceutical Compositions

The nucleic acid molecules, polypeptides, and antibodies (also referred to herein as “active compounds”) corresponding to a marker of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker or biologically active portion thereof. In another embodiment, the invention provides assays for screening candidate or test compounds which bind to a marker or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker or a biologically active portion thereof. In all likelihood, the marker can, in vivo, interact with one or more molecules, such as but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of the marker to identify its natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al , 1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker or downstream elements of a marker-mediated signaling pathway. Alternatively, such marker binding partners may also be found to be inhibitors of the marker.

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is an cancer marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner. Conversely, the formation of more complex in the presence of compound than in the control reaction indicates that the compound may enhance interaction of the marker and its binding partner.

The assay for compounds that interfere with the interaction of the marker with its binding partner may be conducted in a heterogeneous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J. Mol. Recognit. 11I:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein. In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an marker modulating agent, an antisense marker nucleic acid molecule, an marker-specific antibody, or an marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the epithelium). A method for lipidation of antibodies is described by Cruikshank et al. (1997) J. Acquired Immune Deficiency Syndromes and Human Retrovirology 14:193.

The nucleic acid molecules corresponding to a marker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

VII. Monitoring the Effectiveness of an Anti-Cancer Agent

As discussed above, the identified sensitivity and resistance genes can also be used as markers to assess whether a tumor has become refractory to an ongoing treatment (e.g., a chemotherapeutic treatment). When a tumor is no longer responding to a treatment the expression profile of the tumor cells will change: the level of expression of one or more of the sensitivity genes will be reduced and the level of expression of one or more of the resistance genes will increase.

In such a use, the invention provides methods for determining whether an anti-cancer treatment should be continued in a cancer patient, comprising the steps of:

a) obtaining two or more samples of cancer cells from a patient undergoing anti-cancer therapy;

b) determining the level of expression of one or more genes selected from the group consisting of the sensitivity genes (Table 1) and the resistance genes (Table 2) in the sample exposed to the agent and in a sample of cancer cells that is not exposed to the agent; and

c) discontinuing or altering treatment when the expression of one or more sensitivity genes decreases or when the expression of one or more resistance genes increases.

As used here, a patient refers to any subject undergoing treatment for cancer. The preferred subject will be a human patient undergoing chemotherapy treatment.

This embodiment of the present invention relies on comparing two or more samples obtained from a patient undergoing anti-cancer treatment. In general, it is preferable to obtain a first sample from the patient prior to beginning therapy and one or more samples during treatment. In such a use, a baseline of expression prior to therapy is determined and then changes in the baseline state of expression is monitored during the course of therapy. Alternatively, two or more successive samples obtained during treatment can be used without the need of a pretreatment baseline sample. In such a use, the first sample obtained from the subject is used as a baseline for determining whether the expression of a particular gene is increasing or decreasing.

In general, when monitoring the effectiveness of a therapeutic treatment, two or more samples from the patient are examined. Preferably, three or more successively obtained samples are used, including at least one pretreatment sample.

VIII. Detection Assays

An exemplary method for detecting the presence or absence of a polypeptide or nucleic acid corresponding to a marker of the invention in a biological sample involves obtaining a biological sample (e.g. an ovary-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11 (1-6):141-8; Hage, D. S., and Tweed, S. A. J Chromatogr B Biomed Sci Appl 1997 October 10;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cancer or from non-cancer cells of tissue. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data. Expression data from cells provides a means for grading the severity of the cancer state.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cells express a marker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g. an ovary-associated body fluid such as a urine sample). Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

IX. Electronic Apparatus Readable Media and Arrays

Electronic apparatus readable media comprising a marker of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the markers of the present invention.

A variety of software programs and formats can be used to store the marker information of the present invention on the electronic apparatus readable medium. For example, the nucleic acid sequence corresponding to the markers can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the markers of the present invention.

By providing the markers of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The invention also includes an array comprising a marker of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of breastcancer, progression of cancer, and processes, such a cellular transformation associated with cancer.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

### SPECIFIC EXAMPLES

At least some of the examples set forth below relate to sensitivity or resistance to TAXOL. TAXOL is a chemical compound within a family of taxane compounds which are art-recognized as being a family of related compounds. The language “taxane compound” is intended to include TAXOL, compounds which are structurally similar to TAXOL and/or analogs of TAXOL. The language “taxane compound” can also include “mimics”. “Mimics” is intended to include compounds which may not be structurally similar to TAXOL but mimic the therapeutic activity of TAXOL or structurally similar taxane compounds in vivo. The taxane compounds of this invention are those compounds which are useful for inhibiting tumor growth in subjects (patients). The term taxane compound also is intended to include pharmaceutically acceptable salts of the compounds. Taxane compounds have previously been described in U.S. Pat. Nos. 5,641,803, 5,665,671, 5,380,751, 5,728,687, 5,415,869, 5,407,683, 5,399,363, 5,424,073, 5,157,049, 5,773,464, 5,821,263, 5,840,929, 4,814,470, 5,438,072, 5,403,858, 4,960,790, 5,433,364, 4,942,184, 5,362,831, 5,705,503, and 5,278,324, all of which are expressly incorporated by reference.

The structure of TAXOL, shown below, offers many groups capable of being synthetically functionalized to alter the physical or pharmaceutical properties of TAXOL.

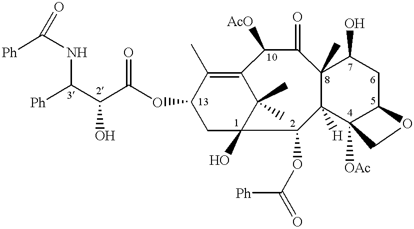

For example, a well known semi-synthetic analog of TAXOL, named Taxotere (docetaxel), has also been found to have good anti-tumor activity in animal models. Taxotere has t-butoxy amide at the 3′ position and a hydroxyl group at the C10 position (U.S. Pat. No. 5,840,929).

Other examples of TAXOL derivatives include those mentioned in U.S. Pat. No. 5,840,929 which are directed to derivatives of TAXOL having the formula:

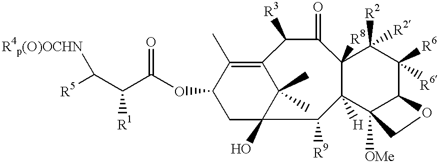

wherein R¹ is hydroxy, —OC(O)R^{x}, or —OC(O)OR^{x}; R² is hydrogen, hydroxy, —OC(O)R^{x}, or —OC(O)OR^{x}; R^{2′} is hydrogen, hydroxy, or fluoro; R^{6′} is hydrogen or hydroxy or R^{2′} and R^{6′} can together form an oxirane ring; R³ is hydrogen, C₁₋₆ alkyloxy, hydroxy, —OC(O)R^{x}, —OC(O)OR^{x}, —OCONR⁷R¹¹; R⁸ is methyl or R⁸ and R² together can form a cyclopropane ring; R⁶ is hydrogen or R⁶ and R² can together form a bond; R⁹ is hydroxy or —OC(O)R^{x}; R⁷ and R¹¹ are independently C₁₋₆ alkyl, hydrogen, aryl, or substituted aryl; R⁴ and R⁵ are independently C₁₋₆ alkyl, C₂₋₆ alkenyl, C₂₋₆ alkynyl, or —Z—R¹⁰; Z is a direct bond, C₁₋₆ alkyl, or C₂₋₆ alkenyl; R¹⁰ is aryl, substituted aryl, C₃₋₆ cycloalkyl, C₂₋₆ alkenyl, C₁₋₆ alkyl, all can be optionally substituted with one to six same or different halogen atoms or hydroxy; R^{x} is a radical of the formula:

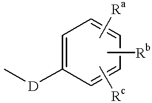

wherein D is a bond or C₁₋₆ alkyl; and R^{a}, R^{b} and R^{c} are independently hydrogen, amino, C₁₋₆ alkyl or C₁₋₆ alkoxy.

Further examples of R^{x} include methyl, hydroxymethyl, ethyl, n-propyl, isopropyl, n-butyl, isobutyl, chloromethyl, 2,2,2-trichloroethyl, cyclopropyl, cyclobutyl, cyclopentyl, cyclohexyl, ethenyl, 2-propenyl, phenyl, benzyl, bromophenyl, 4-aminophenyl, 4-methylaminophenyl, 4-methylphenyl, 4-methoxyphenyl and the like. Examples of R⁴ and R⁵ include 2-propenyl, isobutenyl, 3-furanyl (3-furyl), 3-thienyl, phenyl, naphthyl, 4-hydroxyphenyl, 4-methoxyphenyl, 4-fluorophenyl, 4-trifluoromethylphenyl, methyl, ethyl, n-propyl, isopropyl, n-butyl, isobutyl, t-butyl, ethenyl, 2-propenyl, 2-propynyl, benzyl, phenethyl, phenylethenyl, 3,4-dimethoxyphenyl, 2-furanyl (2-furyl), 2-thienyl, 2-(2-furanyl)ethenyl, 2-methylpropyl, cyclopropyl, cyclobutyl, cyclopentyl, cyclohexyl, cyclohexylmethyl, cyclohexylethyl and the like.

TAXOL derivatives can be readily made by following the well established paclitaxel chemistry. For example, C2, C6, C7, C10, and/or C8 position can be derivatized by essentially following the published procedure, into a compound in which R³, R⁸, R², R^{2′}, R⁹, R^{6′} and R⁶ have the meanings defined earlier. Subsequently, C4-acetyloxy group can be converted to the methoxy group by a sequence of steps. For example, for converting C2-benzoyloxy to other groups see, S. H. Chen et al, Bioorganic and Medicinal Chemistry Letters, Vol. 4, No. 3, pp 479-482 (1994); for modifying C10-acetyloxy see, J. Kant et al, Tetrahedron Letters, Vol. 35, No. 31, pp 5543-5546 (1994) and U.S. Pat. No. 5,294,637 issued Mar. 15, 1994; for making C10 and/or C7 unsubstituted (deoxy) derivatives see, European Patent Application 590 267A2 published Apr. 6, 1994 and PCT application WO 93/06093 published Apr. 1, 1993; for making 7β,8β-methano, 6,7-α,α-dihydroxy and 6,7-olefinic groups see, R. A. Johnson, Tetrahedron Letters, Vol. 35, No 43, pp 7893-7896 (1994), U.S. Pat. No. 5,254,580, issued Oct. 19, 1993, and European Patent Application 600 517A1 published Jun. 8, 1994; for making C7/C6 oxirane see, U.S. Pat. No. 5,395,850 issued Mar. 7, 1995; for making C7-epi-fluoro see, G. Roth et al, Tetrahedron Letters, Vol 36, pp 1609-1612 (1993); for forming C7 esters and carbonates see, U.S. Pat. No. 5,272,171 issued Dec. 21, 1993 and S. H. Chen et al., Tetrahedron, 49, No. 14, pp 2805-2828 (1993).

In U.S. Pat. No. 5,773,464, TAXOL derivatives containing epoxides at the C₁₀ position are disclosed as antitumor agents. Other C-10 taxane analogs have also appeared in the literature. Taxanes with alkyl substituents at C-10 have been reported in a published PCT patent application WO 9533740. The synthesis of C-10 epi hydroxy or acyloxy compounds is disclosed in PCT application WO 96/03394. Additional C-10 analogs have been reported in Tetrahedron Letters 1995, 36(12), 1985-1988; J. Org. Chem. 1994, 59, 4015-4018 and references therein; K. V. Rao et. al. Journal of Medicinal Chemistry 1995, 38 (17), 3411-3414; J. Kant et. al. Tetrahedron Lett. 1994, 35(31), 5543-5546; WO 9533736; WO 93/02067; U.S. Pat. No. 5,248,796; WO 9415929; and WO 94/15599.

Other relevant TAXOL derivatives include the sulfenamide taxane derivatives described in U.S. Pat. No. 5,821,263. These compounds are charachterized by the C3′ nitrogen bearing one or two sulfur substiuents. These compounds have been useful in the treatment of cancers such as ovarian, breast, lung, gastic, colon, head, neck, melanoma, and leukemia.

U.S. Pat. No. 4,814,470 discusses TAXOL derivatives with hydroxyl or acetyl group at the C10 position and hydroxy or t-butylcarbonyl at C2′ and C3′ positions.

U.S. Pat. No. 5,438,072 discusses TAXOL derivatives with hydroxyl or acetate groups at the C10 position and a C2′ substitutuent of either t-butylcarbonyl or benzoylamino.

U.S. Pat. No. 4,960,790 discusses derivatives of TAXOL which have, at the C2′ and/or C7 position a hydrogen, or the residue of an amino acid selected from the group consisting of alanine, leucine, isoleucine, saline, phenylalanine, proline, lysine, and arginine, or a group of the formula:

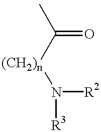

wherein n is an integer of 1 to 3 and R² and R³ are each hydrogen on an alkyl radical having one to three carbon atoms or wherein R² and R³ together with the nitrogen atom to which they are attached form a saturated heterocyclic ring having four to five carbon atoms, with the proviso that at least one of the substituents are not hydrogen.

Other similar water soluble TAXOL derivatives are discussed in U.S. Pat. Nos. 4,942,184, 5,433,364, and in 5,278,324.

Many TAXOL derivatives may also include protecting groups such as, for example, hydroxy protecting groups. “Hydroxy protecting groups” include, but are not limited to, ethers such as methyl, t-butyl, benzyl, p-methoxybenzyl, p-nitrobenzyl, allyl, trityl, methoxymethyl, methoxyethoxymethyl, ethoxyethyl, tetrahydropyranyl, tetrahydrothiopyranyl, dialkylsilylethers, such as dimethylsilyl ether, and trialkylsilyl ethers such as trimethylsilyl ether, triethylsilyl ether, and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates such as methyl, ethyl, 2,2,2-trichloroethyl, allyl, benzyl, and p-nitrophenyl. Additional examples of hydroxy protecting groups may be found in standard reference works such as Greene and Wuts, Protective Groups in Organic Synthesis, 2d Ed., 1991, John Wiley & Sons, and McOmie; and Protective Groups in Organic Chemistry, 1975, Plenum Press. Methods for introducing and removing protecting groups are also found in such textbooks.

A. Generation of Subtracted Libraries

Subtracted libraries are generated using a PCR based method that allows the isolation of clones expressed at higher levels in one population of mRNA (tester) compared to another population (driver). Both tester and driver mRNA populations are converted into cDNA by reverse transcription, and then PCR amplified using the SMART PCR kit from Clontech. Tester and driver cDNAs are then hybridized using the PCR-Select cDNA subtraction kit from Clontech. This technique results in both subtraction and normalization, which is an equalization of copy number of low-abundance and high-abundance sequences. After generation of the subtractive libraries, a group of 96 or more clones from each library is tested to confirm differential expression by reverse Southern hybridization.

RNA was generated and pooled from two groups of cancer cell lines shown in Tables B and C. One group of nine cell lines was determined to be sensitive to TAXOL (Table C), the other group of nine cell lines was determined to be resistant to TAXOL (Table B). Sensitivity to TAXOL was based of known GI₅₀ values for these cells, which for this study was defined as the concentration of TAXOL required to inhibit growth of the cell line by 50%. More precisely, the quantity used in the calculation is the potency measure −log{GI₅₀}. Pooled RNA from TAXOL sensitive cancer cell lines was used as tester against driver RNA pooled from TAXOL resistant cancer cell lines. The results of this subtractive library are shown in Table 1. Pooled RNA from TAXOL resistant cancer cell lines was used as tester against driver RNA pooled from TAXOL sensitive cancer cell lines. The results of this subtractive library are shown in Table 2.

Tables 1 and 2 show the accession number (“Accession #”) of the markers of the present invention. The accession number is the identification number assigned to the marker in the relevant database (see, e.g. http://www.ncbi.nlm.nih.gov/genbank/guery form.html). Table 3 shows the accession number (“Acc. No.”) of the markers of the present invention with the corresponding GenBank GI number (“GI No.”) The GenBank GI number is the identification number assigned the marker in the GenBank database (see supra). One skilled in the art can thus obtain from the Tables of the present invention both the GenBank accession number as well as the GenBank GI number for a marker of the present invention, thereby identifying the nucleotide and/or polypeptide sequence of that marker.

TABLE B

| | TAXOL | |
|---|---|---|
| | Resistant | Log Gl 50 for |
| Tissue of Origin | Cell Line | TAXOL |
| Non-small cell lung | EKVX | −6.6 |
| carcinoma | | |
| Non-small cell lung | HOP-92 | −7.2 |
| carcinoma | | |
| Colon | HCT-15 | −6.7 |
| Melanoma | MALME-3M | −6.8 |
| Melanoma | SK-MEL-28 | −7.1 |
| Ovarian | OVCAR-4 | −6.3 |
| Renal | ACHN | −5.8 |
| Breast | MCF- | −5.5 |
| | 7/AdrRes | |
| Breast | T-47D | −6.9 |
| | | −6.5 |
| | | (Mean) |

TABLE C

| | TAXOL | |
|---|---|---|
| | Sensitive | Log GI 50 for |
| Tissue of Origin | Cell Line | TAXOL |
| Non-small cell lung | NCl-H460 | −8.5 |
| carcinoma | | |
| Non-small cell lung | NCl-H522 | −8.5 |
| carcinoma | | |
| Colon | HT-29 | −8.6 |
| Melanoma | SK-MEL-2 | −8.3 |
| Melanoma | SK-MEL-5 | −8.4 |
| Ovarian | OVCAR-3 | −8.5 |
| Renal | SN12C | −8.5 |
| Breast | MCF-7 | −8.5 |
| Breast | MDA-MB- | −8.6 |
| | 435 | |
| | | −8.5 |
| | | (Mean) |

B. Sensitivity Assays and Identification of Therapeutic and Drug Screening Targets

A sample of cancerous cells with unknown sensitivity to a given drug is obtained from a patient. An expression level is measured in the sample for a gene corresponding to one of the markers identified in either Table 1 and/or in Table 2. If the gene is expressed, and the marker of the invention to which the gene corresponds is listed among the markers of Table 1, then the drug will be effective against the cancer. Accordingly, if the gene is not expressed, and the marker of the invention to which the gene corresponds is listed among in the markers of Table 1, then the drug will not be effective against the cancer. If the gene is expressed, and the marker of the invention to which the gene corresponds is listed among the markers of Table 2, then the drug will not be effective against the cancer. Accordingly, if the gene is not expressed, and the marker of the invention to which the gene corresponds is listed among the markers of Table 2, then the drug will be effective against the cancer.

Thus, by examining the expression of one or more of the identified markers in a sample of cancer cells, it is possible to determine which therapeutic agent(s), or combination of agents, to use as the appropriate treatment agents.

By examining the expression of one or more of the identified markers in a sample of cancer cells taken from a patient during the course of therapeutic treatment, it is also possible to determine whether the therapeutic agent is continuing to work or whether the cancer has become resistant (refractory) to the treatment protocol. For example, a cancer patient receiving a treatment of paclitaxel would have cancer cells removed and monitored for the expression of a marker. If the expression level of a marker remains substantially the same, the treatment with paclitaxel would continue. However, a significant change in marker expression would suggest that the cancer may have become resistant to paclitaxel and another chemotherapy protocol should be initiated to treat the patient.

Importantly, these determinations can be made on a patient by patient basis or on an agent by agent (or combinations of agents). Thus, one can determine whether or not a particular therapeutic treatment is likely to benefit a particular patient or group/class of patients, or whether a particular treatment should be continued.

The identified markers further provide previously unknown or unrecognized targets for the development of anti-cancer agents, such as chemotherapeutic compounds, and can be used as targets in developing single agent treatment as well as combinations of agents for the treatment of cancer.

Other Embodiments

The present invention is not to be limited in scope by the specific embodiments described that are intended as single illustrations of individual aspects of the invention and functionally equivalent methods and components are within the scope of the invention, in addition to those shown and described herein will become apparent to those skilled in the art from the foregoing description and accompanying drawings. Such modifications are intended to fall within the scope of the appended claims.

All references cited herein, including journal articles, patents, and databases are expressly incorporated by reference.

TABLE 1

| Sequence ID | Accession # |
|---|---|
| Sequence 1 | AA001696 |
| Sequence 2 | AA002128 |
| Sequence 3 | AA009923 |
| Sequence 4 | AA010575 |
| Sequence 5 | AA010689 |
| Sequence 6 | AA010893 |
| Sequence 7 | AA011002 |
| Sequence 8 | AA022748 |
| Sequence 9 | AA022943 |
| Sequence 10 | AA025349 |
| Sequence 11 | AA028882 |
| Sequence 12 | AA034237 |
| Sequence 13 | AA036750 |
| Sequence 14 | AA037181 |
| Sequence 15 | AA040929 |
| Sequence 16 | AA043103 |
| Sequence 17 | AA043137 |
| Sequence 18 | AA045176 |
| Sequence 19 | AA046473 |
| Sequence 20 | AA046810 |
| Sequence 21 | AA046888 |
| Sequence 22 | AA047054 |
| Sequence 23 | AA054771 |
| Sequence 24 | AA056334 |
| Sequence 25 | AA058936 |
| Sequence 26 | AA069078 |
| Sequence 27 | AA069560 |
| Sequence 28 | AA069850 |
| Sequence 29 | AA071084 |
| Sequence 30 | AA074035 |
| Sequence 31 | AA074291 |
| Sequence 32 | AA074845 |
| Sequence 33 | AA075527 |
| Sequence 34 | AA081348 |
| Sequence 35 | AA082884 |
| Sequence 36 | AA083270 |
| Sequence 37 | AA083410 |
| Sequence 38 | AA085444 |
| Sequence 39 | AA085511 |
| Sequence 40 | AA088344 |
| Sequence 41 | AA088758 |
| Sequence 42 | AA095772 |
| Sequence 43 | AA099904 |
| Sequence 44 | AAI00707 |
| Sequence 45 | AAI01783 |
| Sequence 46 | AAI02138 |
| Sequence 47 | AAI02721 |
| Sequence 48 | AAI02853 |
| Sequence 49 | AAI13420 |
| Sequence 50 | AAI15218 |
| Sequence 51 | AAI15838 |
| Sequence 52 | AAI21574 |
| Sequence 53 | AAI25927 |
| Sequence 54 | AAI27186 |
| Sequence 55 | AAI28091 |
| Sequence 56 | AAI28878 |
| Sequence 57 | AAI28965 |
| Sequence 58 | AAI30823 |
| Sequence 59 | AAI31227 |
| Sequence 60 | AAI32844 |
| Sequence 61 | AAI36789 |
| Sequence 62 | AAI42909 |
| Sequence 63 | AAI43438 |
| Sequence 64 | AAI43746 |
| Sequence 65 | AAI47080 |
| Sequence 66 | AAI49963 |
| Sequence 67 | AAI56443 |
| Sequence 68 | AAI56615 |
| Sequence 69 | AAI56616 |
| Sequence 70 | AAI57300 |
| Sequence 71 | AAI60517 |
| Sequence 72 | AAI61269 |
| Sequence 73 | AAI66632 |
| Sequence 74 | AAI66675 |
| Sequence 75 | AAI67700 |
| Sequence 76 | AAI67814 |
| Sequence 77 | AAI73279 |
| Sequence 78 | AAI74034 |
| Sequence 79 | AAI76813 |
| Sequence 80 | AAI79439 |
| Sequence 81 | AAI81153 |
| Sequence 82 | AAI81811 |
| Sequence 83 | AAI81858 |
| Sequence 84 | AAI87817 |
| Sequence 85 | AAI88680 |
| Sequence 86 | AAI88832 |
| Sequence 87 | AAI91341 |
| Sequence 88 | AAI95178 |
| Sequence 89 | AAI96515 |
| Sequence 90 | AAI99684 |
| Sequence 91 | AA203284 |
| Sequence 92 | AA205412 |
| Sequence 93 | AA211509 |
| Sequence 94 | AA211584 |
| Sequence 95 | AA213580 |
| Sequence 96 | AA215584 |
| Sequence 97 | AA216094 |
| Sequence 98 | AA223381 |
| Sequence 99 | AA224124 |
| Sequence 100 | AA224163 |
| Sequence 101 | AA225289 |
| Sequence 102 | AA226279 |
| Sequence 103 | AA235063 |
| Sequence 104 | AA235365 |
| Sequence 105 | AA251312 |
| Sequence 106 | AA256442 |
| Sequence 107 | AA262249 |
| Sequence 108 | AA278456 |
| Sequence 109 | AA279497 |
| Sequence 110 | AA280091 |
| Sequence 111 | AA281007 |
| Sequence 112 | AA293450 |
| Sequence 113 | AA295872 |
| Sequence 114 | AA300065 |
| Sequence 115 | AA305272 |
| Sequence 116 | AA305566 |
| Sequence 117 | AA305591 |
| Sequence 118 | AA305876 |
| Sequence 119 | AA305954 |
| Sequence 120 | AA306620 |
| Sequence 121 | AA306660 |
| Sequence 122 | AA306692 |
| Sequence 123 | AA306696 |
| Sequence 124 | AA307818 |
| Sequence 125 | AA308126 |
| Sequence 126 | AA308230 |
| Sequence 127 | AA308374 |
| Sequence 128 | AA308443 |
| Sequence 129 | AA308570 |
| Sequence 130 | AA308801 |
| Sequence 131 | AA309594 |
| Sequence 132 | AA309832 |
| Sequence 133 | AA311573 |
| Sequence 134 | AA311896 |
| Sequence 135 | AA312002 |
| Sequence 136 | AA313534 |
| Sequence 137 | AA313688 |
| Sequence 138 | AA314188 |
| Sequence 139 | AA314196 |
| Sequence 140 | AA314584 |
| Sequence 141 | AA314961 |
| Sequence 142 | AA315889 |
| Sequence 143 | AA318817 |
| Sequence 144 | AA325285 |
| Sequence 145 | AA325809 |
| Sequence 146 | AA333358 |
| Sequence 147 | AA344846 |
| Sequence 148 | AA347752 |
| Sequence 149 | AA348032 |
| Sequence 150 | AA350063 |
| Sequence 151 | AA350719 |
| Sequence 152 | AA354709 |
| Sequence 153 | AA355003 |
| Sequence 154 | AA356654 |
| Sequence 155 | AA359705 |
| Sequence 156 | AA361393 |
| Sequence 157 | AA361953 |
| Sequence 158 | AA362701 |
| Sequence 159 | AA367082 |
| Sequence 160 | AA371964 |
| Sequence 161 | AA375228 |
| Sequence 162 | AA377891 |
| Sequence 163 | AA384315 |
| Sequence 164 | AA384731 |
| Sequence 165 | AA400974 |
| Sequence 166 | AA401759 |
| Sequence 167 | AA411068 |
| Sequence 168 | AA416628 |
| Sequence 169 | AA421632 |
| Sequence 170 | AA424661 |
| Sequence 171 | AA425212 |
| Sequence 172 | AA425260 |
| Sequence 173 | AA425795 |
| Sequence 174 | AA427816 |
| Sequence 175 | AA428014 |
| Sequence 176 | AA428889 |
| Sequence 177 | AA443112 |
| Sequence 178 | AA445966 |
| Sequence 179 | AA447302 |
| Sequence 180 | AA447645 |
| Sequence 181 | AA448559 |
| Sequence 182 | AA449337 |
| Sequence 183 | AA453555 |
| Sequence 184 | AA453632 |
| Sequence 185 | AA453719 |
| Sequence 186 | AA454129 |
| Sequence 187 | AA455807 |
| Sequence 188 | AA458628 |
| Sequence 189 | AA459544 |
| Sequence 190 | AA460383 |
| Sequence 191 | AA460941 |
| Sequence 192 | AA463563 |
| Sequence 193 | AA464471 |
| Sequence 194 | AA467869 |
| Sequence 195 | AA469319 |
| Sequence 196 | AA476568 |
| Sequence 197 | AA476980 |
| Sequence 198 | AA477822 |
| Sequence 199 | AA478382 |
| Sequence 200 | AA478397 |
| Sequence 201 | AA479044 |
| Sequence 202 | AA479490 |
| Sequence 203 | AA480144 |
| Sequence 204 | AA481078 |
| Sequence 205 | AA488519 |
| Sequence 206 | AA493269 |
| Sequence 207 | AA503327 |
| Sequence 208 | AA505810 |
| Sequence 209 | AA506026 |
| Sequence 210 | AA506542 |
| Sequence 211 | AA507244 |
| Sequence 212 | AA514617 |
| Sequence 213 | AA515132 |
| Sequence 214 | AA521134 |
| Sequence 215 | AA521377 |
| Sequence 216 | AA521478 |
| Sequence 217 | AA527168 |
| Sequence 218 | AA527704 |
| Sequence 219 | AA534504 |
| Sequence 220 | AA552146 |
| Sequence 221 | AA557336 |
| Sequence 222 | AA558876 |
| Sequence 223 | AA559209 |
| Sequence 224 | AA572791 |
| Sequence 225 | AA581467 |
| Sequence 226 | AA582612 |
| Sequence 227 | AA587269 |
| Sequence 228 | AA594137 |
| Sequence 229 | AA599533 |
| Sequence 230 | AA609167 |
| Sequence 231 | AA612898 |
| Sequence 232 | AA625833 |
| Sequence 233 | AA626477 |
| Sequence 234 | AA628322 |
| Sequence 235 | AA634277 |
| Sequence 236 | AA639123 |
| Sequence 237 | AA639223 |
| Sequence 238 | AA641277 |
| Sequence 239 | AA643063 |
| Sequence 240 | AA652845 |
| Sequence 241 | AA663986 |
| Sequence 242 | AA679329 |
| Sequence 243 | AA682527 |
| Sequence 244 | AA682861 |
| Sequence 245 | AA687499 |
| Sequence 246 | AA701625 |
| Sequence 247 | AA703094 |
| Sequence 248 | AA703179 |
| Sequence 249 | AA704155 |
| Sequence 250 | AA704856 |
| Sequence 251 | AA705590 |
| Sequence 252 | AA707255 |
| Sequence 253 | AA714838 |
| Sequence 254 | AA716568 |
| Sequence 255 | AA720598 |
| Sequence 256 | AA723130 |
| Sequence 257 | AA766044 |
| Sequence 258 | AA770043 |
| Sequence 259 | AA773324 |
| Sequence 260 | AA773727 |
| Sequence 261 | AA805504 |
| Sequence 262 | AA807426 |
| Sequence 263 | AA808186 |
| Sequence 264 | AA812594 |
| Sequence 265 | AA827097 |
| Sequence 266 | AA829474 |
| Sequence 267 | AA829769 |
| Sequence 268 | AA853584 |
| Sequence 269 | AA854927 |
| Sequence 270 | AA863276 |
| Sequence 271 | AA863446 |
| Sequence 272 | AA868174 |
| Sequence 273 | AA868640 |
| Sequence 274 | AA872126 |
| Sequence 275 | AA877795 |
| Sequence 276 | AA902103 |
| Sequence 277 | AA903253 |
| Sequence 278 | AA917448 |
| Sequence 279 | AA933679 |
| Sequence 280 | AA933684 |
| Sequence 281 | AA938929 |
| Sequence 282 | AA962515 |
| Sequence 283 | AA973392 |
| Sequence 284 | AA974390 |
| Sequence 285 | AA977809 |
| Sequence 286 | AA993601 |
| Sequence 287 | AF017688 |
| Sequence 288 | AF038251 |
| Sequence 289 | AI002420 |
| Sequence 290 | AI005164 |
| Sequence 291 | AI015844 |
| Sequence 292 | AI025782 |
| Sequence 293 | AI028404 |
| Sequence 294 | AI039096 |
| Sequence 295 | AI061159 |
| Sequence 296 | AI061422 |
| Sequence 297 | AI075189 |
| Sequence 298 | AI079233 |
| Sequence 299 | AI097371 |
| Sequence 300 | AI125755 |
| Sequence 301 | AI142257 |
| Sequence 302 | AI143987 |
| Sequence 303 | AI147744 |
| Sequence 304 | AI148558 |
| Sequence 305 | AI150088 |
| Sequence 306 | AI186525 |
| Sequence 307 | AI199904 |
| Sequence 308 | AI199936 |
| Sequence 309 | AI201570 |
| Sequence 310 | AI201576 |
| Sequence 311 | AI203647 |
| Sequence 312 | AI214250 |
| Sequence 313 | AI216862 |
| Sequence 314 | AI239435 |
| Sequence 315 | AI241369 |
| Sequence 316 | AI245345 |
| Sequence 317 | AI250290 |
| Sequence 318 | AI266663 |
| Sequence 319 | AI267185 |
| Sequence 320 | AI267425 |
| Sequence 321 | AI267612 |
| Sequence 322 | AI275090 |
| Sequence 323 | AI275233 |
| Sequence 324 | AI278769 |
| Sequence 325 | AI278776 |
| Sequence 326 | AI279562 |
| Sequence 327 | AI290088 |
| Sequence 328 | AI290518 |
| Sequence 329 | AI298973 |
| Sequence 330 | AI301926 |
| Sequence 331 | AI307771 |
| Sequence 332 | AI308800 |
| Sequence 333 | AI312603 |
| Sequence 334 | AI342411 |
| Sequence 335 | AI347826 |
| Sequence 336 | AI347995 |
| Sequence 337 | AI349645 |
| Sequence 338 | AI350809 |
| Sequence 339 | AI362793 |
| Sequence 340 | AI366549 |
| Sequence 341 | AI366974 |
| Sequence 342 | AI375141 |
| Sequence 343 | AI376613 |
| Sequence 344 | AI376640 |
| Sequence 345 | AI379614 |
| Sequence 346 | AI400282 |
| Sequence 347 | AI421827 |
| Sequence 348 | AI433157 |
| Sequence 349 | AI436202 |
| Sequence 350 | AI469427 |
| Sequence 351 | AI475758 |
| Sequence 352 | AI525199 |
| Sequence 353 | AI525579 |
| Sequence 354 | AI568908 |
| Sequence 355 | AI620381 |
| Sequence 356 | AI650799 |
| Sequence 357 | Al660145 |
| Sequence 358 | AI670903 |
| Sequence 359 | AI684386 |
| Sequence 360 | AI702062 |
| Sequence 361 | AI743061 |
| Sequence 362 | AL047763 |
| Sequence 363 | C03852 |
| Sequence 364 | C06289 |
| Sequence 365 | D54540 |
| Sequence 366 | D58432 |
| Sequence 367 | D82436 |
| Sequence 368 | F22086 |
| Sequence 369 | F27502 |
| Sequence 370 | H05168 |
| Sequence 371 | H08641 |
| Sequence 372 | H11991 |
| Sequence 373 | H13883 |
| Sequence 374 | H15247 |
| Sequence 375 | H24046 |
| Sequence 376 | H49462 |
| Sequence 377 | H52955 |
| Sequence 378 | H73244 |
| Sequence 379 | H84532 |
| Sequence 380 | H87703 |
| Sequence 381 | H95493 |
| Sequence 382 | N28826 |
| Sequence 383 | N34398 |
| Sequence 384 | N78296 |
| Sequence 385 | R45371 |
| Sequence 386 | P55887 |
| Sequence 387 | R59147 |
| Sequence 388 | R63481 |
| Sequence 389 | R73183 |
| Sequence 390 | P76057 |
| Sequence 391 | R88149 |
| Sequence 392 | R91325 |
| Sequence 393 | T24595 |
| Sequence 394 | T29724 |
| Sequence 395 | T30494 |
| Sequence 396 | T35410 |
| Sequence 397 | T84755 |
| Sequence 398 | W05639 |
| Sequence 399 | W19700 |
| Sequence 400 | W24250 |
| Sequence 401 | W39447 |
| Sequence 402 | W45419 |
| Sequence 403 | W52961 |
| Sequence 404 | W60209 |
| Sequence 405 | Z21669 |
| Sequence 406 | Z25220 |
| Sequence 407 | Z45221 |
| Sequence 408 | AB002368 |
| Sequence 409 | AB018315 |
| Sequence 410 | AB018346 |
| Sequence 411 | AF026548 |
| Sequence 412 | AF037439 |
| Sequence 413 | AF038650 |
| Sequence 414 | AF064491 |
| Sequence 415 | AF070598 |
| Sequence 416 | AF081280 |
| Sequence 417 | AF086336 |
| Sequence 418 | AF131826 |
| Sequence 419 | AL021683 |
| Sequence 420 | D00422 |
| Sequence 421 | D26181 |
| Sequence 422 | D84307 |
| Sequence 423 | D88532 |
| Sequence 424 | L40391 |
| Sequence 425 | M28211 |
| Sequence 426 | U01184 |
| Sequence 427 | U14658 |
| Sequence 428 | U40282 |
| Sequence 429 | U71598 |
| Sequence 430 | U94855 |
| Sequence 431 | Q32353 |
| Sequence 432 | Q32355 |
| Sequence 433 | Q46540 |
| Sequence 434 | T47520 |
| Sequence 435 | V59657 |
| Sequence 436 | X19548 |

TABLE 2

| | |
|---|---|
| Sequence ID | Accession # |

1/56

| | |
|---|---|
| Sequence 1 | A1039871 |
| Sequence 2 | AA778432 |
| Sequence 3 | Al092886 |
| Sequence 4 | AA082314 |
| Sequence 5 | AA862355 |
| Sequence 6 | Al659859 |
| Sequence 7 | AL047713 |
| Sequence 8 | Al189911 |
| Sequence 9 | AA853117 |
| Sequence 10 | AA424894 |
| Sequence 11 | Al366549 |
| Sequence 12 | AA558957 |
| Sequence 13 | AA313534 |
| Sequence 14 | Al139211 |
| Sequence 15 | Al131084 |
| Sequence 16 | AA601051 |
| Sequence 17 | Al267615 |
| Sequence 18 | R71549 |
| Sequence 19 | Al373754 |
| Sequence 20 | AA609618 |
| Sequence 21 | AA122401 |
| Sequence 22 | AA335862 |
| Sequence 23 | Al085616 |
| Sequence 24 | Al291072 |
| Sequence 25 | AA206225 |
| Sequence 26 | Al654989 |
| Sequence 27 | AA160532 |
| Sequence 28 | AA451923 |
| Sequence 29 | AA022750 |
| Sequence 30 | AA299694 |
| Sequence 31 | AA292009 |
| Sequence 32 | AA476770 |
| Sequence 33 | AA436859 |
| Sequence 34 | AA706420 |
| Sequence 35 | AA923755 |
| Sequence 36 | AA923755 |
| Sequence 37 | AA100982 |
| Sequence 38 | AA470067 |
| Sequence 39 | AA418433 |
| Sequence 40 | AA126150 |
| Sequence 41 | AA151805 |
| Sequence 42 | AA393176 |
| Sequence 43 | AA134909 |
| Sequence 44 | W29031 |
| Sequence 45 | AA482432 |
| Sequence 46 | AA115271 |
| Sequence 47 | AA219390 |
| Sequence 48 | AA232172 |
| Sequence 49 | AA159064 |
| Sequence 50 | AA358940 |
| Sequence 51 | AA041369 |
| Sequence 52 | AA063638 |
| Sequence 53 | AA233221 |
| Sequence 54 | AA609837 |
| Sequence 55 | AA130076 |
| Sequence 56 | AA307735 |
| Sequence 57 | AA063638 |

2/56

| | |
|---|---|
| Sequence 58 | AA032275 |
| Sequence 59 | AA437301 |
| Sequence 60 | AA977661 |
| Sequence 61 | AA977661 |
| Sequence 62 | AA436315 |
| Sequence 63 | AA453784 |
| Sequence 64 | Al282538 |
| Sequence 65 | AA335551 |
| Sequence 66 | AA335977 |
| Sequence 67 | Z44898 |
| Sequence 68 | AA186404 |
| Sequence 69 | AA845426 |
| Sequence 70 | AA406233 |
| Sequence 71 | AA026625 |
| Sequence 72 | AA323696 |
| Sequence 73 | Al084071 |
| Sequence 74 | Al084071 |
| Sequence 75 | AA308585 |
| Sequence 76 | H96671 |
| Sequence 77 | Al082695 |
| Sequence 78 | AA215319 |
| Sequence 79 | AA047729 |
| Sequence 80 | AA437301 |
| Sequence 81 | AA081269 |
| Sequence 82 | AA148523 |
| Sequence 83 | AA148523 |
| Sequence 84 | AA054564 |
| Sequence 85 | AA037260 |
| Sequence 86 | U89188 |
| Sequence 87 | AA307058 |
| Sequence 88 | AA486182 |
| Sequence 89 | Al347167 |
| Sequence 90 | AA034975 |
| Sequence 91 | AA461052 |
| Sequence 92 | W31059 |
| Sequence 93 | AA576065 |
| Sequence 94 | Al346050 |
| Sequence 95 | Z44898 |
| Sequence 96 | U69188 |
| Sequence 97 | W90000 |
| Sequence 98 | Al267379 |
| Sequence 99 | AA429418 |
| Sequence 100 | AA152275 |
| Sequence 101 | AA609837 |
| Sequence 102 | AA037260 |
| Sequence 103 | AA121249 |
| Sequence 104 | AA133375 |
| Sequence 105 | AA081204 |
| Sequence 106 | AA401204 |
| Sequence 107 | AA063074 |
| Sequence 108 | Al148825 |
| Sequence 109 | AA127645 |
| Sequence 110 | AA025006 |
| Sequence 111 | Z44898 |
| Sequence 112 | AA194362 |
| Sequence 113 | AA515031 |
| Sequence 114 | AA429418 |

3/56

| | |
|---|---|
| Sequence 115 | Al148825 |
| Sequence 116 | W27631 |
| Sequence 117 | AA373030 |
| Sequence 118 | Al628638 |
| Sequence 119 | AA831467 |
| Sequence 120 | AA443231 |
| Sequence 121 | AA443231 |
| Sequence 122 | Al308800 |
| Sequence 123 | AA857509 |
| Sequence 124 | AA632557 |
| Sequence 125 | AA150653 |
| Sequence 126 | AA148885 |
| Sequence 127 | AA318422 |
| Sequence 128 | Al580104 |
| Sequence 129 | AA227945 |
| Sequence 130 | AA429418 |
| Sequence 131 | AA428362 |
| Sequence 132 | AA229611 |
| Sequence 133 | Al261664 |
| Sequence 134 | AA788933 |
| Sequence 135 | AA504389 |
| Sequence 136 | AA788933 |
| Sequence 137 | AA233122 |
| Sequence 138 | AA192168 |
| Sequence 139 | AA452930 |
| Sequence 140 | AA523928 |
| Sequence 141 | W27631 |
| Sequence 142 | R14684 |
| Sequence 143 | AA452335 |
| Sequence 144 | AA291003 |
| Sequence 145 | AA443231 |
| Sequence 146 | AA443231 |
| Sequence 147 | Al241358 |
| Sequence 148 | AA070540 |
| Sequence 149 | AA581144 |
| Sequence 150 | AA581144 |
| Sequence 151 | AA928420 |
| Sequence 152 | AA291003 |
| Sequence 153 | AA227124 |
| Sequence 154 | AA969067 |
| Sequence 155 | AA760854 |
| Sequence 156 | AA580691 |
| Sequence 157 | AA770300 |
| Sequence 158 | AA770300 |
| Sequence 159 | AA083836 |
| Sequence 160 | AA045464 |
| Sequence 161 | AA167325 |
| Sequence 162 | AA115676 |
| Sequence 163 | AA187741 |
| Sequence 164 | AA352347 |
| Sequence 165 | AA375384 |
| Sequence 166 | AA136215 |
| Sequence 167 | AA318422 |
| Sequence 168 | AA244162 |
| Sequence 169 | AA211509 |
| Sequence 170 | AA430507 |
| Sequence 171 | AA515132 |

4/56

| | |
|---|---|
| Sequence 172 | AA284355 |
| Sequence 173 | AA167246 |
| Sequence 174 | AA083836 |
| Sequence 175 | AA113359 |
| Sequence 176 | AA669126 |
| Sequence 177 | Al683279 |
| Sequence 178 | AA296452 |
| Sequence 179 | AA765577 |
| Sequence 180 | AA086276 |
| Sequence 181 | AA214176 |
| Sequence 182 | AA476889 |
| Sequence 183 | Al016888 |
| Sequence 184 | AA600214 |
| Sequence 185 | AA375384 |
| Sequence 186 | AA453784 |
| Sequence 187 | AA453784 |
| Sequence 188 | W76487 |
| Sequence 189 | AA247690 |
| Sequence 190 | AA737182 |
| Sequence 191 | AA129463 |
| Sequence 192 | AA129463 |
| Sequence 193 | H04522 |
| Sequence 194 | AA169457 |
| Sequence 195 | AA010849 |
| Sequence 196 | AA156616 |
| Sequence 197 | AA431321 |
| Sequence 198 | AA558281 |
| Sequence 199 | AA581144 |
| Sequence 200 | AA935526 |
| Sequence 201 | AA088373 |
| Sequence 202 | AA453445 |
| Sequence 203 | AA968726 |
| Sequence 204 | AA770537 |
| Sequence 205 | AA330007 |
| Sequence 206 | AA330007 |
| Sequence 207 | AA853539 |
| Sequence 208 | T30659 |
| Sequence 209 | AA501373 |
| Sequence 210 | AA935526 |
| Sequence 211 | Al672524 |
| Sequence 212 | Al274797 |
| Sequence 213 | Al129827 |
| Sequence 214 | Al478372 |
| Sequence 215 | AA665560 |
| Sequence 216 | AA788933 |
| Sequence 217 | AA161518 |
| Sequence 218 | AA041467 |
| Sequence 219 | Al074333 |
| Sequence 220 | AA044787 |
| Sequence 221 | AA825917 |
| Sequence 222 | AA825917 |
| Sequence 223 | F27264 |
| Sequence 224 | H97360 |
| Sequence 225 | AA313688 |
| Sequence 226 | AA024982 |
| Sequence 227 | Al693441 |
| Sequence 228 | Al351167 |

5/56

| | |
|---|---|
| Sequence 229 | AA398886 |
| Sequence 230 | AA621740 |
| Sequence 231 | Al539679 |
| Sequence 232 | AA426083 |
| Sequence 233 | AA399022 |
| Sequence 234 | AA412184 |
| Sequence 235 | AA788933 |
| Sequence 236 | AA376374 |
| Sequence 237 | Al690181 |
| Sequence 238 | AA213902 |
| Sequence 239 | AA514564 |
| Sequence 240 | AA701870 |
| Sequence 241 | AA486954 |
| Sequence 242 | AA284462 |
| Sequence 243 | AA465528 |
| Sequence 244 | AA171834 |
| Sequence 245 | Al081515 |
| Sequence 246 | Al267532 |
| Sequence 247 | AL048644 |
| Sequence 248 | AA281739 |
| Sequence 249 | AA570181 |
| Sequence 250 | Al660919 |
| Sequence 251 | AA088764 |
| Sequence 252 | AA903285 |
| Sequence 253 | AA283080 |
| Sequence 254 | AA465285 |
| Sequence 255 | AA159916 |
| Sequence 256 | AA026215 |
| Sequence 257 | AA043908 |
| Sequence 258 | AA375325 |
| Sequence 259 | Al052317 |
| Sequence 260 | AA194535 |
| Sequence 261 | AA846439 |
| Sequence 262 | AA492032 |
| Sequence 263 | Al026767 |
| Sequence 264 | Al004557 |
| Sequence 265 | AA335502 |
| Sequence 266 | AA001794 |
| Sequence 267 | AA333713 |
| Sequence 268 | AA730829 |
| Sequence 269 | AA127626 |
| Sequence 270 | AA101212 |
| Sequence 271 | AA303085 |
| Sequence 272 | Al673189 |
| Sequence 273 | Al086254 |
| Sequence 274 | AA035773 |
| Sequence 275 | AA916110 |
| Sequence 276 | AA166874 |
| Sequence 277 | AA422177 |
| Sequence 278 | AA262878 |
| Sequence 279 | AA282134 |
| Sequence 280 | AA156237 |
| Sequence 281 | AA425378 |
| Sequence 282 | AA150409 |
| Sequence 283 | AA045417 |
| Sequence 284 | Al052317 |
| Sequence 285 | W01842 |

6/56

| | |
|---|---|
| Sequence 286 | AA425292 |
| Sequence 287 | AA985622 |
| Sequence 288 | AA722855 |
| Sequence 289 | AA426216 |
| Sequence 290 | AA788933 |
| Sequence 291 | Al138898 |
| Sequence 292 | AA778250 |
| Sequence 293 | W78198 |
| Sequence 294 | AA788933 |
| Sequence 295 | Al342607 |
| Sequence 296 | AA426216 |
| Sequence 297 | AA927120 |
| Sequence 298 | AA788933 |
| Sequence 299 | Al079871 |
| Sequence 300 | Al741829 |
| Sequence 301 | AA418715 |
| Sequence 302 | AA150636 |
| Sequence 303 | Al433965 |
| Sequence 304 | AA088373 |
| Sequence 305 | AA471070 |
| Sequence 306 | Al673422 |
| Sequence 307 | AA203123 |
| Sequence 308 | AA203627 |
| Sequence 309 | AA193592 |
| Sequence 310 | AA206064 |
| Sequence 311 | R31249 |
| Sequence 312 | AA783933 |
| Sequence 313 | AA282369 |
| Sequence 314 | AA149594 |
| Sequence 315 | AA347635 |
| Sequence 316 | AA293172 |
| Sequence 317 | AA832167 |
| Sequence 318 | AA053133 |
| Sequence 319 | AA908731 |
| Sequence 320 | AA808830 |
| Sequence 321 | AA779747 |
| Sequence 322 | AA084038 |
| Sequence 323 | Al365500 |
| Sequence 324 | Al609252 |
| Sequence 325 | H08650 |
| Sequence 328 | H92511 |
| Sequence 327 | AA088373 |
| Sequence 328 | H66289 |
| Sequence 329 | W73086 |
| Sequence 330 | AA035773 |
| Sequence 331 | AA481432 |
| Sequence 332 | AA082084 |
| Sequence 333 | AA490665 |
| Sequence 334 | AA429541 |
| Sequence 335 | AA568231 |
| Sequence 336 | AA426216 |
| Sequence 337 | AA738430 |
| Sequence 338 | AA627132 |
| Sequence 339 | AA402714 |
| Sequence 340 | AA025585 |
| Sequence 341 | AA493305 |
| Sequence 342 | AA278542 |

7/56

| | |
|---|---|
| Sequence 343 | AA278542 |
| Sequence 344 | AA442936 |
| Sequence 345 | AA779747 |
| Sequence 346 | Al283155 |
| Sequence 347 | Al263446 |
| Sequence 348 | AA013042 |
| Sequence 349 | AA081426 |
| Sequence 350 | AA484061 |
| Sequence 351 | AA315535 |
| Sequence 352 | AA399022 |
| Sequence 353 | Al127825 |
| Sequence 354 | AA715828 |
| Sequence 355 | Al243931 |
| Sequence 356 | Al024753 |
| Sequence 357 | AA086070 |
| Sequence 358 | AA625159 |
| Sequence 359 | AA426216 |
| Sequence 360 | AA426216 |
| Sequence 361 | Al628734 |
| Sequence 362 | Al358846 |
| Sequence 363 | AA013042 |
| Sequence 364 | Al273856 |
| Sequence 365 | AA493647 |
| Sequence 366 | AA450075 |
| Sequence 367 | AA115777 |
| Sequence 368 | AA494342 |
| Sequence 369 | AA181331 |
| Sequence 370 | AA418715 |
| Sequence 371 | AA662648 |
| Sequence 372 | AA086070 |
| Sequence 373 | AA487250 |
| Sequence 374 | AA099101 |
| Sequence 375 | AA093957 |
| Sequence 376 | AA136215 |
| Sequence 377 | AA256459 |
| Sequence 378 | Al760421 |
| Sequence 379 | AA082675 |
| Sequence 380 | AA180746 |
| Sequence 381 | AA534365 |
| Sequence 382 | W28567 |
| Sequence 383 | AA069657 |
| Sequence 384 | AA178880 |
| Sequence 385 | H23090 |
| Sequence 386 | AA130285 |
| Sequence 387 | Al052317 |
| Sequence 388 | AA453784 |
| Sequence 389 | AA312917 |
| Sequence 390 | AA521024 |
| Sequence 391 | AA188744 |
| Sequence 392 | AA593011 |
| Sequence 393 | N78223 |
| Sequence 394 | AA669106 |
| Sequence 395 | AA548445 |
| Sequence 396 | AA046827 |
| Sequence 397 | AA618310 |
| Sequence 398 | AA426216 |
| Sequence 399 | AA281412 |

8/56

| | |
|---|---|
| Sequence 400 | AA532835 |
| Sequence 401 | AA809070 |
| Sequence 402 | Al608739 |
| Sequence 403 | AA977802 |
| Sequence 404 | H68725 |
| Sequence 405 | AA125809 |
| Sequence 406 | Al299874 |
| Sequence 407 | AA453784 |
| Sequence 408 | AA101236 |
| Sequence 409 | AA491219 |
| Sequence 410 | AA149853 |
| Sequence 411 | AA151345 |
| Sequence 412 | Al273856 |
| Sequence 413 | AA405425 |
| Sequence 414 | AA599864 |
| Sequence 415 | AA411436 |
| Sequence 416 | AA131439 |
| Sequence 417 | AA706347 |
| Sequence 418 | Al349772 |
| Sequence 419 | AA488463 |
| Sequence 420 | AA083482 |
| Sequence 421 | AA083482 |
| Sequence 422 | AA149853 |
| Sequence 423 | AA255613 |
| Sequence 424 | AA627448 |
| Sequence 425 | AA453784 |
| Sequence 426 | AA022788 |
| Sequence 427 | AA635003 |
| Sequence 428 | AA576419 |
| Sequence 429 | AA459880 |
| Sequence 430 | AA131053 |
| Sequence 431 | AA496640 |
| Sequence 432 | Al582629 |
| Sequence 433 | Al582629 |
| Sequence 434 | Al023912 |
| Sequence 435 | AA977802 |
| Sequence 436 | W00823 |
| Sequence 437 | W00823 |
| Sequence 438 | Al674397 |
| Sequence 439 | AA700350 |
| Sequence 440 | AA745614 |
| Sequence 441 | Al302190 |
| Sequence 442 | AA393518 |
| Sequence 443 | N27985 |
| Sequence 444 | AA468753 |
| Sequence 445 | AA653775 |
| Sequence 446 | AA653775 |
| Sequence 447 | AA088373 |
| Sequence 448 | AA088373 |
| Sequence 449 | AA130783 |
| Sequence 450 | AA461143 |
| Sequence 451 | R78778 |
| Sequence 452 | AA031550 |
| Sequence 453 | AA461143 |
| Sequence 454 | AA303484 |
| Sequence 455 | AA301297 |
| Sequence 456 | AA744503 |

9/56

| | |
|---|---|
| Sequence 457 | T67813 |
| Sequence 458 | Al564319 |
| Sequence 459 | Al564319 |
| Sequence 460 | Al138898 |
| Sequence 461 | Al138898 |
| Sequence 462 | AA301297 |
| Sequence 463 | Al302436 |
| Sequence 464 | H27251 |
| Sequence 465 | AA418715 |
| Sequence 466 | 001749 |
| Sequence 467 | AA083471 |
| Sequence 468 | Al264186 |
| Sequence 469 | T66196 |
| Sequence 470 | H29445 |
| Sequence 471 | Al469093 |
| Sequence 472 | AA136215 |
| Sequence 473 | Al267185 |
| Sequence 474 | AA935526 |
| Sequence 475 | AA026157 |
| Sequence 476 | AA001277 |
| Sequence 477 | AA552588 |
| Sequence 478 | AA099921 |
| Sequence 479 | AA380495 |
| Sequence 480 | R77384 |
| Sequence 481 | AA024924 |
| Sequence 482 | Al309007 |
| Sequence 483 | AA278434 |
| Sequence 484 | AA303150 |
| Sequence 485 | AA460060 |
| Sequence 486 | AA169355 |
| Sequence 487 | Al201573 |
| Sequence 488 | AA779747 |
| Sequence 489 | AA468753 |
| Sequence 490 | AA091431 |
| Sequence 491 | AA464646 |
| Sequence 492 | AA083836 |
| Sequence 493 | AA151506 |
| Sequence 494 | AA069195 |
| Sequence 495 | AA007137 |
| Sequence 496 | AA178912 |
| Sequence 497 | AA225164 |
| Sequence 498 | Al565125 |
| Sequence 499 | AA483181 |
| Sequence 500 | AA470909 |
| Sequence 501 | AA650493 |
| Sequence 502 | AA059309 |
| Sequence 503 | AA677142 |
| Sequence 504 | AA447503 |
| Sequence 505 | AA252216 |
| Sequence 506 | F33257 |
| Sequence 507 | AA576667 |
| Sequence 508 | AA320124 |
| Sequence 509 | AA082569 |
| Sequence 510 | AA573200 |
| Sequence 511 | AA178880 |
| Sequence 512 | AA176433 |
| Sequence 513 | AA788933 |

10/56

| | |
|---|---|
| Sequence 514 | Al033037 |
| Sequence 515 | AA533283 |
| Sequence 516 | Al267185 |
| Sequence 517 | AA280123 |
| Sequence 518 | C17183 |
| Sequence 519 | AA779747 |
| Sequence 520 | Al283155 |
| Sequence 521 | AA308736 |
| Sequence 522 | H52011 |
| Sequence 523 | AA034975 |
| Sequence 524 | AA034908 |
| Sequence 525 | AA564296 |
| Sequence 526 | AA515031 |
| Sequence 527 | AA083471 |
| Sequence 528 | AA088373 |
| Sequence 529 | AA088373 |
| Sequence 530 | Al218204 |
| Sequence 531 | N46402 |
| Sequence 532 | AA099023 |
| Sequence 533 | AA600987 |
| Sequence 534 | Al267185 |
| Sequence 535 | N46402 |
| Sequence 536 | N46402 |
| Sequence 537 | AA131053 |
| Sequence 538 | R19004 |
| Sequence 539 | AA099878 |
| Sequence 540 | Al608720 |
| Sequence 541 | Al131214 |
| Sequence 542 | Al139097 |
| Sequence 543 | AA089857 |
| Sequence 544 | AA047849 |
| Sequence 545 | AA058822 |
| Sequence 546 | AA147978 |
| Sequence 547 | AA199850 |
| Sequence 548 | Al167659 |
| Sequence 549 | Al167659 |
| Sequence 550 | AA574054 |
| Sequence 551 | W37586 |
| Sequence 552 | AA088373 |
| Sequence 553 | AA157619 |
| Sequence 554 | AA013042 |
| Sequence 555 | AA300170 |
| Sequence 556 | AA430088 |
| Sequence 557 | AA136096 |
| Sequence 558 | AA136756 |
| Sequence 559 | W07393 |
| Sequence 560 | AA143577 |
| Sequence 561 | AA642766 |
| Sequence 562 | AA554920 |
| Sequence 563 | AA452335 |
| Sequence 564 | AA532484 |
| Sequence 565 | AA609837 |
| Sequence 566 | AA152275 |
| Sequence 567 | Al192851 |
| Sequence 568 | AA228082 |
| Sequence 569 | Al093501 |
| Sequence 570 | AA905277 |

11/56

| | |
|---|---|
| Sequence 571 | AA465301 |
| Sequence 572 | AA172248 |
| Sequence 573 | AA129819 |
| Sequence 574 | AA428120 |
| Sequence 575 | Al291105 |
| Sequence 576 | Al750962 |
| Sequence 577 | AA011024 |
| Sequence 578 | AA873159 |
| Sequence 579 | AA873159 |
| Sequence 580 | AA846266 |
| Sequence 581 | AA652080 |
| Sequence 582 | Al339481 |
| Sequence 583 | AA609172 |
| Sequence 584 | AA609172 |
| Sequence 585 | AA908731 |
| Sequence 586 | Al147200 |
| Sequence 587 | AA864497 |
| Sequence 588 | AA083482 |
| Sequence 589 | Al279718 |
| Sequence 590 | AA188375 |
| Sequence 591 | AA088373 |
| Sequence 592 | AA280110 |
| Sequence 593 | AA744712 |
| Sequence 594 | AA936089 |
| Sequence 595 | AA076368 |
| Sequence 596 | AA774663 |
| Sequence 597 | AA527389 |
| Sequence 598 | AA007495 |
| Sequence 599 | AA045264 |
| Sequence 600 | AA523252 |
| Sequence 601 | AA552588 |
| Sequence 602 | AA173924 |
| Sequence 603 | AA465301 |
| Sequence 604 | AA599533 |
| Sequence 605 | AA614105 |
| Sequence 606 | AA384078 |
| Sequence 607 | AA872507 |
| Sequence 608 | AA045936 |
| Sequence 609 | AA081269 |
| Sequence 610 | AA572758 |
| Sequence 611 | AA007271 |
| Sequence 612 | Z78349 |
| Sequence 613 | AA626469 |
| Sequence 614 | AA551215 |
| Sequence 615 | AA621740 |
| Sequence 616 | AA304249 |
| Sequence 617 | AA877288 |
| Sequence 618 | AA483182 |
| Sequence 619 | N78223 |
| Sequence 620 | N78268 |
| Sequence 621 | AA365398 |
| Sequence 622 | AA131231 |
| Sequence 623 | P55380 |
| Sequence 624 | Al078529 |
| Sequence 625 | Al767123 |
| Sequence 626 | Al700226 |
| Sequence 627 | AA487586 |

12/56

| | |
|---|---|
| Sequence 628 | Al033304 |
| Sequence 629 | AA436895 |
| Sequence 630 | W01842 |
| Sequence 631 | AA864853 |
| Sequence 632 | AA444134 |
| Sequence 633 | Al700226 |
| Sequence 634 | AA853130 |
| Sequence 635 | H52011 |
| Sequence 636 | AA618484 |
| Sequence 637 | AA101484 |
| Sequence 638 | AA649746 |
| Sequence 639 | H01401 |
| Sequence 640 | AA375933 |
| Sequence 641 | AA166905 |
| Sequence 642 | AA151918 |
| Sequence 643 | AA845573 |
| Sequence 644 | AA013363 |
| Sequence 645 | Al096880 |
| Sequence 646 | AA905781 |
| Sequence 647 | Al052317 |
| Sequence 648 | AA513827 |
| Sequence 649 | Al393144 |
| Sequence 650 | AA788933 |
| Sequence 651 | AA788933 |
| Sequence 652 | AA311098 |
| Sequence 653 | AA542829 |
| Sequence 654 | AA166622 |
| Sequence 655 | Al054163 |
| Sequence 656 | AA164243 |
| Sequence 657 | Al288783 |
| Sequence 658 | Al186605 |
| Sequence 659 | C18682 |
| Sequence 660 | AA340224 |
| Sequence 661 | AA171834 |
| Sequence 662 | AA196793 |
| Sequence 663 | Al423079 |
| Sequence 664 | Al423079 |
| Sequence 665 | A1521128 |
| Sequence 666 | AA526226 |
| Sequence 667 | AA165306 |
| Sequence 668 | AA878771 |
| Sequence 669 | AA195865 |
| Sequence 670 | W16508 |
| Sequence 671 | AA995597 |
| Sequence 672 | Al042002 |
| Sequence 673 | AA936566 |
| Sequence 674 | AA936566 |
| Sequence 675 | AA977802 |
| Sequence 676 | AA913590 |
| Sequence 677 | AA913590 |
| Sequence 678 | Al360219 |
| Sequence 679 | AA804436 |
| Sequence 680 | AA548082 |
| Sequence 681 | T66196 |
| Sequence 682 | AA199802 |
| Sequence 683 | Al040294 |
| Sequence 684 | AA402901 |

13/56

| | |
|---|---|
| Sequence 685 | AA426653 |
| Sequence 686 | AA001794 |
| Sequence 687 | AA514564 |
| Sequence 688 | D54004 |
| Sequence 689 | AA211272 |
| Sequence 690 | AA161003 |
| Sequence 691 | AA232705 |
| Sequence 692 | AA020855 |
| Sequence 693 | AA081395 |
| Sequence 694 | AA885514 |
| Sequence 695 | T71404 |
| Sequence 696 | W01842 |
| Sequence 697 | AA094609 |
| Sequence 698 | AA062957 |
| Sequence 699 | AA026215 |
| Sequence 700 | AA158724 |
| Sequence 701 | Al243555 |
| Sequence 702 | AA026719 |
| Sequence 703 | AA036944 |
| Sequence 704 | AA076262 |
| Sequence 705 | AA120908 |
| Sequence 706 | AA161066 |
| Sequence 707 | Al391479 |
| Sequence 708 | AA343324 |
| Sequence 709 | N77582 |
| Sequence 710 | AA083471 |
| Sequence 711 | AA872507 |
| Sequence 712 | Al093740 |
| Sequence 713 | AA731306 |
| Sequence 714 | AA305494 |
| Sequence 715 | AA699927 |
| Sequence 716 | AA701448 |
| Sequence 717 | Al554245 |
| Sequence 718 | AA132605 |
| Sequence 719 | AA825917 |
| Sequence 720 | W78198 |
| Sequence 721 | Al609252 |
| Sequence 722 | AA022788 |
| Sequence 723 | AA026625 |
| Sequence 724 | AA206519 |
| Sequence 725 | AA256335 |
| Sequence 726 | AA256335 |
| Sequence 727 | AA013363 |
| Sequence 728 | Al300590 |
| Sequence 729 | AA069733 |
| Sequence 730 | AA182841 |
| Sequence 731 | Al342480 |
| Sequence 732 | AA451894 |
| Sequence 733 | AA915959 |
| Sequence 734 | AA147833 |
| Sequence 735 | AA229993 |
| Sequence 736 | AA199821 |
| Sequence 737 | Al198431 |
| Sequence 738 | AA928420 |
| Sequence 739 | AA256320 |
| Sequence 740 | Al267185 |
| Sequence 741 | AA528456 |

14/56

| | |
|---|---|
| Sequence 742 | Al567965 |
| Sequence 743 | AA101299 |
| Sequence 744 | AA618484 |
| Sequence 745 | Al278872 |
| Sequence 746 | Al278872 |
| Sequence 747 | AA708712 |
| Sequence 748 | AA398892 |
| Sequence 749 | N52168 |
| Sequence 750 | AA827758 |
| Sequence 751 | R56747 |
| Sequence 752 | AA854014 |
| Sequence 753 | AA904266 |
| Sequence 754 | AA770557 |
| Sequence 755 | AL048446 |
| Sequence 756 | AA062817 |
| Sequence 757 | AA464646 |
| Sequence 758 | AA323097 |
| Sequence 759 | AA102168 |
| Sequence 760 | AA528121 |
| Sequence 761 | AA526498 |
| Sequence 762 | AA877213 |
| Sequence 763 | AA864690 |
| Sequence 764 | AA864690 |
| Sequence 765 | AA788933 |
| Sequence 766 | AA235835 |
| Sequence 767 | AA558066 |
| Sequence 768 | AA149151 |
| Sequence 769 | AA583526 |
| Sequence 770 | AA013363 |
| Sequence 771 | AA013363 |
| Sequence 772 | Al141567 |
| Sequence 773 | AA187406 |
| Sequence 774 | Al499986 |
| Sequence 775 | AA488398 |
| Sequence 776 | AA418408 |
| Sequence 777 | AA418408 |
| Sequence 778 | AA147312 |
| Sequence 779 | AA581130 |
| Sequence 780 | AA582851 |
| Sequence 781 | AA075393 |
| Sequence 782 | AA737562 |
| Sequence 783 | Al538172 |
| Sequence 784 | N57174 |
| Sequence 785 | AA453479 |
| Sequence 786A | A442561 |
| Sequence 787 | AA448973 |
| Sequence 788 | Al079871 |
| Sequence 789 | AA487467 |
| Sequence 790 | AA442948 |
| Sequence 791 | AA078387 |
| Sequence 792 | AA620980 |
| Sequence 793 | AA195128 |
| Sequence 794 | Al126689 |
| Sequence 795 | AA446578 |
| Sequence 796 | AA649064 |
| Sequence 797 | AA938757 |
| Sequence 798 | AA806532 |

15/56

| | |
|---|---|
| Sequence 799 | AA316566 |
| Sequence 800 | H98646 |
| Sequence 801 | AA635393 |
| Sequence 802 | AA191045 |
| Sequence 803 | Al198431 |
| Sequence 804 | AA595562 |
| Sequence 805 | AA532383 |
| Sequence 806 | AA430059 |
| Sequence 807 | AA609837 |
| Sequence 808 | AA126418 |
| Sequence 809 | Al083558 |
| Sequence 810 | AA465584 |
| Sequence 811 | Al129865 |
| Sequence 812 | AA316089 |
| Sequence 813 | AA341011 |
| Sequence 814 | W60762 |
| Sequence 815 | R24022 |
| Sequence 816 | R25322 |
| Sequence 817 | Al249905 |
| Sequence 818 | AA126798 |
| Sequence 819 | AA551607 |
| Sequence 820 | AA418783 |
| Sequence 821 | AA151600 |
| Sequence 822 | AA600173 |
| Sequence 823 | Al245990 |
| Sequence 824 | Al056158 |
| Sequence 825 | AA009816 |
| Sequence 826 | R91618 |
| Sequence 827 | AA977802 |
| Sequence 828 | Al741118 |
| Sequence 829 | AA039810 |
| Sequence 830 | AA357956 |
| Sequence 831 | AA431278 |
| Sequence 832 | Al096925 |
| Sequence 833 | AA400833 |
| Sequence 834 | Al309380 |
| Sequence 835 | AA330640 |
| Sequence 836 | AA169457 |
| Sequence 837 | Al079871 |
| Sequence 838 | AA236339 |
| Sequence 839 | Al301952 |
| Sequence 840 | W03618 |
| Sequence 841 | W03618 |
| Sequence 842 | AA543080 |
| Sequence 843 | AA303085 |
| Sequence 844 | AA779747 |
| Sequence 845 | AA127826 |
| Sequence 846 | AA364229 |
| Sequence 847 | AA364229 |
| Sequence 848 | AA747475 |
| Sequence 849 | F20791 |
| Sequence 850 | F20791 |
| Sequence 851 | AA814278 |
| Sequence 852 | N39195 |
| Sequence 853 | Al700982 |
| Sequence 854 | AA136215 |
| Sequence 855 | Al089782 |

16/56

| | |
|---|---|
| Sequence 856 | AA827570 |
| Sequence 857 | AA983883 |
| Sequence 858 | AA192168 |
| Sequence 859 | Al097410 |
| Sequence 860 | AA742474 |
| Sequence 861 | W69615 |
| Sequence 862 | Al051843 |
| Sequence 863 | R60403 |
| Sequence 864 | AA101010 |
| Sequence 865 | AA932039 |
| Sequence 866 | AA496669 |
| Sequence 867 | AA283707 |
| Sequence 868 | AA088344 |
| Sequence 869 | AA654827 |
| Sequence 870 | Al246125 |
| Sequence 871 | Al079871 |
| Sequence 872 | Al079871 |
| Sequence 873 | N88286 |
| Sequence 874 | Al738508 |
| Sequence 875 | AA070469 |
| Sequence 876 | AA256335 |
| Sequence 877 | AA256335 |
| Sequence 878 | AA010044 |
| Sequence 879 | AA091053 |
| Sequence 880 | AA000981 |
| Sequence 881 | AA148023 |
| Sequence 882 | AA622574 |
| Sequence 883 | AA936960 |
| Sequence 884 | Al201573 |
| Sequence 885 | Al344021 |
| Sequence 886 | Al127179 |
| Sequence 887 | AA026454 |
| Sequence 888 | AA026454 |
| Sequence 889 | AA136756 |
| Sequence 890 | AA129410 |
| Sequence 891 | Al032188 |
| Sequence 892 | Al032188 |
| Sequence 893 | Al741829 |
| Sequence 894 | Al214206 |
| Sequence 895 | AA314222 |
| Sequence 896 | AA099592 |
| Sequence 897 | AA765228 |
| Sequence 898 | AA831729 |
| Sequence 899 | AA190559 |
| Sequence 900 | W01842 |
| Sequence 901 | N31402 |
| Sequence 902 | Al267185 |
| Sequence 903 | AA492465 |
| Sequence 904 | Al611173 |
| Sequence 905 | Al311440 |
| Sequence 906 | AA447503 |
| Sequence 907 | AA412359 |
| Sequence 908 | AA700540 |
| Sequence 909 | Al332755 |
| Sequence 910 | AA115271 |
| Sequence 911 | AA009997 |
| Sequence 912 | AA150445 |

17/56

| | |
|---|---|
| Sequence 913 | AA741015 |
| Sequence 914 | AA397741 |
| Sequence 915 | AA723209 |
| Sequence 916 | AA773607 |
| Sequence 917 | Al309007 |
| Sequence 918 | AA977584 |
| Sequence 919 | AA143311 |
| Sequence 920 | W95589 |
| Sequence 921 | AAS14638 |
| Sequence 922 | Al267148 |
| Sequence 923 | Al311440 |
| Sequence 924 | Al621272 |
| Sequence 925 | Al559470 |
| Sequence 926 | AA325222 |
| Sequence 927 | AA325222 |
| Sequence 928 | Al369447 |
| Sequence 929 | AA773804 |
| Sequence 930 | AA490210 |
| Sequence 931 | AA253384 |
| Sequence 932 | AA652436 |
| Sequence 933 | AA983883 |
| Sequence 934 | AA323560 |
| Sequence 935 | R21821 |
| Sequence 936 | AA182841 |
| Sequence 937 | AA255567 |
| Sequence 938 | AA610706 |
| Sequence 939 | AA306015 |
| Sequence 940A | A565996 |
| Sequence 941 | Al049770 |
| Sequence 942 | AA199644 |
| Sequence 943 | AA224371 |
| Sequence 944 | AA913590 |
| Sequence 945 | AA523526 |
| Sequence 946 | Al144108 |
| Sequence 947 | AA377383 |
| Sequence 948 | AA436438 |
| Sequence 949 | AA788933 |
| Sequence 950 | Al025517 |
| Sequence 951 | AA447250 |
| Sequence 952 | AA722512 |
| Sequence 953 | AA447590 |
| Sequence 954 | AA155915 |
| Sequence 955 | AA412059 |
| Sequence 956 | AA375325 |
| Sequence 957 | AA082684 |
| Sequence 958 | N47128 |
| Sequence 959 | AA227610 |
| Sequence 960 | R85339 |
| Sequence 961 | R85339 |
| Sequence 962 | AA521024 |
| Sequence 963 | AA069611 |
| Sequence 964 | AA303084 |
| Sequence 965 | AA426216 |
| Sequence 966 | AA706730 |
| Sequence 967 | R24799 |
| Sequence 968 | AA129744 |
| Sequence 969 | AA436541 |

18/56

| | | |
|---|---|---|
| Sequence 970 | AA524721 | |
| Sequence 971 | Al492230 | |
| Sequence 972 | AA209340 | |
| Sequence 973 | AA974657 | |
| Sequence 974 | AA281412 | |
| Sequence 975 | AA368906 | |
| Sequence 976 | AA101820 | |
| Sequence 977 | AA302450 | |
| Sequence 978 | N49477 | |
| Sequence 979 | AA399022 | |
| Sequence 980 | Al453471 | |
| Sequence 981 | AA699666 | |
| Sequence 982 | AA372894 | |
| Sequence 983 | Al217001 | |
| Sequence 984 | AA328393 | |
| Sequence 985 | Al249705 | |
| Sequence 986 | Al090943 | |
| Sequence 987 | AA126836 | |
| Sequence 988 | Al536912 | |
| Sequence 989 | AA935134 | |
| Sequence 990 | AA994818 | |
| Sequence 991 | Al342714 | |
| Sequence 992 | AA916068 | |
| Sequence 993 | AA375384 | |
| Sequence 994 | AA310694 | |
| Sequence 995 | AA037823 | |
| Sequence 996 | AA190559 | |
| Sequence 997 | AA332853 | |
| Sequence 998 | AA827899 | |
| Sequence 999 | AA065319 | |
| Sequence 1000 | AA573238 | |
| Sequence 1001 | AA573238 | |
| Sequence 1002 | N99772 | |
| Sequence 1003 | AA192253 | |
| Sequence 1004 | Al017852 | |
| Sequence 1005 | AA203140 | |
| Sequence 1006 | AA345827 | |
| Sequence 1007 | Al129468 | |
| Sequence 1008 | AA151675 | |
| Sequence 1009 | AA157619 | |
| Sequence 1010 | Al204088 | |
| Sequence 1011 | AA232691 | |
| Sequence 1012 | AA232691 | |
| Sequence 1013 | Al267454 | |
| Sequence 1014 | AA464646 | |
| Sequence 1015 | AA496961 | |
| Sequence 1016 | | AA496961 |
| Sequence 1017 | AA779747 | |
| Sequence 1018 | Al433157 | |
| Sequence 1019 | AA464646 | |
| Sequence 1020 | Al311440 | |
| Sequence 1021 | Al127835 | |
| Sequence 1022 | AA279553 | |
| Sequence 1023 | AA227876 | |
| Sequence 1024 | AA455007 | |
| Sequence 1025 | AA169457 | |
| Sequence 1026 | AA169457 | |

19/56

| | |
|---|---|
| Sequence 1027 | AA766864 |
| Sequence 1028 | AA026454 |
| Sequence 1029 | AA026454 |
| Sequence 1030 | Al074333 |
| Sequence 1031 | Al074333 |
| Sequence 1032 | AA448332 |
| Sequence 1033 | F27264 |
| Sequence 1034 | AA188481 |
| Sequence 1035 | Al738508 |
| Sequence 1036 | Al738508 |
| Sequence 1037 | AA928420 |
| Sequence 1038 | Al742056 |
| Sequence 1039 | AA489412 |
| Sequence 1040 | Al066421 |
| Sequence 1041 | AA368063 |
| Sequence 1042 | Z45013 |
| Sequence 1043 | AA490731 |
| Sequence 1044 | AA316071 |
| Sequence 1045 | AA872059 |
| Sequence 1046 | AA083836 |
| Sequence 1047 | AA199821 |
| Sequence 1048 | AA187824 |
| Sequence 1049 | AA913590 |
| Sequence 1050 | AA913590 |
| Sequence 1051 | Al074572 |
| Sequence 1052 | AA101077 |
| Sequence 1053 | AA668553 |
| Sequence 1054 | AA165471 |
| Sequence 1055 | AA286751 |
| Sequence 1056 | Al440495 |
| Sequence 1057 | AA526893 |
| Sequence 1058 | Al278160 |
| Sequence 1059 | Al242593 |
| Sequence 1060 | AA622574 |
| Sequence 1061 | Al591008 |
| Sequence 1062 | AA365398 |
| Sequence 1063 | AA173885 |
| Sequence 1064 | T08682 |
| Sequence 1065 | AA074869 |
| Sequence 1066 | AA357299 |
| Sequence 1067 | AA203140 |
| Sequence 1068 | AA190559 |
| Sequence 1069 | Al168018 |
| Sequence 1070 | AA451894 |
| Sequence 1071 | AA024859 |
| Sequence 1072 | AA412184 |
| Sequence 1073 | AA207216 |
| Sequence 1074 | AA463446 |
| Sequence 1075 | AA135572 |
| Sequence 1076 | Al364978 |
| Sequence 1077 | Al281972 |
| Sequence 1078 | AA418408 |
| Sequence 1079 | AA418408 |
| Sequence 1080 | AA074098 |
| Sequence 1081 | AA029567 |
| Sequence 1082 | AA747547 |
| Sequence 1083 | Al753431 |

20/56

| | |
|---|---|
| Sequence 1084 | AA232691 |
| Sequence 1085 | Al079871 |
| Sequence 1086 | Al300541 |
| Sequence 1087 | AA417006 |
| Sequence 1088 | AA490731 |
| Sequence 1089 | Z45013 |
| Sequence 1090 | Al066421 |
| Sequence 1091 | AA621714 |
| Sequence 1092 | AA813524 |
| Sequence 1093 | AA515261 |
| Sequence 1094 | AA040300 |
| Sequence 1095 | AA040300 |
| Sequence 1096 | Al241763 |
| Sequence 1097 | AA722512 |
| Sequence 1098 | Al034147 |
| Sequence 1099 | Al703111 |
| Sequence 1100 | Al079871 |
| Sequence 1101 | Al079871 |
| Sequence 1102 | AA485781 |
| Sequence 1103 | AA216667 |
| Sequence 1104 | AA216667 |
| Sequence 1105 | Al267282 |
| Sequence 1106 | AA485488 |
| Sequence 1107 | AA295804 |
| Sequence 1108 | AA601147 |
| Sequence 1109 | H06792 |
| Sequence 1110 | AA283707 |
| Sequence 1111 | AA101077 |
| Sequence 1112 | Al076532 |
| Sequence 1113 | Al076532 |
| Sequence 1114 | AA609534 |
| Sequence 1115 | AA232691 |
| Sequence 1116 | R60403 |
| Sequence 1117 | AA928676 |
| Sequence 1118 | AA807960 |
| Sequence 1119 | AA573742 |
| Sequence 1120 | AA463547 |
| Sequence 1121 | Al086042 |
| Sequence 1122 | AI017852 |
| Sequence 1123 | Al267185 |
| Sequence 1124 | AA167794 |
| Sequence 1125 | Al253335 |
| Sequence 1126 | AA005232 |
| Sequence 1127 | AA581144 |
| Sequence 1128 | Al204127 |
| Sequence 1129 | AA420989 |
| Sequence 1130 | AA877877 |
| Sequence 1131 | AA364275 |
| Sequence 1132 | AA455007 |
| Sequence 1133 | AA091539 |
| Sequence 1134 | AA088373 |
| Sequence 1135 | AA088373 |
| Sequence 1136 | AA595217 |
| Sequence 1137 | AA642691 |
| Sequence 1138 | Al417520 |
| Sequence 1139 | AA133375 |
| Sequence 1140 | Al472536 |

21/56

| | |
|---|---|
| Sequence 1141 | AA772548 |
| Sequence 1142 | AA772548 |
| Sequence 1143 | AA161488 |
| Sequence 1144 | AA609253 |
| Sequence 1145 | AA412184 |
| Sequence 1146 | AA679015 |
| Sequence 1147 | AA280235 |
| Sequence 1148 | Al080011 |
| Sequence 1149 | Al091013 |
| Sequence 1150 | AA905362 |
| Sequence 1151 | AA521069 |
| Sequence 1152 | AA226674 |
| Sequence 1153 | AA226674 |
| Sequence 1154 | AA037506 |
| Sequence 1155 | AA522790 |
| Sequence 1156 | AA375384 |
| Sequence 1157 | AA788933 |
| Sequence 1158 | Al418922 |
| Sequence 1159 | AA420989 |
| Sequence 1160 | AA323097 |
| Sequence 1161 | Al432949 |
| Sequence 1162 | Al432949 |
| Sequence 1163 | AA226914 |
| Sequence 1164 | AA442097 |
| Sequence 1165 | AA316566 |
| Sequence 1166 | H98646 |
| Sequence 1167 | Al281417 |
| Sequence 1168 | AA283707 |
| Sequence 1169 | AA505080 |
| Sequence 1170 | AA505080 |
| Sequence 1171 | AA789020 |
| Sequence 1172 | AA789020 |
| Sequence 1173 | N30234 |
| Sequence 1174 | N98658 |
| Sequence 1175 | AA323560 |
| Sequence 1176 | AA719473 |
| Sequence 1177 | AA446943 |
| Sequence 1178 | AA419395 |
| Sequence 1179 | AA419395 |
| Sequence 1180 | Al718403 |
| Sequence 1181 | AA115025 |
| Sequence 1182 | AA789013 |
| Sequence 1183 | AA830249 |
| Sequence 1184 | AA830249 |
| Sequence 1185 | AA282848 |
| Sequence 1186 | AA136215 |
| Sequence 1187 | AA336515 |
| Sequence 1188 | AA854982 |
| Sequence 1189 | AA533727 |
| Sequence 1190 | AA121207 |
| Sequence 1191 | AA424541 |
| Sequence 1192 | AA040300 |
| Sequence 1193 | AA923750 |
| Sequence 1194 | AA173885 |
| Sequence 1195 | AA352308 |
| Sequence 1196 | AA233105 |
| Sequence 1197 | H71437 |

22/56

| | | |
|---|---|---|
| Sequence 1198 | Al076532 | |
| Sequence 1199 | AA650333 | |
| Sequence 1200 | AA702428 | |
| Sequence 1201 | AA531613 | |
| Sequence 1202 | AA255584 | |
| Sequence 1203 | AA255584 | |
| Sequence 1204 | Al141699 | |
| Sequence 1205 | AA481432 | |
| Sequence 1206 | AA305599 | |
| Sequence 1207 | H10703 | |
| Sequence 1208 | AA121879 | |
| Sequence 1209 | Al249797 | |
| Sequence 1210 | AA482853 | |
| Sequence 1211 | Al141567 | |
| Sequence 1212 | H01413 | |
| Sequence 1213 | AA148508 | |
| Sequence 1214 | AA729727 | |
| Sequence 1215 | AA789158 | |
| Sequence 1216 | AA679848 | |
| Sequence 1217 | AA679848 | |
| Sequence 1218 | Al221967 | |
| Sequence 1219 | AA838613 | |
| Sequence 1220 | Al223887 | |
| Sequence 1221 | W57570 | |
| Sequence 1222 | H23090 | |
| Sequence 1223 | Al203342 | |
| Sequence 1224 | AA056334 | |
| Sequence 1225 | Al088115 | |
| Sequence 1226 | AA373243 | |
| Sequence 1227 | Al359989 | |
| Sequence 1228 | A | A247180 |
| Sequence 1229 | AA838613 | |
| Sequence 1230 | AA136215 | |
| Sequence 1231 | AA876375 | |
| Sequence 1232 | AA876375 | |
| Sequence 1233 | AA905495 | |
| Sequence 1234 | Al344928 | |
| Sequence 1235 | AA769315 | |
| Sequence 1236 | F37855 | |
| Sequence 1237 | Al093165 | |
| Sequence 1238 | AA478787 | |
| Sequence 1239 | AA088344 | |
| Sequence 1240 | AA766044 | |
| Sequence 1241 | Al339481 | |
| Sequence 1242 | AA829473 | |
| Sequence 1243 | 020248 | |
| Sequence 1244 | AA412015 | |
| Sequence 1245 | AA420989 | |
| Sequence 1246 | AA259065 | |
| Sequence 1247 | Al345847 | |
| Sequence 1248 | AA235453 | |
| Sequence 1249 | AA486335 | |
| Sequence 1250 | Al129482 | |
| Sequence 1251 | Al690374 | |
| Sequence 1252 | Al034481 | |
| Sequence 1253 | Al312562 | |
| Sequence 1254 | Al312562 | |

23/56

| | |
|---|---|
| Sequence 1255 | Al092921 |
| Sequence 1256 | AA570668 |
| Sequence 1257 | AA489032 |
| Sequence 1258 | AA047729 |
| Sequence 1259 | AA662918 |
| Sequence 1260 | Al078151 |
| Sequence 1261 | AA233672 |
| Sequence 1262 | AA157424 |
| Sequence 1263 | Al078151 |
| Sequence 1264 | AA043258 |
| Sequence 1265 | AA126623 |
| Sequence 1266 | AA062936 |
| Sequence 1267 | AA062936 |
| Sequence 1268 | AA147951 |
| Sequence 1269 | AA148100 |
| Sequence 1270 | AA281556 |
| Sequence 1271 | AA482303 |
| Sequence 1272 | Al276690 |
| Sequence 1273 | AA303709 |
| Sequence 1274 | Al004675 |
| Sequence 1275 | AA039354 |
| Sequence 1276 | AA563987 |
| Sequence 1277 | AA488634 |
| Sequence 1278 | AA399628 |
| Sequence 1279 | Al267185 |
| Sequence 1280 | AA226674 |
| Sequence 1281 | AA469121 |
| Sequence 1282 | AA652436 |
| Sequence 1283 | Al671174 |
| Sequence 1284 | AA446040 |
| Sequence 1285 | Al309007 |
| Sequence 1286 | Al201573 |
| Sequence 1287 | 016203 |
| Sequence 1288 | AA035003 |
| Sequence 1289 | AA838431 |
| Sequence 1290 | AA838431 |
| Sequence 1291 | AA007569 |
| Sequence 1292 | AA853131 |
| Sequence 1293 | AA190559 |
| Sequence 1294 | AA155915 |
| Sequence 1295 | Al023372 |
| Sequence 1296 | Al276690 |
| Sequence 1297 | Al199515 |
| Sequence 1298 | Al199515 |
| Sequence 1299 | Al025517 |
| Sequence 1300 | AA127565 |
| Sequence 1301 | AA526498 |
| Sequence 1302 | AA114138 |
| Sequence 1303 | AA765228 |
| Sequence 1304 | AA765228 |
| Sequence 1305 | Al090077 |
| Sequence 1306 | AA044748 |
| Sequence 1307 | AA044748 |
| Sequence 1308 | Al267502 |
| Sequence 1309 | AA344583 |
| Sequence 1310 | AA029043 |
| Sequence 1311 | H52011 |

24/56

| | |
|---|---|
| Sequence 1312 | AA700350 |
| Sequence 1313 | AA483053 |
| Sequence 1314 | AA679015 |
| Sequence 1315 | M430369 |
| Sequence 1316 | Al309007 |
| Sequence 1317 | AA005232 |
| Sequence 1318 | AA102563 |
| Sequence 1319 | H98646 |
| Sequence 1320 | AA548889 |
| Sequence 1321 | AI267185 |
| Sequence 1322 | W19131 |
| Sequence 1323 | AA847560 |
| Sequence 1324 | AA279747 |
| Sequence 1325 | AA025721 |
| Sequence 1326 | AA679015 |
| Sequence 1327 | AA428184 |
| Sequence 1328 | Al273856 |
| Sequence 1329 | AA514959 |
| Sequence 1330 | AA452335 |
| Sequence 1331 | AA165463 |
| Sequence 1332 | AA514542 |
| Sequence 1333 | F32233 |
| Sequence 1334 | AA400840 |
| Sequence 1335 | AA385602 |
| Sequence 1336 | Al075412 |
| Sequence 1337 | AA772548 |
| Sequence 1338 | AA772548 |
| Sequence 1339 | AA679015 |
| Sequence 1340 | Al267282 |
| Sequence 1341 | AA075148 |
| Sequence 1342 | Al624304 |
| Sequence 1343 | Al436387 |
| Sequence 1344 | AA166622 |
| Sequence 1345 | AA916110 |
| Sequence 1346 | AA047065 |
| Sequence 1347 | AA399022 |
| Sequence 1348 | AA136215 |
| Sequence 1349 | AA039354 |
| Sequence 1350 | AA634397 |
| Sequence 1351 | AA788933 |
| Sequence 1352 | AA039354 |
| Sequence 1353 | AA323602 |
| Sequence 1354 | Al332433 |
| Sequence 1355 | AA446215 |
| Sequence 1356 | AA082498 |
| Sequence 1357 | AA464646 |
| Sequence 1358 | AA465164 |
| Sequence 1359 | Al754431 |
| Sequence 1360 | Al754431 |
| Sequence 1361 | H01401 |
| Sequence 1362 | H01401 |
| Sequence 1363 | AF001541 |
| Sequence 1364 | AA047729 |
| Sequence 1365 | AA399022 |
| Sequence 1366 | Al127825 |
| Sequence 1367 | Al089220 |
| Sequence 1368 | AA770397 |

25/56

| | |
|---|---|
| Sequence 1369 | AA305494 |
| Sequence 1370 | AA083004 |
| Sequence 1371 | AA151600 |
| Sequence 1372 | AA233641 |
| Sequence 1373 | AA226674 |
| Sequence 1374 | AA453784 |
| Sequence 1375 | AA853539 |
| Sequence 1376 | AA449456 |
| Sequence 1377 | Al625806 |
| Sequence 1378 | Al348661 |
| Sequence 1379 | AA608668 |
| Sequence 1380 | AA303709 |
| Sequence 1381 | AA612685 |
| Sequence 1382 | AA113235 |
| Sequence 1383 | AA576065 |
| Sequence 1384 | Al033304 |
| Sequence 1385 | Al090509 |
| Sequence 1386 | D81636 |
| Sequence 1387 | AA232447 |
| Sequence 1388 | AA608790 |
| Sequence 1389 | AA307513 |
| Sequence 1390 | Al267185 |
| Sequence 1391 | Al491897 |
| Sequence 1392 | AA983883 |
| Sequence 1393 | AA171834 |
| Sequence 1394 | Al262064 |
| Sequence 1395 | Al473830 |
| Sequence 1396 | AA126418 |
| Sequence 1397 | AA487845 |
| Sequence 1398 | AA700903 |
| Sequence 1399 | AA977584 |
| Sequence 1400 | AA091539 |
| Sequence 1401 | AA580412 |
| Sequence 1402 | AA580412 |
| Sequence 1403 | AA182766 |
| Sequence 1404 | AA053874 |
| Sequence 1405 | Al090863 |
| Sequence 1406 | Al271883 |
| Sequence 1407 | AA101862 |
| Sequence 1408 | N24636 |
| Sequence 1409 | AA112046 |
| Sequence 1410 | Al421699 |
| Sequence 1411 | Al738508 |
| Sequence 1412 | Al738508 |
| Sequence 1413 | AA464646 |
| Sequence 1414 | AA234411 |
| Sequence 1415 | AA595343 |
| Sequence 1416 | AA314225 |
| Sequence 1417 | AA706004 |
| Sequence 1418 | AA704151 |
| Sequence 1419 | Al267185 |
| Sequence 1420 | AA075148 |
| Sequence 1421 | AA026719 |
| Sequence 1422 | AA420758 |
| Sequence 1423 | Al362945 |
| Sequence 1424 | AA936566 |
| Sequence 1425 | AA063512 |

26/56

| | |
|---|---|
| Sequence 1426 | AA112408 |
| Sequence 1427 | Al478691 |
| Sequence 1428 | AA694186 |
| Sequence 1429 | AA026719 |
| Sequence 1430 | AA541386 |
| Sequence 1431 | T71404 |
| Sequence 1432 | T71404 |
| Sequence 1433 | AA228025 |
| Sequence 1434 | AA228025 |
| Sequence 1435 | AA493211 |
| Sequence 1436 | AA228895 |
| Sequence 1437 | AA398144 |
| Sequence 1438 | Al309007 |
| Sequence 1439 | Al127815 |
| Sequence 1440 | Al127815 |
| Sequence 1441 | AF001541 |
| Sequence 1442 | AA047729 |
| Sequence 1443 | Al262125 |
| Sequence 1444 | AA459456 |
| Sequence 1445 | AA459456 |
| Sequence 1446 | AA913590 |
| Sequence 1447 | AA913590 |
| Sequence 1448 | Al241000 |
| Sequence 1449 | AA769847 |
| Sequence 1450 | AA522903 |
| Sequence 1451 | AA725363 |
| Sequence 1452 | Al093315 |
| Sequence 1453 | Al188929 |
| Sequence 1454 | H17020 |
| Sequence 1455 | AA573742 |
| Sequence 1456 | AA307513 |
| Sequence 1457 | Al144526 |
| Sequence 1458 | Al167693 |
| Sequence 1459 | AA506302 |
| Sequence 1460 | W78198 |
| Sequence 1461 | AA258725 |
| Sequence 1462 | Al887267 |
| Sequence 1463 | AA233109 |
| Sequence 1464 | R22457 |
| Sequence 1465 | Al344311 |
| Sequence 1466 | Al344311 |
| Sequence 1467 | AA128261 |
| Sequence 1468 | Al311789 |
| Sequence 1469 | AA612685 |
| Sequence 1470 | AA316516 |
| Sequence 1471 | AA316516 |
| Sequence 1472 | AA830249 |
| Sequence 1473 | AA424829 |
| Sequence 1474 | N46402 |
| Sequence 1475 | AA946876 |
| Sequence 1476 | AA411763 |
| Sequence 1477 | AA057400 |
| Sequence 1478 | AA609169 |
| Sequence 1479 | Al310129 |
| Sequence 1480 | Al679214 |
| Sequence 1481 | AA831877 |
| Sequence 1482 | AA653693 |

27/56

| | |
|---|---|
| Sequence 1483 | N98658 |
| Sequence 1484 | AA580144 |
| Sequence 1485 | AA219116 |
| Sequence 1486 | D81636 |
| Sequence 1487 | R35844 |
| Sequence 1488 | Al493233 |
| Sequence 1489 | AA714531 |
| Sequence 1490 | AA531487 |
| Sequence 1491 | AA765031 |
| Sequence 1492 | AA765031 |
| Sequence 1493 | AA191088 |
| Sequence 1494 | AA193645 |
| Sequence 1495 | AA376374 |
| Sequence 1496 | AA864690 |
| Sequence 1497 | AA453784 |
| Sequence 1498 | AA453784 |
| Sequence 1499 | W44970 |
| Sequence 1500 | W44970 |
| Sequence 1501 | H52011 |
| Sequence 1502 | Al678238 |
| Sequence 1503 | Al360579 |
| Sequence 1504 | H52011 |
| Sequence 1505 | H52011 |
| Sequence 1506 | AA286693 |
| Sequence 1507 | AA233689 |
| Sequence 1508 | AA453784 |
| Sequence 1509 | AA453784 |
| Sequence 1510 | AA788933 |
| Sequence 1511 | AA349417 |
| Sequence 1512 | AA101077 |
| Sequence 1513 | Al41537 |
| Sequence 1514 | AA075451 |
| Sequence 1515 | AA258725 |
| Sequence 1516 | Al309007 |
| Sequence 1517 | AA526060 |
| Sequence 1518 | AA256330 |
| Sequence 1519 | AA156569 |
| Sequence 1520 | AA130228 |
| Sequence 1521 | D61455 |
| Sequence 1522 | AA864690 |
| Sequence 1523 | AA349978 |
| Sequence 1524 | AA446888 |
| Sequence 1525 | Al254200 |
| Sequence 1526 | AA151282 |
| Sequence 1527 | AA614671 |
| Sequence 1528 | Al494235 |
| Sequence 1529 | AA410835 |
| Sequence 1530 | AA090891 |
| Sequence 1531 | Al392999 |
| Sequence 1532 | AA604210 |
| Sequence 1533 | AA974657 |
| Sequence 1534 | AA765228 |
| Sequence 1535 | AA070635 |
| Sequence 1536 | AA974049 |
| Sequence 1537 | AA644001 |
| Sequence 1538 | AA037314 |
| Sequence 1539 | AA037314 |

28/56

| | |
|---|---|
| Sequence 1540 | AA214710 |
| Sequence 1541 | AA196135 |
| Sequence 1542 | AA227890 |
| Sequence 1543 | AA451869 |
| Sequence 1544 | AA769847 |
| Sequence 1545 | T25387 |
| Sequence 1546 | Al342714 |
| Sequence 1547 | Al342714 |
| Sequence 1548 | AA315103 |
| Sequence 1549 | AA453217 |
| Sequence 1550 | AA349978 |
| Sequence 1551 | Al652058 |
| Sequence 1552 | Al052317 |
| Sequence 1553 | AA082498 |
| Sequence 1554 | AA662648 |
| Sequence 1555 | AA225164 |
| Sequence 1556 | AA092923 |
| Sequence 1557 | Al193577 |
| Sequence 1558 | N78081 |
| Sequence 1559 | AA243394 |
| Sequence 1560 | AA100384 |
| Sequence 1561 | Al609252 |
| Sequence 1562 | Al609252 |
| Sequence 1563 | AA075507 |
| Sequence 1564 | AA961734 |
| Sequence 1565 | AA148011 |
| Sequence 1566 | Al077325 |
| Sequence 1567 | Al077325 |
| Sequence 1568 | AA523877 |
| Sequence 1569 | AA039354 |
| Sequence 1570 | AA101077 |
| Sequence 1571 | AA083471 |
| Sequence 1572 | AA723444 |
| Sequence 1573 | AA788933 |
| Sequence 1574 | AA552818 |
| Sequence 1575 | AA088200 |
| Sequence 1576 | AA021648 |
| Sequence 1577 | AA333713 |
| Sequence 1578 | AA702428 |
| Sequence 1579 | Al342714 |
| Sequence 1580 | Z44537 |
| Sequence 1581 | AA424680 |
| Sequence 1582 | AA252440 |
| Sequence 1583 | Al004348 |
| Sequence 1584 | AA738116 |
| Sequence 1585 | AA454514 |
| Sequence 1586 | AA082795 |
| Sequence 1587 | AA242997 |
| Sequence 1588 | AA216022 |
| Sequence 1589 | AA157054 |
| Sequence 1590 | Al278872 |
| Sequence 1591 | AA258725 |
| Sequence 1592 | AA258725 |
| Sequence 1593 | AA573742 |
| Sequence 1594 | AA165638 |
| Sequence 1595 | Al435429 |
| Sequence 1596 | AA639701 |

29/56

| | | |
|---|---|---|
| Sequence 1597 | AA788933 | |
| Sequence 1598 | AA707546 | |
| Sequence 1599 | AA243767 | |
| Sequence 1600 | AA243767 | |
| Sequence 1601 | AA300170 | |
| Sequence 1602 | AA678586 | |
| Sequence 1603 | Al435276 | |
| Sequence 1604 | AA157680 | |
| Sequence 1605 | AA131338 | |
| Sequence 1606 | AA704512 | |
| Sequence 1607 | Al630387 | |
| Sequence 1608 | AA255567 | |
| Sequence 1609 | AA255567 | |
| Sequence 1610 | N46402 | |
| Sequence 1611 | AA531504 | |
| Sequence 1612 | Al351167 | |
| Sequence 1613 | AA523877 | |
| Sequence 1614 | AA181734 | |
| Sequence 1615 | AA608623 | |
| Sequence 1616 | D81635 | |
| Sequence 1617 | Al148825 | |
| Sequence 1618 | AA122401 | |
| Sequence 1619 | AA186758 | |
| Sequence 1620 | AA074086 | |
| Sequence 1621 | AA216132 | |
| Sequence 1622 | AA131231 | |
| Sequence 1623 | AA769937 | |
| Sequence 1624 | AA376374 | |
| Sequence 1625 | AA412184 | |
| Sequence 1626 | AA788933 | |
| Sequence 1627 | AA258116 | |
| Sequence 1628 | R54092 | |
| Sequence 1629 | AA306488 | |
| Sequence 1630 | H52011 | |
| Sequence 1631 | A | A947616 |
| Sequence 1632 | AA317903 | |
| Sequence 1633 | AA079835 | |
| Sequence 1634 | Al131245 | |
| Sequence 1635 | AA136215 | |
| Sequence 1636 | AA113359 | |
| Sequence 1637 | AA375933 | |
| Sequence 1638 | AA469304 | |
| Sequence 1639 | AA521069 | |
| Sequence 1640 | AA111853 | |
| Sequence 1641 | D14533 | |
| Sequence 1642 | X13111 | |
| Sequence 1643 | K00799 | |
| Sequence 1644 | D10522 | |
| Sequence 1645 | AB007952 | |
| Sequence 1646 | X04408 | |
| Sequence 1647 | X04408 | |
| Sequence 1648 | AB002368 | |
| Sequence 1649 | X73608 | |
| Sequence 1650 | X73608 | |
| Sequence 1651 | M55409 | |
| Sequence 1652 | D83327 | |
| Sequence 1653 | M10905 | |

30/56

| | |
|---|---|
| Sequence 1654 | M30257 |
| Sequence 1655 | AF086204 |
| Sequence 1656 | AF007216 |
| Sequence 1657 | M77830 |
| Sequence 1658 | AF052113 |
| Sequence 1659 | D38583 |
| Sequence 1660 | L13385 |
| Sequence 1661 | L13385 |
| Sequence 1662 | M17885 |
| Sequence 1663 | AF016535 |
| Sequence 1664 | AF016535 |
| Sequence 1665 | AF068302 |
| Sequence 1666 | X04098 |
| Sequence 1667 | L10612 |
| Sequence 1668 | X07549 |
| Sequence 1669 | M31470 |
| Sequence 1670 | M95724 |
| Sequence 1671 | AJ010841 |
| Sequence 1672 | Y07968 |
| Sequence 1673 | AF035286 |
| Sequence 1674 | AB024704 |
| Sequence 1675 | D00726 |
| Sequence 1676 | J02943 |
| Sequence 1677 | J02943 |
| Sequence 1678 | X87949 |
| Sequence 1679 | M10941 |
| Sequence 1680 | US1586 |
| Sequence 1681 | AF004429 |
| Sequence 1682 | E02628 |
| Sequence 1683 | D87432 |
| Sequence 1684 | X07897 |
| Sequence 1685 | U39318 |
| Sequence 1686 | J03202 |
| Sequence 1687 | J03202 |
| Sequence 1688 | U34074 |
| Sequence 1689 | U42404 |
| Sequence 1690 | AF121860 |
| Sequence 1691 | D50918 |
| Sequence 1692 | D50918 |
| Sequence 1693 | X04098 |
| Sequence 1694 | X04098 |
| Sequence 1695 | U14968 |
| Sequence 1696 | U42404 |
| Sequence 1697 | AB014577 |
| Sequence 1698 | U42404 |
| Sequence 1699 | U42404 |
| Sequence 1700 | X64707 |
| Sequence 1701 | AF127918 |
| Sequence 1702 | AF127918 |
| Sequence 1703 | E01650 |
| Sequence 1704 | M64241 |
| Sequence 1705 | M64241 |
| Sequence 1706 | E02628 |
| Sequence 1707 | X04098 |
| Sequence 1708 | AL050161 |
| Sequence 1709 | U42404 |
| Sequence 1710 | X03963 |

31/56

| | |
|---|---|
| Sequence 1711 | D86322 |
| Sequence 1712 | U42404 |
| Sequence 1713 | U42404 |
| Sequence 1714 | U51586 |
| Sequence 1715 | M34788 |
| Sequence 1716 | M34788 |
| Sequence 1717 | M58297 |
| Sequence 1718 | AF035286 |
| Sequence 1719 | M10905 |
| Sequence 1720 | X04098 |
| Sequence 1721 | U05598 |
| Sequence 1722 | AF083322 |
| Sequence 1723 | X04098 |
| Sequence 1724 | X77956 |
| Sequence 1725 | AJ000147 |
| Sequence 1726 | M58297 |
| Sequence 1727 | E02628 |
| Sequence 1728 | AB002533 |
| Sequence 1729 | U42404 |
| Sequence 1730 | X03963 |
| Sequence 1731 | L11066 |
| Sequence 1732 | D17268 |
| Sequence 1733 | AL050179 |
| Sequence 1734 | AB007896 |
| Sequence 1735 | U07919 |
| Sequence 1736 | K00799 |
| Sequence 1737 | U61837 |
| Sequence 1738 | AF083322 |
| Sequence 1739 | U42404 |
| Sequence 1740 | U42404 |
| Sequence 1741 | X85373 |
| Sequence 1742 | E02326 |
| Sequence 1743 | Z11695 |
| Sequence 1744 | U07919 |
| Sequence 1745 | AB014511 |
| Sequence 1746 | AB014511 |
| Sequence 1747 | X79535 |
| Sequence 1748 | X79535 |
| Sequence 1749 | U42404 |
| Sequence 1750 | U42404 |
| Sequence 1751 | M11353 |
| Sequence 1752 | AF070561 |
| Sequence 1753 | AF070561 |
| Sequence 1754 | AL021683 |
| Sequence 1755 | M27508 |
| Sequence 1756 | M22590 |
| Sequence 1757 | AF070648 |
| Sequence 1758 | AJ010841 |
| Sequence 1759 | M23254 |
| Sequence 1760 | D80005 |
| Sequence 1761 | M94345 |
| Sequence 1762 | AF070524 |
| Sequence 1763 | L42542 |
| Sequence 1764 | U42404 |
| Sequence 1765 | U42404 |
| Sequence 1766 | AF070524 |
| Sequence 1767 | AF070524 |

32/56

| | |
|---|---|
| Sequence 1768 | AF028832 |
| Sequence 1769 | U42457 |
| Sequence 1770 | K00799 |
| Sequence 1771 | M96982 |
| Sequence 1772 | X85373 |
| Sequence 1773 | L33930 |
| Sequence 1774 | M22590 |
| Sequence 1775 | M22590 |
| Sequence 1776 | X84407 |
| Sequence 1777 | U42404 |
| Sequence 1778 | X79535 |
| Sequence 1779 | AB002533 |
| Sequence 1780 | X84407 |
| Sequence 1781 | X13238 |
| Sequence 1782 | AF086336 |
| Sequence 1783 | AB021654 |
| Sequence 1784 | U07919 |
| Sequence 1785 | AJ223812 |
| Sequence 1786 | U42404 |
| Sequence 1787 | X07897 |
| Sequence 1788 | U42404 |
| Sequence 1789 | AB019564 |
| Sequence 1790 | M27508 |
| Sequence 1791 | AF070561 |
| Sequence 1792 | AF070561 |
| Sequence 1793 | D87930 |
| Sequence 1794 | EQ1650 |
| Sequence 1795 | U42457 |
| Sequence 1796 | D25542 |
| Sequence 1797 | AB004903 |
| Sequence 1798 | AB004903 |
| Sequence 1799 | M37583 |
| Sequence 1800 | M69181 |
| Sequence 1801 | M69181 |
| Sequence 1802 | M69181 |
| Sequence 1803 | S80562 |
| Sequence 1804 | U84573 |
| Sequence 1805 | AF028832 |
| Sequence 1806 | M33308 |
| Sequence 1807 | AL035081 |
| Sequence 1808 | AF070664 |
| Sequence 1809 | AJ011007 |
| Sequence 1810 | AB018346 |
| Sequence 1811 | D42054 |
| Sequence 1812 | M58510 |
| Sequence 1813 | K00799 |
| Sequence 1814 | E02326 |
| Sequence 1815 | M16279 |
| Sequence 1816 | U42404 |
| Sequence 1817 | AL035081 |
| Sequence 1818 | AB007862 |
| Sequence 1819 | E01650 |
| Sequence 1820 | E01650 |
| Sequence 1821 | M23254 |
| Sequence 1822 | M23254 |
| Sequence 1823 | AB003102 |
| Sequence 1824 | L25610 |

33/56

| | |
|---|---|
| Sequence 1825 | M16279 |
| Sequence 1826 | J02943 |
| Sequence 1827 | AL049974 |
| Sequence 1828 | AF098462 |
| Sequence 1829 | M15502 |
| Sequence 1830 | E01650 |
| Sequence 1831 | AF084260 |
| Sequence 1832 | AF120268 |
| Sequence 1833 | AJ010841 |
| Sequence 1834 | AJ010841 |
| Sequence 1835 | U42404 |
| Sequence 1836 | U90545 |
| Sequence 1837 | X07897 |
| Sequence 1838 | AB007862 |
| Sequence 1839 | AF011468 |
| Sequence 1840 | L38933 |
| Sequence 1841 | U07343 |
| Sequence 1842 | M69066 |
| Sequence 1843 | L05093 |
| Sequence 1844 | X04098 |
| Sequence 1845 | AF143324 |
| Sequence 1846 | D12676 |
| Sequence 1847 | Y11312 |
| Sequence 1848 | U14970 |
| Sequence 1849 | X81198 |
| Sequence 1850 | AF021819 |
| Sequence 1851 | J03576 |
| Sequence 1852 | M10905 |
| Sequence 1853 | U42404 |
| Sequence 1854 | U42404 |
| Sequence 1855 | U42404 |
| Sequence 1856 | AB018346 |
| Sequence 1857 | AB012083 |
| Sequence 1858 | X02761 |
| Sequence 1859 | X81198 |
| Sequence 1860 | U64898 |
| Sequence 1861 | 064015 |
| Sequence 1862 | Y09565 |
| Sequence 1863 | AF007145 |
| Sequence 1864 | E01650 |
| Sequence 1865 | U57847 |
| Sequence 1866 | M69066 |
| Sequence 1867 | U41850 |
| Sequence 1868 | AJ006834 |
| Sequence 1869 | 012485 |
| Sequence 1870 | AJ010841 |
| Sequence 1871 | AB019568 |
| Sequence 1872 | AB014560 |
| Sequence 1873 | X97064 |
| Sequence 1874 | U42404 |
| Sequence 1875 | J00127 |
| Sequence 1876 | AF086002 |
| Sequence 1877 | U14969 |
| Sequence 1878 | U42404 |
| Sequence 1879 | AF119386 |
| Sequence 1880 | AB002533 |
| Sequence 1881 | J03634 |

34/56

| | |
|---|---|
| Sequence 1882 | M74775 |
| Sequence 1883 | D26124 |
| Sequence 1884 | J03460 |
| Sequence 1885 | AF151885 |
| Sequence 1886 | D49737 |
| Sequence 1887 | U42404 |
| Sequence 1888 | AF116827 |
| Sequence 1889 | M14083 |
| Sequence 1890 | Z47087 |
| Sequence 1891 | M23254 |
| Sequence 1892 | AF054990 |
| Sequence 1893 | M22324 |
| Sequence 1894 | AF077045 |
| Sequence 1895 | U42457 |
| Sequence 1896 | AB020660 |
| Sequence 1897 | D80005 |
| Sequence 1898 | X64707 |
| Sequence 1899 | AL021683 |
| Sequence 1900 | AF151885 |
| Sequence 1901 | D32000 |
| Sequence 1902 | S82470 |
| Sequence 1903 | AL049381 |
| Sequence 1904 | X81198 |
| Sequence 1905 | D50918 |
| Sequence 1906 | D50918 |
| Sequence 1907 | AF070561 |
| Sequence 1908 | S78569 |
| Sequence 1909 | U02390 |
| Sequence 1910 | U34074 |
| Sequence 1911 | U33760 |
| Sequence 1912 | E03413 |
| Sequence 1913 | J03202 |
| Sequence 1914 | M55268 |
| Sequence 1915 | J03537 |
| Sequence 1916 | D21260 |
| Sequence 1917 | AF044671 |
| Sequence 1918 | AF044671 |
| Sequence 1919 | M22918 |
| Sequence 1920 | AF054990 |
| Sequence 1921 | M69181 |
| Sequence 1922 | AF006084 |
| Sequence 1923 | L16785 |
| Sequence 1924 | Y00052 |
| Sequence 1925 | U57847 |
| Sequence 1926 | M69181 |
| Sequence 1927 | X69970 |
| Sequence 1928 | X69970 |
| Sequence 1929 | AB018346 |
| Sequence 1930 | AF077045 |
| Sequence 1931 | AF077043 |
| Sequence 1932 | AF054179 |
| Sequence 1933 | AF054179 |
| Sequence 1934 | D63998 |
| Sequence 1935 | D63998 |
| Sequence 1936 | AB000220 |
| Sequence 1937 | E03157 |
| Sequence 1938 | M33308 |

35/56

| | |
|---|---|
| Sequence 1939 | L34600 |
| Sequence 1940 | AF039022 |
| Sequence 1941 | X07897 |
| Sequence 1942 | AB014548 |
| Sequence 1943 | AL021683 |
| Sequence 1944 | D86957 |
| Sequence 1945 | J04543 |
| Sequence 1946 | L13806 |
| Sequence 1947 | U12465 |
| Sequence 1948 | X87176 |
| Sequence 1949 | U42458 |
| Sequence 1950 | AB007877 |
| Sequence 1951 | AB002533 |
| Sequence 1952 | AB002533 |
| Sequence 1953 | X64707 |
| Sequence 1954 | M21551 |
| Sequence 1955 | D49737 |
| Sequence 1956 | AJ223500 |
| Sequence 1957 | D13641 |
| Sequence 1958 | L34600 |
| Sequence 1959 | AF039022 |
| Sequence 1960 | M69181 |
| Sequence 1961 | AF147319 |
| Sequence 1962 | M10119 |
| Sequence 1963 | M10119 |
| Sequence 1964 | U41515 |
| Sequence 1965 | J03202 |
| Sequence 1966 | X83218 |
| Sequence 1967 | J00127 |
| Sequence 1968 | J04543 |
| Sequence 1969 | M69181 |
| Sequence 1970 | K00799 |
| Sequence 1971 | D50918 |
| Sequence 1972 | D83198 |
| Sequence 1973 | D83198 |
| Sequence 1974 | AB011115 |
| Sequence 1975 | AB011115 |
| Sequence 1976 | AF070638 |
| Sequence 1977 | AF070638 |
| Sequence 1978 | L28010 |
| Sequence 1979 | L28010 |
| Sequence 1980 | AF031385 |
| Sequence 1981 | AF031385 |
| Sequence 1982 | AB002386 |
| Sequence 1983 | AB002386 |
| Sequence 1984 | X98248 |
| Sequence 1985 | U02032 |
| Sequence 1986 | K03195 |
| Sequence 1987 | U42404 |
| Sequence 1988 | U42404 |
| Sequence 1989 | S56985 |
| Sequence 1990 | AF077611 |
| Sequence 1991 | D49950 |
| Sequence 1992 | J03460 |
| Sequence 1993 | AF070649 |
| Sequence 1994 | M24194 |
| Sequence 1995 | X13238 |

36/56

| | |
|---|---|
| Sequence 1996 | X13238 |
| Sequence 1997 | M11058 |
| Sequence 1998 | 037991 |
| Sequence 1999 | AL050161 |
| Sequence 2000 | AJ002030 |
| Sequence 2001 | AB002533 |
| Sequence 2002 | Y00503 |
| Sequence 2003 | 087665 |
| Sequence 2004 | D87665 |
| Sequence 2005 | 029992 |
| Sequence 2006 | K00799 |
| Sequence 2007 | Y00345 |
| Sequence 2008 | D50918 |
| Sequence 2009 | D50918 |
| Sequence 2010 | L05095 |
| Sequence 2011 | AF077045 |
| Sequence 2012 | M22920 |
| Sequence 2013 | AL050218 |
| Sequence 2014 | M81757 |
| Sequence 2015 | L13210 |
| Sequence 2016 | X03963 |
| Sequence 2017 | AL050018 |
| Sequence 2018 | Y00819 |
| Sequence 2019 | M17733 |
| Sequence 2020 | U94586 |
| Sequence 2021 | U94586 |
| Sequence 2022 | J00129 |
| Sequence 2023 | U42404 |
| Sequence 2024 | U42404 |
| Sequence 2025 | X51473 |
| Sequence 2026 | 029643 |
| Sequence 2027 | K00799 |
| Sequence 2028 | X79535 |
| Sequence 2029 | X79535 |
| Sequence 2030 | AF020797 |
| Sequence 2031 | AB002533 |
| Sequence 2032 | AB002533 |
| Sequence 2033 | M69181 |
| Sequence 2034 | M69181 |
| Sequence 2035 | M94345 |
| Sequence 2036 | AF029890 |
| Sequence 2037 | AF029890 |
| Sequence 2038 | X56998 |
| Sequence 2039 | X56998 |
| Sequence 2040 | 087665 |
| Sequence 2041 | 087665 |
| Sequence 2042 | J00127 |
| Sequence 2043 | M58569 |
| Sequence 2044 | M16247 |
| Sequence 2045 | M16247 |
| Sequence 2046 | X69392 |
| Sequence 2047 | U73778 |
| Sequence 2048 | U42457 |
| Sequence 2049 | 016892 |
| Sequence 2050 | X15187 |
| Sequence 2051 | M14083 |
| Sequence 2052 | Z82022 |

37/56

| | |
|---|---|
| Sequence 2053 | D89667 |
| Sequence 2054 | J03460 |
| Sequence 2055 | J02611 |
| Sequence 2056 | AF145316 |
| Sequence 2057 | U42404 |
| Sequence 2058 | U42404 |
| Sequence 2059 | AF070561 |
| Sequence 2060 | AF070561 |
| Sequence 2061 | AF070649 |
| Sequence 2062 | AB019568 |
| Sequence 2063 | J03634 |
| Sequence 2064 | J03634 |
| Sequence 2065 | AJ010841 |
| Sequence 2066 | D37766 |
| Sequence 2067 | AF054990 |
| Sequence 2068 | X79535 |
| Sequence 2069 | U05598 |
| Sequence 2070 | AF007791 |
| Sequence 2071 | AF038451 |
| Sequence 2072 | J03592 |
| Sequence 2073 | X87176 |
| Sequence 2074 | U42404 |
| Sequence 2075 | U42594 |
| Sequence 2076 | D29992 |
| Sequence 2077 | AL050218 |
| Sequence 2078 | AL021683 |
| Sequence 2079 | M10119 |
| Sequence 2080 | M10119 |
| Sequence 2081 | D87454 |
| Sequence 2082 | D87454 |
| Sequence 2083 | AB019568 |
| Sequence 2084 | M22920 |
| Sequence 2085 | X07979 |
| Sequence 2086 | AL050209 |
| Sequence 2087 | AL050209 |
| Sequence 2088 | AB014577 |
| Sequence 2089 | AB014577 |
| Sequence 2090 | AJ010841 |
| Sequence 2091 | AF045606 |
| Sequence 2092 | AF086280 |
| Sequence 2093 | AB014548 |
| Sequence 2094 | AF132959 |
| Sequence 2095 | D50918 |
| Sequence 2096 | X87176 |
| Sequence 2097 | M24630 |
| Sequence 2098 | EQ1650 |
| Sequence 2099 | M69181 |
| Sequence 2100 | AJ010841 |
| Sequence 2101 | AB012664 |
| Sequence 2102 | J03202 |
| Sequence 2103 | AF017789 |
| Sequence 2104 | AF017789 |
| Sequence 2105 | X56999 |
| Sequence 2106 | AF035319 |
| Sequence 2107 | K03515 |
| Sequence 2108 | U39318 |
| Sequence 2109 | X87176 |

38/56

| | |
|---|---|
| Sequence 2110 | X87176 |
| Sequence 2111 | AB007896 |
| Sequence 2112 | AF001434 |
| Sequence 2113 | AB007896 |
| Sequence 2114 | AB007896 |
| Sequence 2115 | D50525 |
| Sequence 2116 | Z24725 |
| Sequence 2117 | AB000220 |
| Sequence 2118 | AB000220 |
| Sequence 2119 | D16234 |
| Sequence 2120 | D16234 |
| Sequence 2121 | M55265 |
| Sequence 2122 | J03779 |
| Sequence 2123 | U90545 |
| Sequence 2124 | AF026166 |
| Sequence 2125 | E03157 |
| Sequence 2126 | X60489 |
| Sequence 2127 | AB007883 |
| Sequence 2128 | AB007883 |
| Sequence 2129 | M10905 |
| Sequence 2130 | AF026166 |
| Sequence 2131 | X63692 |
| Sequence 2132 | K00799 |
| Sequence 2133 | X15187 |
| Sequence 2134 | X15187 |
| Sequence 2135 | X63692 |
| Sequence 2136 | K00799 |
| Sequence 2137 | M10905 |
| Sequence 2138 | AB011128 |
| Sequence 2139 | X02761 |
| Sequence 2140 | L27211 |
| Sequence 2141 | AF077043 |
| Sequence 2142 | E01797 |
| Sequence 2143 | J04031 |
| Sequence 2144 | AB011128 |
| Sequence 2145 | X76302 |
| Sequence 2146 | D89053 |
| Sequence 2147 | M24194 |
| Sequence 2148 | U64898 |
| Sequence 2149 | X93207 |
| Sequence 2150 | M10905 |
| Sequence 2151 | J03202 |
| Sequence 2152 | D89053 |
| Sequence 2153 | D13642 |
| Sequence 2154 | J03537 |
| Sequence 2155 | D64015 |
| Sequence 2156 | D64015 |
| Sequence 2157 | AF000421 |
| Sequence 2158 | AF000421 |
| Sequence 2159 | M22920 |
| Sequence 2160 | M22918 |
| Sequence 2161 | D29992 |
| Sequence 2162 | D29992 |
| Sequence 2163 | J03202 |
| Sequence 2164 | J03202 |
| Sequence 2165 | M17885 |
| Sequence 2166 | AF046025 |

39/56

| | |
|---|---|
| Sequence 2167 | AF046025 |
| Sequence 2168 | J02943 |
| Sequence 2169 | J02943 |
| Sequence 2170 | AL021683 |
| Sequence 2171 | J03040 |
| Sequence 2172 | AJ010841 |
| Sequence 2173 | AJ010841 |
| Sequence 2174 | U05598 |
| Sequence 2175 | AB021654 |
| Sequence 2176 | J02943 |
| Sequence 2177 | D38255 |
| Sequence 2178 | D84212 |
| Sequence 2179 | AF008551 |
| Sequence 2180 | L33930 |
| Sequence 2181 | J03202 |
| Sequence 2182 | J03202 |
| Sequence 2183 | Z22555 |
| Sequence 2184 | K00799 |
| Sequence 2185 | X02761 |
| Sequence 2186 | X63432 |
| Sequence 2187 | X51473 |
| Sequence 2188 | X81198 |
| Sequence 2189 | D13388 |
| Sequence 2190 | AB018346 |
| Sequence 2191 | X70326 |
| Sequence 2192 | M23254 |
| Sequence 2193 | AF086003 |
| Sequence 2194 | K00799 |
| Sequence 2195 | X67698 |
| Sequence 2196 | X67698 |
| Sequence 2197 | M10941 |
| Sequence 2198 | EQ1650 |
| Sequence 2199 | AF086336 |
| Sequence 2200 | AJ011007 |
| Sequence 2201 | U76421 |
| Sequence 2202 | D29992 |
| Sequence 2203 | AB001106 |
| Sequence 2204 | J00129 |
| Sequence 2205 | AF052164 |
| Sequence 2206 | U61836 |
| Sequence 2207 | K02765 |
| Sequence 2208 | AB006679 |
| Sequence 2209 | M10905 |
| Sequence 2210 | AF077045 |
| Sequence 2211 | AF089747 |
| Sequence 2212 | J03460 |
| Sequence 2213 | J03460 |
| Sequence 2214 | AB007862 |
| Sequence 2215 | AB007862 |
| Sequence 2216 | AB018346 |
| Sequence 2217 | AB019568 |
| Sequence 2218 | AF000982 |
| Sequence 2219 | J00127 |
| Sequence 2220 | D45198 |
| Sequence 2221 | M15990 |
| Sequence 2222 | M15990 |
| Sequence 2223 | AB010812 |

40/56

| | |
|---|---|
| Sequence 2224 | U63139 |
| Sequence 2225 | AF028832 |
| Sequence 2226 | AF145316 |
| Sequence 2227 | EQ1816 |
| Sequence 2228 | Y00281 |
| Sequence 2229 | AF035752 |
| Sequence 2230 | U94586 |
| Sequence 2231 | AB019568 |
| Sequence 2232 | J04823 |
| Sequence 2233 | L43964 |
| Sequence 2234 | L43964 |
| Sequence 2235 | M31523 |
| Sequence 2236 | AF007128 |
| Sequence 2237 | AF023611 |
| Sequence 2238 | AF031385 |
| Sequence 2239 | L37368 |
| Sequence 2240 | J03460 |
| Sequence 2241 | L13286 |
| Sequence 2242 | AL050380 |
| Sequence 2243 | AL050380 |
| Sequence 2244 | U33760 |
| Sequence 2245 | AB019564 |
| Sequence 2246 | M27504 |
| Sequence 2247 | M27504 |
| Sequence 2248 | AF030555 |
| Sequence 2249 | AF030555 |
| Sequence 2250 | M37583 |
| Sequence 2251 | AF047002 |
| Sequence 2252 | AF086513 |
| Sequence 2253 | AJ002030 |
| Sequence 2254 | U85755 |
| Sequence 2255 | L05425 |
| Sequence 2256 | L05425 |
| Sequence 2257 | X07897 |
| Sequence 2258 | X07897 |
| Sequence 2259 | K03515 |
| Sequence 2260 | K03515 |
| Sequence 2261 | X70394 |
| Sequence 2262 | X70394 |
| Sequence 2263 | M10905 |
| Sequence 2264 | U41850 |
| Sequence 2265 | L22154 |
| Sequence 2266 | AJ004913 |
| Sequence 2267 | AF064093 |
| Sequence 2268 | S80343 |
| Sequence 2269 | D13641 |
| Sequence 2270 | D13641 |
| Sequence 2271 | AF016535 |
| Sequence 2272 | S68330 |
| Sequence 2273 | L05425 |
| Sequence 2274 | AF054987 |
| Sequence 2275 | AF054987 |
| Sequence 2276 | AL049386 |
| Sequence 2277 | AB019564 |
| Sequence 2278 | AF054990 |
| Sequence 2279 | AB007931 |
| Sequence 2280 | M74524 |

41/56

| | |
|---|---|
| Sequence 2281 | U76764 |
| Sequence 2282 | X05908 |
| Sequence 2283 | J02943 |
| Sequence 2284 | AF117815 |
| Sequence 2285 | J03537 |
| Sequence 2286 | AF070550 |
| Sequence 2287 | J00127 |
| Sequence 2288 | U42594 |
| Sequence 2289 | J03460 |
| Sequence 2290 | X53331 |
| Sequence 2291 | M58549 |
| Sequence 2292 | M81757 |
| Sequence 2293 | M86667 |
| Sequence 2294 | L06328 |
| Sequence 2295 | L08666 |
| Sequence 2296 | AJ011007 |
| Sequence 2297 | X07897 |
| Sequence 2298 | AF035752 |
| Sequence 2299 | AF077205 |
| Sequence 2300 | D13748 |
| Sequence 2301 | D13748 |
| Sequence 2302 | X63527 |
| Sequence 2303 | 014665 |
| Sequence 2304 | D14665 |
| Sequence 2305 | X64707 |
| Sequence 2306 | AF132000 |
| Sequence 2307 | AF132000 |
| Sequence 2308 | X87949 |
| Sequence 2309 | M31899 |
| Sequence 2310 | M31899 |
| Sequence 2311 | X63432 |
| Sequence 2312 | V00478 |
| Sequence 2313 | U42594 |
| Sequence 2314 | U42594 |
| Sequence 2315 | AF039656 |
| Sequence 2316 | U84573 |
| Sequence 2317 | U84573 |
| Sequence 2318 | 055654 |
| Sequence 2319 | AL050161 |
| Sequence 2320 | AL050380 |
| Sequence 2321 | U34360 |
| Sequence 2322 | AJ010841 |
| Sequence 2323 | AB020684 |
| Sequence 2324 | AB020684 |
| Sequence 2325 | U63139 |
| Sequence 2326 | U63139 |
| Sequence 2327 | M80359 |
| Sequence 2328 | M80359 |
| Sequence 2329 | X97065 |
| Sequence 2330 | X97065 |
| Sequence 2331 | U94586 |
| Sequence 2332 | U05598 |
| Sequence 2333 | AF070672 |
| Sequence 2334 | AF070672 |
| Sequence 2335 | AF098865 |
| Sequence 2336 | AF098865 |
| Sequence 2337 | AF070561 |

42/56

| | |
|---|---|
| Sequence 2338 | AF070550 |
| Sequence 2339 | AF070550 |
| Sequence 2340 | J03537 |
| Sequence 2341 | AF069072 |
| Sequence 2342 | AF069072 |
| Sequence 2343 | AF038451 |
| Sequence 2344 | AF007791 |
| Sequence 2345 | U77085 |
| Sequence 2346 | AF070561 |
| Sequence 2347 | AF070561 |
| Sequence 2348 | X79535 |
| Sequence 2349 | M10905 |
| Sequence 2350 | AF039656 |
| Sequence 2351 | U39360 |
| Sequence 2352 | U39360 |
| Sequence 2353 | D78335 |
| Sequence 2354 | U76764 |
| Sequence 2355 | AB011108 |
| Sequence 2356 | AL049974 |
| Sequence 2357 | J03202 |
| Sequence 2358 | J03202 |
| Sequence 2359 | AF077043 |
| Sequence 2360 | AF077043 |
| Sequence 2361 | U84573 |
| Sequence 2362 | U02032 |
| Sequence 2363 | Z28407 |
| Sequence 2364 | K00799 |
| Sequence 2365 | U25766 |
| Sequence 2366 | U25766 |
| Sequence 2367 | AB017363 |
| Sequence 2368 | J03537 |
| Sequence 2369 | L31610 |
| Sequence 2370 | S60099 |
| Sequence 2371 | AB007862 |
| Sequence 2372 | AB007862 |
| Sequence 2373 | AF031385 |
| Sequence 2374 | J02814 |
| Sequence 2375 | J02814 |
| Sequence 2376 | U79291 |
| Sequence 2377 | AF077045 |
| Sequence 2378 | AF077045 |
| Sequence 2379 | M94345 |
| Sequence 2380 | AF064093 |
| Sequence 2381 | AF064093 |
| Sequence 2382 | X04098 |
| Sequence 2383 | J00129 |
| Sequence 2384 | M58569 |
| Sequence 2385 | AB020660 |
| Sequence 2386 | M94345 |
| Sequence 2387 | J04991 |
| Sequence 2388 | AB014577 |
| Sequence 2389 | AL049339 |
| Sequence 2390 | AB014512 |
| Sequence 2391 | X13709 |
| Sequence 2392 | M19961 |
| Sequence 2393 | M22920 |
| Sequence 2394 | K00799 |

43/56

| | |
|---|---|
| Sequence 2395 | X02761 |
| Sequence 2396 | S75169 |
| Sequence 2397 | AF085361 |
| Sequence 2398 | J00127 |
| Sequence 2399 | M24486 |
| Sequence 2400 | J03460 |
| Sequence 2401 | M10119 |
| Sequence 2402 | E02628 |
| Sequence 2403 | E01650 |
| Sequence 2404 | J03202 |
| Sequence 2405 | J03202 |
| Sequence 2406 | U41515 |
| Sequence 2407 | M24630 |
| Sequence 2408 | M24630 |
| Sequence 2409 | S59184 |
| Sequence 2410 | X69970 |
| Sequence 2411 | D50312 |
| Sequence 2412 | D50312 |
| Sequence 2413 | AB000220 |
| Sequence 2414 | AB000220 |
| Sequence 2415 | D87453 |
| Sequence 2416 | U14391 |
| Sequence 2417 | U14391 |
| Sequence 2418 | AF073298 |
| Sequence 2419 | AF086336 |
| Sequence 2420 | AF086336 |
| Sequence 2421 | AF000982 |
| Sequence 2422 | AF000982 |
| Sequence 2423 | U19252 |
| Sequence 2424 | U19252 |
| Sequence 2425 | J03799 |
| Sequence 2426D | 50371 |
| Sequence 2427 | U12404 |
| Sequence 2428 | AF086557 |
| Sequence 2429 | X56932 |
| Sequence 2430 | J02943 |
| Sequence 2431 | D50371 |
| Sequence 2432 | AB028624 |
| Sequence 2433 | M65217 |
| Sequence 2434 | U30246 |
| Sequence 2435 | U30246 |
| Sequence 2436 | X02761 |
| Sequence 2437 | AB002533 |
| Sequence 2438 | AB002533 |
| Sequence 2439 | D49737 |
| Sequence 2440 | X07897 |
| Sequence 2441 | M55268 |
| Sequence 2442 | S66431 |
| Sequence 2443 | S66431 |
| Sequence 2444 | U00947 |
| Sequence 2445 | M33308 |
| Sequence 2446 | M33308 |
| Sequence 2447 | J04543 |
| Sequence 2448 | M33308 |
| Sequence 2449 | M10905 |
| Sequence 2450 | L16785 |
| Sequence 2451 | L16785 |

44/56

| | |
|---|---|
| Sequence 2452 | U63139 |
| Sequence 2453 | U63139 |
| Sequence 2454 | AF088071 |
| Sequence 2455 | U15008 |
| Sequence 2456 | X04098 |
| Sequence 2457 | X04098 |
| Sequence 2458 | D63476 |
| Sequence 2459 | X79535 |
| Sequence 2460 | D50918 |
| Sequence 2461 | D50918 |
| Sequence 2462 | U42404 |
| Sequence 2463 | D13641 |
| Sequence 2464 | D13641 |
| Sequence 2465 | AB023151 |
| Sequence 2466 | AB023151 |
| Sequence 2467 | U42594 |
| Sequence 2468 | U42594 |
| Sequence 2469 | U42457 |
| Sequence 2470 | EQ1816 |
| Sequence 2471 | U42404 |
| Sequence 2472 | U42404 |
| Sequence 2473 | AF086336 |
| Sequence 2474 | AF086336 |
| Sequence 2475 | U42404 |
| Sequence 2476 | J04621 |
| Sequence 2477 | AF039656 |
| Sequence 2478 | D50918 |
| Sequence 2479 | AF001601 |
| Sequence 2480 | AF001601 |
| Sequence 2481 | AB014577 |
| Sequence 2482 | AB014577 |
| Sequence 2483 | AF054183 |
| Sequence 2484 | AF052578 |
| Sequence 2485 | M12623 |
| Sequence 2486 | AF151832 |
| Sequence 2487 | AF007170 |
| Sequence 2488 | AF007170 |
| Sequence 2489 | AF054987 |
| Sequence 2490 | AF054987 |
| Sequence 2491 | J02943 |
| Sequence 2492 | L20941 |
| Sequence 2493 | L20941 |
| Sequence 2494 | M27504 |
| Sequence 2495 | M27504 |
| Sequence 2496 | AF104419 |
| Sequence 2497 | M80359 |
| Sequence 2498 | M80359 |
| Sequence 2499 | AF039656 |
| Sequence 2500 | U57847 |
| Sequence 2501 | U57847 |
| Sequence 2502 | D13642 |
| Sequence 2503 | X02761 |
| Sequence 2504 | Y08982 |
| Sequence 2505 | AJ010841 |
| Sequence 2506 | AJ002030 |
| Sequence 2507 | AJ002030 |
| Sequence 2508 | AB019568 |

| | |
|---|---|
| 45/56 | |
| Sequence 2509 | AB021654 |
| Sequence 2510 | U05598 |
| Sequence 2511 | AF099149 |
| Sequence 2512 | L37368 |
| Sequence 2513 | L37368 |
| Sequence 2514 | AB019564 |
| Sequence 2515 | J00128 |
| Sequence 2516 | J00127 |
| Sequence 2517 | D90209 |
| Sequence 2518 | D90209 |
| Sequence 2519 | AB007862 |
| Sequence 2520 | AB007862 |
| Sequence 2521 | E01954 |
| Sequence 2522 | E01954 |
| Sequence 2523 | AB020692 |
| Sequence 2524 | AB020692 |
| Sequence 2525 | J04973 |
| Sequence 2526 | D63486 |
| Sequence 2527 | D45906 |
| Sequence 2528 | J00127 |
| Sequence 2529 | J00127 |
| Sequence 2530 | U12465 |
| Sequence 2531 | U12465 |
| Sequence 2532 | K03515 |
| Sequence 2533 | AB020662 |
| Sequence 2534 | AB002322 |
| Sequence 2535 | K03515 |
| Sequence 2536 | K03515 |
| Sequence 2537 | U30246 |
| Sequence 2538 | U30246 |
| Sequence 2539 | AF147331 |
| Sequence 2540 | X00570 |
| Sequence 2541 | AF077599 |
| Sequence 2542 | J00129 |
| Sequence 2543 | J00129 |
| Sequence 2544 | U42404 |
| Sequence 2545 | U42404 |
| Sequence 2546 | AJ002030 |
| Sequence 2547 | AJ002030 |
| Sequence 2548 | AB023195 |
| Sequence 2549 | AB023195 |
| Sequence 2550 | D50310 |
| Sequence 2551 | U39402 |
| Sequence 2552 | X87949 |
| Sequence 2553 | M10119 |
| Sequence 2554 | M10119 |
| Sequence 2555 | AF070550 |
| Sequence 2556 | AF070550 |
| Sequence 2557 | AF131781 |
| Sequence 2558 | AF131781 |
| Sequence 2559 | U53476 |
| Sequence 2560 | M10905 |
| Sequence 2561 | X02761 |
| Sequence 2562 | Y00503 |
| Sequence 2563 | J03040 |
| Sequence 2564 | U42404 |
| Sequence 2565 | U42404 |
| 46/56 | |

| | |
|---|---|
| Sequence 2566 | AF092128 |
| Sequence 2567 | J03796 |
| Sequence 2568 | J03796 |
| Sequence 2569 | AF070606 |
| Sequence 2570 | M17885 |
| Sequence 2571 | D63486 |
| Sequence 2572 | D63486 |
| Sequence 2573 | D87127 |
| Sequence 2574 | D87127 |
| Sequence 2575 | J02943 |
| Sequence 2576 | J02943 |
| Sequence 2577 | AB001106 |
| Sequence 2578 | M10905 |
| Sequence 2579 | D45906 |
| Sequence 2580 | AF092133 |
| Sequence 2581 | J03537 |
| Sequence 2582 | M58485 |
| Sequence 2583 | AF077205 |
| Sequence 2584 | AF077205 |
| Sequence 2585 | AF007791 |
| Sequence 2586 | AF007791 |
| Sequence 2587 | D49737 |
| Sequence 2588 | D49737 |
| Sequence 2589 | X15880 |
| Sequence 2590 | X77588 |
| Sequence 2591 | U03272 |
| Sequence 2592 | AF0086336 |
| Sequence 2593 | D49489 |
| Sequence 2594 | S78085 |
| Sequence 2595 | X00570 |
| Sequence 2596 | J03460 |
| Sequence 2597 | AF007791 |
| Sequence 2598 | U77085 |
| Sequence 2599 | U77085 |
| Sequence 2600 | X07979 |
| Sequence 2601 | D63776 |
| Sequence 2602 | D83776 |
| Sequence 2603 | AF086336 |
| Sequence 2604 | AF086336 |
| Sequence 2605 | AF026004 |
| Sequence 2606 | AF077044 |
| Sequence 2607 | AF077044 |
| Sequence 2608 | AF151856 |
| Sequence 2609 | AF151856 |
| Sequence 2610 | AB018346 |
| Sequence 2611 | AB018346 |
| Sequence 2612 | U78575 |
| Sequence 2613 | U78578 |
| Sequence 2614 | M10905 |
| Sequence 2615 | K00799 |
| Sequence 2616 | K00799 |
| Sequence 2617 | L05425 |
| Sequence 2618 | L05425 |
| Sequence 2619 | AF086336 |
| Sequence 2620 | AJ011007 |
| Sequence 2621 | L19184 |
| Sequence 2622 | X67951 |

47/56

| | |
|---|---|
| Sequence 2623 | AF038451 |
| Sequence 2624 | M29960 |
| Sequence 2625 | AF086336 |
| Sequence 2626 | AF086336 |
| Sequence 2627 | Y00345 |
| Sequence 2628 | Y00345 |
| Sequence 2629 | J03202 |
| Sequence 2630 | AF086183 |
| Sequence 2631 | AF086183 |
| Sequence 2632 | X77588 |
| Sequence 2633 | U14528 |
| Sequence 2634 | X17206 |
| Sequence 2635 | M81757 |
| Sequence 2636 | M81757 |
| Sequence 2637 | J03202 |
| Sequence 2638 | J03202 |
| Sequence 2639 | AF007791 |
| Sequence 2640 | AF038451 |
| Sequence 2641 | AF007791 |
| Sequence 2642 | M98479 |
| Sequence 2643 | E01650 |
| Sequence 2644 | AF114264 |
| Sequence 2645 | U42404 |
| Sequence 2646 | U42404 |
| Sequence 2647 | AB002533 |
| Sequence 2648 | Y08982 |
| Sequence 2649 | AF086172 |
| Sequence 2650 | X02761 |
| Sequence 2651 | U30521 |
| Sequence 2652 | U30521 |
| Sequence 2653 | X63527 |
| Sequence 2654 | L05092 |
| Sequence 2655 | L05092 |
| Sequence 2656 | Z24725 |
| Sequence 2657 | X54942 |
| Sequence 2658 | X54942 |
| Sequence 2659 | AB002439 |
| Sequence 2660 | X81889 |
| Sequence 2661 | X81889 |
| Sequence 2662 | AF007150 |
| Sequence 2663 | L10910 |
| Sequence 2664 | AL049339 |
| Sequence 2665 | X55740 |
| Sequence 2666 | X55740 |
| Sequence 2667 | AF143096 |
| Sequence 2668 | AF143096 |
| Sequence 2669 | AB020684 |
| Sequence 2670 | U42457 |
| Sequence 2671 | Z31696 |
| Sequence 2672 | AF098865 |
| Sequence 2673 | AF098865 |
| Sequence 2674 | U12404 |
| Sequence 2675 | S75169 |
| Sequence 2676 | AF070672 |
| Sequence 2677 | AF153329 |
| Sequence 2678 | U05040 |
| Sequence 2679 | AF086336 |

48/56

| | |
|---|---|
| Sequence 2680 | AF132959 |
| Sequence 2681 | U47077 |
| Sequence 2682 | U34994 |
| Sequence 2683 | J03460 |
| Sequence 2684 | U06452 |
| Sequence 2685 | U06452 |
| Sequence 2686 | M34788 |
| Sequence 2687 | M34788 |
| Sequence 2688 | 021260 |
| Sequence 2689 | D21260 |
| Sequence 2690 | AF039575 |
| Sequence 2691 | D55671 |
| Sequence 2692 | X16312 |
| Sequence 2693 | J03202 |
| Sequence 2694 | J03202 |
| Sequence 2695 | AF077045 |
| Sequence 2696 | D29992 |
| Sequence 2697 | AF070606 |
| Sequence 2698 | AF070606 |
| Sequence 2699 | K00799 |
| Sequence 2700 | K00799 |
| Sequence 2701 | X07979 |
| Sequence 2702 | E02628 |
| Sequence 2703 | AF016582 |
| Sequence 2704 | AF016582 |
| Sequence 2705 | U47101 |
| Sequence 2706 | U47101 |
| Sequence 2707 | L05425 |
| Sequence 2708 | AJ010841 |
| Sequence 2709 | U70660 |
| Sequence 2710 | AF052178 |
| Sequence 2711 | M98479 |
| Sequence 2712 | AF151856 |
| Sequence 2713 | AF151856 |
| Sequence 2714 | AF147331 |
| Sequence 2715 | K00799 |
| Sequence 2716 | K00799 |
| Sequence 2717 | 063486 |
| Sequence 2718 | 063486 |
| Sequence 2719 | D50371 |
| Sequence 2720 | AB028624 |
| Sequence 2721 | AF020797 |
| Sequence 2722 | AB014577 |
| Sequence 2723 | AB014577 |
| Sequence 2724 | AB014512 |
| Sequence 2725 | M69181 |
| Sequence 2726 | M69181 |
| Sequence 2727 | U42404 |
| Sequence 2728 | U42404 |
| Sequence 2729 | 013641 |
| Sequence 2730 | D13641 |
| Sequence 2731 | D14657 |
| Sequence 2732 | L16785 |
| Sequence 2733 | 087469 |
| Sequence 2734 | X13709 |
| Sequence 2735 | M19961 |
| Sequence 2736 | AL049974 |

49/56

| | |
|---|---|
| Sequence 2737 | M19961 |
| Sequence 2738 | X02761 |
| Sequence 2739 | Y08982 |
| Sequence 2740 | X96484 |
| Sequence 2741 | AL049339 |
| Sequence 2742 | AF114264 |
| Sequence 2743 | AF114264 |
| Sequence 2744 | Z13009 |
| Sequence 2745 | AB002330 |
| Sequence 2746 | AF092128 |
| Sequence 2747 | AF086408 |
| Sequence 2748 | M24486 |
| Sequence 2749 | M24486 |
| Sequence 2750 | AB020657 |
| Sequence 2751 | D87667 |
| Sequence 2752 | D87667 |
| Sequence 2753 | X15187 |
| Sequence 2754 | M81757 |
| Sequence 2755 | M38188 |
| Sequence 2756 | AJ007669 |
| Sequence 2757 | M64098 |
| Sequence 2758 | J02943 |
| Sequence 2759 | AB002439 |
| Sequence 2760 | S72481 |
| Sequence 2761 | S72481 |
| Sequence 2762 | D00099 |
| Sequence 2763 | D00099 |
| Sequence 2764 | M10905 |
| Sequence 2765 | M14060 |
| Sequence 2766 | Z31696 |
| Sequence 2767 | X81109 |
| Sequence 2768 | J03202 |
| Sequence 2769 | U46194 |
| Sequence 2770 | AB018281 |
| Sequence 2771 | X59618 |
| Sequence 2772 | AF016582 |
| Sequence 2773 | X79535 |
| Sequence 2774 | L13806 |
| Sequence 2775 | U75283 |
| Sequence 2776 | Y00052 |
| Sequence 2777 | M23254 |
| Sequence 2778 | X69141 |
| Sequence 2779 | J03202 |
| Sequence 2780 | K00799 |
| Sequence 2781 | AF068706 |
| Sequence 2782 | J02814 |
| Sequence 2783 | K00799 |
| Sequence 2784 | AB021654 |
| Sequence 2785 | AF016535 |
| Sequence 2786 | M10905 |
| Sequence 2787 | AB007862 |
| Sequence 2788 | U46194 |
| Sequence 2789 | AF070550 |
| Sequence 2790 | J03796 |
| Sequence 2791 | X04098 |
| Sequence 2792 | U30246 |
| Sequence 2793 | U25789 |

50/56

| | |
|---|---|
| Sequence 2794 | AF086336 |
| Sequence 2795 | M22918 |
| Sequence 2796 | M10905 |
| Sequence 2797 | U25766 |
| Sequence 2798 | X79535 |
| Sequence 2799 | L16785 |
| Sequence 2800 | L16785 |
| Sequence 2801 | M22636 |
| Sequence 2802 | M22636 |
| Sequence 2803 | 087735 |
| Sequence 2804 | X07979 |
| Sequence 2805 | U05598 |
| Sequence 2806 | AB021654 |
| Sequence 2807 | X17206 |
| Sequence 2808 | 087735 |
| Sequence 2809 | J04088 |
| Sequence 2810 | AF046025 |
| Sequence 2811 | AF046025 |
| Sequence 2812 | AF086336 |
| Sequence 2813 | AJQ11007 |
| Sequence 2814 | X02761 |
| Sequence 2815 | X02761 |
| Sequence 2816 | Y00819 |
| Sequence 2817 | X04098 |
| Sequence 2818 | X04098 |
| Sequence 2819 | AF086205 |
| Sequence 2820 | X85373 |
| Sequence 2821 | X85373 |
| Sequence 2822 | L12387 |
| Sequence 2823 | L12387 |
| Sequence 2824 | AF054990 |
| Sequence 2825 | AL049929 |
| Sequence 2826 | K00799 |
| Sequence 2827 | U42404 |
| Sequence 2828 | AF086336 |
| Sequence 2829 | AF086336 |
| Sequence 2830 | K00799 |
| Sequence 2831 | 029992 |
| Sequence 2832 | 029992 |
| Sequence 2833 | U31384 |
| Sequence 2834 | U31384 |
| Sequence 2835 | AF086336 |
| Sequence 2836 | AF086336 |
| Sequence 2837 | AF077043 |
| Sequence 2838 | AF043431 |
| Sequence 2839 | AF043431 |
| Sequence 2840 | M17885 |
| Sequence 2841 | U73377 |
| Sequence 2842 | X70394 |
| Sequence 2843 | X70394 |
| Sequence 2844 | X00351 |
| Sequence 2845 | X63432 |
| Sequence 2846 | X02308 |
| Sequence 2847 | M10905 |
| Sequence 2848 | M10905 |
| Sequence 2849 | Z26317 |
| Sequence 2850 | Z26317 |

51/56

| | |
|---|---|
| Sequence 2851 | M10905 |
| Sequence 2852 | M22918 |
| Sequence 2853 | AF011793 |
| Sequence 2854 | AJ001309 |
| Sequence 2855 | AF068706 |
| Sequence 2856 | X67731 |
| Sequence 2857 | AF016535 |
| Sequence 2858 | AF016535 |
| Sequence 2859 | X02761 |
| Sequence 2860 | AB007883 |
| Sequence 2861 | AB011089 |
| Sequence 2862 | L13806 |
| Sequence 2863 | M10905 |
| Sequence 2864 | L05425 |
| Sequence 2865 | L05425 |
| Sequence 2866 | 029992 |
| Sequence 2867 | AF007791 |
| Sequence 2868 | 083776 |
| Sequence 2869 | 083776 |
| Sequence 2870 | AB014577 |
| Sequence 2871 | AB014577 |
| Sequence 2872 | X55740 |
| Sequence 2873 | X55740 |
| Sequence 2874 | L05425 |
| Sequence 2875 | AB017116 |
| Sequence 2876 | AB017116 |
| Sequence 2877 | K00799 |
| Sequence 2878 | U42404 |
| Sequence 2879 | U42404 |
| Sequence 2880 | J03198 |
| Sequence 2881 | J03198 |
| Sequence 2882 | M58549 |
| Sequence 2883 | 029992 |
| Sequence 2884 | AB007883 |
| Sequence 2885 | AB007883 |
| Sequence 2886 | M33308 |
| Sequence 288T | M33308 |
| Sequence 2888 | 087735 |
| Sequence 2889 | 087735 |
| Sequence 2890 | AB011128 |
| Sequence 2891 | AJ010841 |
| Sequence 2892 | AJ010841 |
| Sequence 2893 | U42404 |
| Sequence 2894 | U42404 |
| Sequence 2895 | M23114 |
| Sequence 2896 | S75169 |
| Sequence 2897 | S75169 |
| Sequence 2898 | X02761 |
| Sequence 2899 | AF007791 |
| Sequence 2900 | K00799 |
| Sequence 2901 | K00799 |
| Sequence 2902 | AF072928 |
| Sequence 2903 | K00799 |
| Sequence 2904 | U76421 |
| Sequence 2905 | M58549 |
| Sequence 2906 | X02761 |
| Sequence 2907 | X02761 |

52/56

| | |
|---|---|
| Sequence 2908 | M69181 |
| Sequence 2909 | M69181 |
| Sequence 2910 | AB019568 |
| Sequence 2911 | AF031385 |
| Sequence 2912 | X04470 |
| Sequence 2913 | X04470 |
| Sequence 2914 | U76421 |
| Sequence 2915 | AB023191 |
| Sequence 2916 | AB023191 |
| Sequence 2917 | U34994 |
| Sequence 2918 | U34994 |
| Sequence 2919 | X84407 |
| Sequence 2920 | AJ011001 |
| Sequence 2921 | AJ011001 |
| Sequence 2922 | 013748 |
| Sequence 2923 | D13748 |
| Sequence 2924 | 029992 |
| Sequence 2925 | D29992 |
| Sequence 2926 | U42457 |
| Sequence 2927 | U42457 |
| Sequence 2928 | AL050282 |
| Sequence 2929 | EQ1650 |
| Sequence 2930 | J03779 |
| Sequence 2931 | K00799 |
| Sequence 2932 | K00799 |
| Sequence 2933 | AF085844 |
| Sequence 2934 | M16462 |
| Sequence 2935 | Y09565 |
| Sequence 2936 | D87667 |
| Sequence 2937 | 087667 |
| Sequence 2938 | L05093 |
| Sequence 2939 | AB007883 |
| Sequence 2940 | U14971 |
| Sequence 2941 | U14971 |
| Sequence 2942 | U44754 |
| Sequence 2943 | U44754 |
| Sequence 2944 | X60656 |
| Sequence 2945 | AB018346 |
| Sequence 2946 | U85658 |
| Sequence 2947 | U85658 |
| Sequence 2948 | M69181 |
| Sequence 2949 | AF008551 |
| Sequence 2950 | AF008551 |
| Sequence 2951 | D29992 |
| Sequence 2952 | S77362 |
| Sequence 2953 | J05032 |
| Sequence 2954 | AF052178 |
| Sequence 2955 | AF052178 |
| Sequence 2956 | AJ010841 |
| Sequence 2957 | AJ010841 |
| Sequence 2958 | U02032 |
| Sequence 2959 | U02032 |
| Sequence 2960 | AB011089 |
| Sequence 2961 | AF050638 |
| Sequence 2962 | 085181 |
| Sequence 2963 | AJ010841 |
| Sequence 2964 | AJ010841 |

53/56

| | |
|---|---|
| Sequence 2965 | U07343 |
| Sequence 2966 | U42404 |
| Sequence 2967 | U42404 |
| Sequence 2968 | X04098 |
| Sequence 2969 | X04098 |
| Sequence 2970 | AF011793 |
| Sequence 2971 | AJ001309 |
| Sequence 2972 | AL049339 |
| Sequence 2973 | AF028832 |
| Sequence 2974 | AF131797 |
| Sequence 2975 | U42404 |
| Sequence 2976 | U42404 |
| Sequence 2977 | X02761 |
| Sequence 2978 | AB028974 |
| Sequence 2979 | AB028974 |
| Sequence 2980 | U45976 |
| Sequence 2981 | L22154 |
| Sequence 2982 | AF068007 |
| Sequence 2983 | D83776 |
| Sequence 2984 | D83776 |
| Sequence 2985 | J02943 |
| Sequence 2986 | J04543 |
| Sequence 2987 | M14200 |
| Sequence 2988 | U42404 |
| Sequence 2989 | U42404 |
| Sequence 2990 | AF077208 |
| Sequence 2991 | J02943 |
| Sequence 2992 | U18062 |
| Sequence 2993 | U18062 |
| Sequence 2994 | AF010309 |
| Sequence 2995 | AF010309 |
| Sequence 2996 | AL132665 |
| Sequence 2997 | AL132665 |
| Sequence 2998 | X69181 |
| Sequence 2999 | J03015 |
| Sequence 3000 | J03015 |
| Sequence 3001 | X81889 |
| Sequence 3002 | D13641 |
| Sequence 3003 | D13641 |
| Sequence 3004 | X96484 |
| Sequence 3005 | U42404 |
| Sequence 3006 | X69970 |
| Sequence 3007 | J02943 |
| Sequence 3008 | M69181 |
| Sequence 3009 | J02943 |
| Sequence 3010 | D83776 |
| Sequence 3011 | D83776 |
| Sequence 3012 | D87667 |
| Sequence 3013 | M73792 |
| Sequence 3014 | X15187 |
| Sequence 3015 | X15187 |
| Sequence 3016 | AF081280 |
| Sequence 3017 | AF086003 |
| Sequence 3018 | AF086003 |
| Sequence 3019 | M11353 |
| Sequence 3020 | M11353 |
| Sequence 3021 | M38188 |

54/56

| | |
|---|---|
| Sequence 3022 | 000099 |
| Sequence 3023 | M19961 |
| Sequence 3024 | M19961 |
| Sequence 3025 | EQ1816 |
| Sequence 3026 | AF100741 |
| Sequence 3027 | AF001176 |
| Sequence 3028 | X04098 |
| Sequence 3029 | U92993 |
| Sequence 3030 | A21185 |
| Sequence 3031 | D17266 |
| Sequence 3032 | AJ002030 |
| Sequence 3033 | AF054179 |
| Sequence 3034 | AF054179 |
| Sequence 3035 | X56932 |
| Sequence 3036 | X69970 |
| Sequence 3037 | X69970 |
| Sequence 3038 | U82130 |
| Sequence 3039 | U82130 |
| Sequence 3040 | M10119 |
| Sequence 3041 | M10119 |
| Sequence 3042 | L16785 |
| Sequence 3043 | AF132959 |
| Sequence 3044 | AF132959 |
| Sequence 3045 | D87667 |
| Sequence 3046 | K00799 |
| Sequence 3047 | M55268 |
| Sequence 3048 | M55268 |
| Sequence 3049 | U42404 |
| Sequence 3050 | X51473 |
| Sequence 3051 | AF016535 |
| Sequence 3052 | L10910 |
| Sequence 3053 | U42404 |
| Sequence 3054 | U42404 |
| Sequence 3055 | D49737 |
| Sequence 3056 | AF086336 |
| Sequence 3057 | X07897 |
| Sequence 3058 | AB011159 |
| Sequence 3059 | AB011159 |
| Sequence 3060 | M81182 |
| Sequence 3061 | M81182 |
| Sequence 3062 | AF046025 |
| Sequence 3063 | AF046025 |
| Sequence 3064 | AB020649 |
| Sequence 3065 | 021260 |
| Sequence 3066 | 021260 |
| Sequence 3067 | 014530 |
| Sequence 3068 | AF038451 |
| Sequence 3069 | 038255 |
| Sequence 3070 | AF008551 |
| Sequence 3071 | AF008551 |
| Sequence 3072 | AF070648 |
| Sequence 3073 | AF026166 |
| Sequence 3074 | U42404 |
| Sequence 3075 | AF047439 |
| Sequence 3076 | AL021683 |
| Sequence 3077 | J04031 |
| Sequence 3078 | M30047 |

55/56

| | |
|---|---|
| Sequence 3079 | Y00819 |
| Sequence 3080 | AB014577 |
| Sequence 3081 | AB014577 |
| Sequence 3082 | D29992 |
| Sequence 3083 | M11353 |
| Sequence 3084 | M11353 |
| Sequence 3085 | AF0B1280 |
| Sequence 3086 | J03202 |
| Sequence 3087 | U42404 |
| Sequence 3088 | U42404 |
| Sequence 3089 | U42404 |
| Sequence 3090 | AF086336 |
| Sequence 3091 | AJ011007 |
| Sequence 3092 | M84739 |
| Sequence 3093 | M84739 |
| Sequence 3094 | AB019568 |
| Sequence 3095 | S80990 |
| Sequence 3096 | AF035812 |
| Sequence 3097 | X02761 |
| Sequence 3098 | AF035319 |
| Sequence 3099 | X28433 |
| Sequence 3100 | N80412 |
| Sequence 3101 | Q66636 |
| Sequence 3102 | V59612 |
| Sequence 3103 | T67164 |
| Sequence 3104 | V40550 |
| Sequence 3105 | V63175 |
| Sequence 3106 | V63175 |
| Sequence 3107 | X28433 |
| Sequence 3108 | V04699 |
| Sequence 3109 | V63175 |
| Sequence 3110 | V46154 |
| Sequence 3111 | X28433 |
| Sequence 3112 | V83134 |
| Sequence 3113 | Q66636 |
| Sequence 3114 | V59663 |
| Sequence 3115 | X04408 |
| Sequence 3116 | Q70007 |
| Sequence 3117 | T15718 |
| Sequence 3118 | V89990 |
| Sequence 3119 | V32416 |
| Sequence 3120 | X28433 |
| Sequence 3121 | X28433 |
| Sequence 3122 | X40333 |
| Sequence 3123 | V59695 |
| Sequence 3124 | Q32364 |
| Sequence 3125 | Q32364 |
| Sequence 3126 | V60015 |
| Sequence 3127 | X28433 |
| Sequence 3128 | X28433 |
| Sequence 3129 | X28433 |
| Sequence 3130 | X28433 |
| Sequence 3131 | A010439 |
| Sequence 3132 | A013954 |

56/56
Sequence 3131: found in patent publication WO99/14328
GCACGCGGTGGCGGCGGCACACTCGTCCACATCCACACAGGC
GCCCTCGTCCAGCACCCAGCCNACTTTACAGTCGCCGCAGTC
TNTGNTGGTCAGGCCCGAGCACGTTTTGCAGGACTCGTTACA
GGNTGTGCAGATGCTGT
Sequence 3132: found in patent publication WO99/24836
AGGTACACTCATCCTGCGTATCATCACTGCCATGTCCTGATA
CCCCAGCTCTGCCATATTGCCCTTCTTTTTTGCGGTATGATG
ACCACATAGAGGCCCAACCTCTTAAACACATCAATACCAATG
ATCACATTTCAATCTAGACTTCTAAGCAACGGCTGAAATCTC
TCCAGGCCAAAGGAGAGTTTGTATCACCTTACCAGAAGCTTC
TCCGGAACAATTGGCCAGAAGCCTAGAGTTCAGAAACCCAGA
CACATGCAGTAAGCAATTTCCAGTTVCTCTATAATTTAGAAG
AGGACACCATGATATGTAATGCGGGGTCTGGGAGGTTGGAAT
GCCTCCATAAAACACCTGCCATATTTTTTGGTCCAAGCCTTA
GTGGTATAAATCAAGAAGGCTGTAAATAAGACTTCAGCTTTT
TGGCTGGTGAAGTTTGGTTCC

TABLE 3

| Acc no. | GI no. |
|---|---|
| AA001696 | 1445252 |
| AA002128 | 1445144 |
| AA009923 | 1470970 |
| AA010575 | 1471742 |
| AA010689 | 1471716 |
| AA010893 | 1471990 |
| AA011002 | 1472029 |
| AA022748 | 1486821 |
| AA022943 | 1487051 |
| AA025349 | 1489317 |
| AA028882 | 1496304 |
| AA034237 | 1506265 |
| AA036750 | 1509788 |
| AA037181 | 1512290 |
| AA040929 | 1515636 |
| AA043103 | 1521053 |
| AA043137 | 1520991 |
| AA045176 | 1523378 |
| AA046473 | 1526403 |
| AA046810 | 1524915 |
| AA046888 | 1524823 |
| AA047054 | 1524952 |
| AA054771 | 1545707 |
| AA056334 | 1548691 |
| AA058936 | 1551791 |
| AA069078 | 1576438 |
| AA069560 | 1576972 |
| AA069850 | 1577210 |
| AA071084 | 1578444 |
| AA074035 | 1613975 |
| AA074291 | 1614159 |
| AA074845 | 1614714 |
| AA075527 | 1615397 |
| AA081348 | 1623162 |
| AA082884 | 1624941 |
| AA083270 | 1625391 |
| AA083410 | 1625660 |
| AA085444 | 1628674 |
| AA085511 | 1628697 |
| AA088344 | 1633856 |
| AA088758 | 1634279 |
| AA095772 | 1641357 |
| AA099904 | 1646050 |
| AA100707 | 1647062 |
| AA101783 | 1648780 |
| AA102138 | 1646213 |
| AA102721 | 1648220 |
| AA102853 | 1648698 |
| AA113420 | 1665269 |
| AA115218 | 1670047 |
| AA115838 | 1670916 |
| AA121574 | 1679249 |
| AA125927 | 1685612 |
| AA127186 | 1686546 |
| AA128091 | 1687353 |
| AA128878 | 1690018 |
| AA128965 | 1688748 |
| AA130823 | 1692515 |
| AA131227 | 1692735 |
| AA132844 | 1694333 |
| AA136789 | 1697998 |
| AA142909 | 1712370 |
| AA143438 | 1712808 |
| AA143746 | 1713134 |
| AA147080 | 1716453 |
| AA149963 | 1721247 |
| AA156443 | 1728068 |
| AA156615 | 1728238 |
| AA156616 | 1728239 |
| AA157300 | 1728926 |
| AA160517 | 1735884 |
| AA161269 | 1735566 |
| AA166632 | 1745096 |
| AA166675 | 1745130 |
| AA167700 | 1744868 |
| AA167814 | 1744965 |
| AA173279 | 1753411 |
| AA174034 | 1754363 |
| AA176813 | 1757945 |
| AA179439 | 1760791 |
| AA181153 | 1764620 |
| AA181811 | 1765337 |
| AA181858 | 1765325 |
| AA187817 | 1774011 |
| AA188680 | 1775705 |
| AA188832 | 1775877 |
| AA191341 | 1780003 |
| AA195178 | 1784909 |
| AA196515 | 1792106 |
| AA199684 | 1795594 |
| AA203284 | 1799010 |
| AA205412 | 1803420 |
| AA211509 | 1810163 |
| AA211584 | 1810247 |
| AA213580 | 1812199 |
| AA215584 | 1815420 |
| AA216094 | 1816041 |
| AA223381 | 1843906 |
| AA224124 | 1844683 |
| AA224163 | 1844731 |
| AA225289 | 1846607 |
| AA226279 | 1847596 |
| AA235063 | 1859499 |
| AA235365 | 1859803 |
| AA251312 | 1886338 |
| AA256442 | 1891980 |
| AA262249 | 1898724 |
| AA278456 | 1919829 |
| AA279497 | 1920962 |
| AA280091 | 1921565 |
| AA281007 | 1923751 |
| AA293450 | 1941151 |
| AA295872 | 1948207 |
| AA300065 | 1952416 |
| AA305272 | 1957598 |
| AA305566 | 1957913 |
| AA305591 | 1957993 |
| AA305876 | 1958206 |
| AA305954 | 1958283 |
| AA306620 | 1958949 |
| AA306660 | 1959213 |
| AA306692 | 1959020 |
| AA306696 | 1959024 |
| AA307818 | 1960145 |
| AA308126 | 1960455 |
| AA308230 | 1960559 |
| AA308374 | 1960703 |
| AA308443 | 1960771 |
| AA308570 | 1960898 |
| AA308801 | 1961131 |
| AA309594 | 1961964 |
| AA309832 | 1962325 |
| AA311573 | 1964057 |
| AA311896 | 1964546 |
| AA312002 | 1964331 |
| AA313534 | 1965864 |
| AA313688 | 1966017 |
| AA314188 | 1966517 |
| AA314196 | 1966525 |
| AA314584 | 1966912 |
| AA314961 | 1967379 |
| AA315889 | 1968217 |
| AA318817 | 1971142 |
| AA325285 | 1977742 |
| AA325809 | 1978052 |
| AA333358 | 1985601 |
| AA344846 | 1997084 |
| AA347752 | 1999987 |
| AA348032 | 2000511 |
| AA350063 | 2002402 |
| AA350719 | 2003036 |
| AA354709 | 2007028 |
| AA355003 | 2007559 |
| AA356654 | 2009197 |
| AA359705 | 2012096 |
| AA361393 | 2013888 |
| AA361953 | 2014274 |
| AA362701 | 2015041 |
| AA367082 | 2019471 |
| AA371964 | 2024282 |
| AA375228 | 2027547 |
| AA377891 | 2030229 |
| AA384315 | 2036634 |
| AA384731 | 2037049 |
| AA400974 | 2054946 |
| AA401759 | 2057243 |
| AA411068 | 2070180 |
| AA416628 | 2077701 |
| AA421632 | 2100448 |
| AA424661 | 2103614 |
| AA425212 | 2106120 |
| AA425260 | 2106034 |
| AA425795 | 2107633 |
| AA427816 | 2111651 |
| AA428014 | 2111742 |
| AA428889 | 2111902 |
| AA443112 | 2155787 |
| AA445966 | 2158631 |
| AA447302 | 3025388 |
| AA447645 | 2161315 |
| AA448559 | 2162229 |
| AA449337 | 2163186 |
| AA453555 | 2167224 |
| AA453632 | 2167301 |
| AA453719 | 2167388 |
| AA454129 | 2167798 |
| AA455807 | 2178583 |
| AA458628 | 2183535 |
| AA459544 | 2184451 |
| AA460383 | 2185596 |
| AA460941 | 2186061 |
| AA463563 | 2188447 |
| AA464471 | 2189355 |
| AA467869 | 2194403 |
| AA469319 | 2195853 |
| AA476568 | 2204779 |
| AA476980 | 2205191 |
| AA477822 | 2206456 |
| AA478382 | 2207016 |
| AA478397 | 2207031 |
| AA479044 | 2205407 |
| AA479490 | 2208046 |
| AA480144 | 2208295 |
| AA481078 | 2210630 |
| AA488519 | 2215950 |
| AA493269 | 2223110 |
| AA503327 | 2238294 |
| AA505810 | 2241947 |
| AA506026 | 2242163 |
| AA506542 | 2242297 |
| AA507244 | 2243683 |
| AA514617 | 2254217 |
| AA515132 | 2254732 |
| AA521134 | 2261677 |
| AA521377 | 2261920 |
| AA521478 | 2262021 |
| AA527168 | 2269237 |
| AA527704 | 2269773 |
| AA534504 | 2278757 |
| AA552146 | 2322398 |
| AA557336 | 2327813 |
| AA558876 | 2329643 |
| AA559209 | 2329405 |
| AA572791 | 2347319 |
| AA581467 | 2359239 |
| AA582612 | 2359972 |
| AA587269 | 2398083 |
| AA594137 | 2409487 |
| AA599533 | 2433158 |
| AA609167 | 2457595 |
| AA612898 | 2463936 |
| AA625833 | 2538220 |
| AA626477 | 2538864 |
| AA628322 | 2540709 |
| AA634277 | 2557491 |
| AA639123 | 2562902 |
| AA639223 | 2563002 |
| AA641277 | 2566527 |
| AA643063 | 2568281 |
| AA652845 | 2584497 |
| AA663986 | 2617977 |
| AA679329 | 2659851 |
| AA682527 | 2669808 |
| AA682861 | 2669544 |
| AA687499 | 2675690 |
| AA701625 | 2704790 |
| AA703094 | 2706207 |
| AA703179 | 2706292 |
| AA704155 | 2714073 |
| AA704856 | 2714774 |
| AA705590 | 2715508 |
| AA707255 | 2717173 |
| AA714838 | 2727112 |
| AA716568 | 2728842 |
| AA720598 | 2736733 |
| AA723130 | 2740837 |
| AA766044 | 2817282 |
| AA770043 | 2821281 |
| AA773324 | 2824895 |
| AA773727 | 2825298 |
| AA805504 | 2874254 |
| AA807426 | 2877002 |
| AA808186 | 2877592 |
| AA812594 | 2882658 |
| AA827097 | 2901094 |
| AA829474 | 2902573 |
| AA829769 | 2902868 |
| AA853584 | 2940323 |
| AA854927 | 2942465 |
| AA863276 | 2955755 |
| AA863446 | 2955925 |
| AA868174 | 2963619 |
| AA868640 | 2964085 |
| AA872126 | 2968304 |
| AA877795 | 2986760 |
| AA902103 | 3037293 |
| AA903253 | 3038376 |
| AA917448 | 3057338 |
| AA933679 | 3089947 |
| AA933684 | 3089952 |
| AA938929 | 3098842 |
| AA962515 | 3134679 |
| AA973392 | 3148572 |
| AA974390 | 3149570 |
| AA977809 | 3155255 |
| AA993601 | 3180146 |
| AF017688 | 2435451 |
| AF038251 | 2815881 |
| AI002420 | 3202754 |
| AI005164 | 3214674 |
| AI015844 | 3230180 |
| AI025782 | 3241395 |
| AI028404 | 3245713 |
| AI039096 | 3278290 |
| AI061159 | 3336527 |
| AI061422 | 3336790 |
| AI075189 | 3401780 |
| AI079233 | 3415484 |
| AI097371 | 3446953 |
| AI125755 | 3594269 |
| AI142257 | 3658616 |
| AI143987 | 3665796 |
| AI147744 | 3675426 |
| AI148558 | 3677027 |
| AI150088 | 3678557 |
| AI186525 | 3737163 |
| AI199904 | 3752510 |
| AI199936 | 3752542 |
| AI201570 | 3754176 |
| AI201576 | 3754182 |
| AI203647 | 3756253 |
| AI214250 | 3777851 |
| AI216862 | 3785903 |
| AI239435 | 3834832 |
| AI241369 | 3836766 |
| AI245345 | 3840742 |
| AI250290 | 3846819 |
| AI266663 | 3884821 |
| AI267185 | 3886352 |
| AI267425 | 3886592 |
| AI267612 | 3886779 |
| AI275090 | 3897364 |
| AI275233 | 3897507 |
| AI278769 | 3917003 |
| AI278776 | 3917010 |
| AI279562 | 3917796 |
| AI290088 | 3931754 |
| AI290518 | 3933292 |
| AI298973 | 3958627 |
| AI301926 | 3961272 |
| AI307771 | 4002375 |
| AI308800 | 4003671 |
| AI312603 | 4018208 |
| AI342411 | 4079338 |
| AI347826 | 4085032 |
| AI347995 | 4085201 |
| AI349645 | 4086851 |
| AI350809 | 4088015 |
| AI362793 | 4114414 |
| AI366549 | 4126238 |
| AI366974 | 4136719 |
| AI375141 | 4175131 |
| AI376613 | 4186466 |
| AI376640 | 4186493 |
| AI379614 | 4189467 |
| AI400282 | 4243369 |
| AI421827 | 4267758 |
| AI433157 | 4287209 |
| AI436202 | 4308535 |
| AI469427 | 4331517 |
| AI475758 | 4328803 |
| AI525199 | 4439334 |
| AI525579 | 4439714 |
| AI568908 | 4532282 |
| AI620381 | 4629507 |
| AI650799 | 4734778 |
| AI660145 | 4763715 |
| AI670903 | 4850634 |
| AI684386 | 4895680 |
| AI702062 | 4989962 |
| AI743061 | 5111349 |
| AL047763 | 5936402 |
| C03852 | 1467103 |
| C06289 | 1503065 |
| D54540 | 956437 |
| D58432 | 968066 |
| D82436 | 1183826 |
| F22086 | 2061262 |
| F27502 | 4813128 |
| H05168 | 868720 |
| H08641 | 873463 |
| H11991 | 876811 |
| H13883 | 878703 |
| H15247 | 880067 |
| H24046 | 892741 |
| H49462 | 989303 |
| H52955 | 993102 |
| H73244 | 1046589 |
| H84532 | 1063203 |
| H87703 | 1069282 |
| H95493 | 1108635 |
| N28826 | 1147062 |
| N34398 | 1155540 |
| N78296 | 1240997 |
| R45371 | 822227 |
| R55887 | 825993 |
| R59147 | 829842 |
| R63481 | 835360 |
| R73183 | 847215 |
| R76057 | 850739 |
| R88149 | 946962 |
| R91325 | 958865 |
| T24595 | 534220 |
| T29724 | 611822 |
| T30494 | 612592 |
| T35410 | 617508 |
| T84755 | 713107 |
| W05639 | 1278576 |
| W19700 | 1295599 |
| W24250 | 1301075 |
| W39447 | 1321173 |
| W45419 | 1329500 |
| W52961 | 1350395 |
| W60209 | 1366970 |
| Z21669 | 38542 |
| Z25220 | 395859 |
| Z45221 | 574427 |
| AB002368 | 2224680 |
| AB018315 | 3882264 |
| AB018346 | 3882326 |
| AF026548 | 2583172 |
| AF037439 | 2707343 |
| AF038650 | 4511880 |
| AF064491 | 3372806 |
| AF070598 | 3387976 |
| AF081280 | 3415120 |
| AF086336 | 3483681 |
| AF131826 | 4406662 |
| AL021683 | 3217033 |
| D00422 | 220063 |
| D26181 | 473712 |
| D84307 | 1817547 |
| D88532 | 1661000 |
| L40391 | 887356 |
| M28211 | 550067 |
| U01184 | 440176 |
| U14658 | 557469 |
| U40282 | 3150001 |
| U71598 | 1613851 |
| U94855 | 2055430 |
| Q32353 | notDefined |
| Q32355 | notDefined |
| Q46540 | notDefined |
| T47520 | 649500 |
| V59657 | notDefined |
| X19548 | notDefined |
| AI039871 | 3279065 |
| AA778432 | 2837763 |
| AI092886 | 3431862 |
| AA082314 | 1624370 |
| AA862355 | 2954834 |
| AI659859 | 4763429 |
| AL047713 | 4728709 |
| AI189911 | 3741120 |
| AA853117 | 2939856 |
| AA424894 | 2107331 |
| AI366549 | 4126238 |
| AA558957 | 2329724 |
| AA313534 | 1965864 |
| AI139211 | 3645183 |
| AI131084 | 3601100 |
| AA601051 | 2434676 |
| AI267615 | 3886782 |
| R71549 | 845066 |
| AI373754 | 4153620 |
| AA609618 | 2458046 |
| AA122401 | 1678777 |
| AA335862 | 1988104 |
| AI085616 | 3424039 |
| AI291072 | 3933846 |
| AA206225 | 1801795 |
| AI654989 | 4738968 |
| AA160532 | 1735917 |
| AA451923 | 2165592 |
| AA022750 | 1486823 |
| AA299694 | 1952025 |
| AA292009 | 1939986 |
| AA476770 | 2204981 |
| AA436859 | 2141773 |
| AA706420 | 2716338 |
| AA923755 | 3069021 |
| AA923755 | 3069021 |
| AA100982 | 1647709 |
| AA470067 | 2197376 |
| AA418433 | 2080262 |
| AA126150 | 1685875 |
| AA151805 | 1720500 |
| AA393176 | 2046164 |
| AA134909 | 1696071 |
| W29031 | 1308988 |
| AA482432 | 2210110 |
| AA115271 | 1670449 |
| AA219390 | 1833473 |
| AA232172 | 1855527 |
| AA159064 | 1733858 |
| AA358940 | 2011332 |
| AA041369 | 1517584 |
| AA063638 | 1557605 |
| AA233221 | 1856224 |
| AA609837 | 2458265 |
| AA130076 | 1690936 |
| AA307735 | 1960063 |
| AA063638 | 1557605 |
| AA032275 | 1502256 |
| AA437301 | 2142215 |
| AA977661 | 3155107 |
| AA977661 | 3155107 |
| AA436315 | 2141229 |
| AA453784 | 2167453 |
| AI282538 | 3920771 |
| AA335551 | 1987794 |
| AA335977 | 1988217 |
| Z44898 | 574083 |
| AA186404 | 1774704 |
| AA845426 | 2933185 |
| AA406233 | 2064375 |
| AA026625 | 1492460 |
| AA323696 | 1976024 |
| AI084071 | 3422494 |
| AI084071 | 3422494 |
| AA308585 | 1961065 |
| H96671 | 1110157 |
| AI082695 | 3419487 |
| AA215319 | 1815082 |
| AA047729 | 1527526 |
| AA437301 | 2142215 |
| AA081269 | 1623145 |
| AA148523 | 1721741 |
| AA148523 | 1721741 |
| AA054564 | 1545489 |
| AA037260 | 1512403 |
| U69188 | 2739412 |
| AA307058 | 1959536 |
| AA486182 | 2216398 |
| AI347167 | 4084373 |
| AA034975 | 1506919 |
| AA461052 | 2186172 |
| W31059 | 1312117 |
| AA576065 | 2350580 |
| AI346050 | 4083256 |
| Z44898 | 574083 |
| U69188 | 2739412 |
| W90000 | 1405978 |
| AI267379 | 3886546 |
| AA429418 | 2111938 |
| AA152275 | 1721678 |
| AA609837 | 2458265 |
| AA037260 | 1512403 |
| AA121249 | 1678863 |
| AA133375 | 1690361 |
| AA081204 | 1623261 |
| AA401204 | 2055157 |
| AA063074 | 1556627 |
| AI148825 | 3677294 |
| AA127645 | 1686969 |
| AA025006 | 1489911 |
| Z44898 | 574083 |
| AA194362 | 1784068 |
| AA515031 | 2254631 |
| AA429418 | 2111938 |
| AI148825 | 3677294 |
| W27631 | 1307597 |
| AA373030 | 2025349 |
| AI628638 | 4665438 |
| AA831467 | 2904566 |
| AA443231 | 2155906 |
| AA443231 | 2155906 |
| AI308800 | 4003671 |
| AA857509 | 2945811 |
| AA632557 | 2555971 |
| AA150653 | 1722165 |
| AA148885 | 1718994 |
| AA318422 | 1970750 |
| AI580104 | 4564480 |
| AA227945 | 1849525 |
| AA429418 | 2111938 |
| AA428362 | 2111876 |
| AA229611 | 1851608 |
| AI261664 | 3869867 |
| AA788933 | 2849053 |
| AA504389 | 2240549 |
| AA788933 | 2849053 |
| AA233122 | 1856134 |
| AA192168 | 1781991 |
| AA452930 | 2166599 |
| AA523928 | 2264856 |
| W27631 | 1307597 |
| R14684 | 768957 |
| AA452335 | 2166004 |
| AA291003 | 1938999 |
| AA443231 | 2155906 |
| AA443231 | 2155906 |
| AI241358 | 3836755 |
| AA070540 | 1577901 |
| AA581144 | 2358916 |
| AA581144 | 2358916 |
| AA928420 | 3077576 |
| AA291003 | 1938999 |
| AA227124 | 1848678 |
| AA969067 | 3144247 |
| AA760854 | 2809784 |
| AA580691 | 2358348 |
| AA770300 | 2821538 |
| AA770300 | 2821538 |
| AA083836 | 1625894 |
| AA045464 | 1523680 |
| AA167325 | 1745719 |
| AA115676 | 1670689 |
| AA187741 | 1773942 |
| AA352347 | 2004665 |
| AA375384 | 2027703 |
| AA136215 | 1697527 |
| AA318422 | 1970750 |
| AA244162 | 1874865 |
| AA211509 | 1810163 |
| AA430507 | 2111097 |
| AA515132 | 2254732 |
| AA284355 | 1928635 |
| AA167246 | 1745623 |
| AA083836 | 1625894 |
| AA113359 | 1665199 |
| AA669126 | 2630625 |
| AI683279 | 4893461 |
| AA296452 | 1948855 |
| AA765577 | 2816815 |
| AA086276 | 1628902 |
| AA214176 | 1812856 |
| AA476889 | 2205100 |
| AI016888 | 3231224 |
| AA600214 | 2433839 |
| AA375384 | 2027703 |
| AA453784 | 2167453 |
| AA453784 | 2167453 |
| W76487 | 1386732 |
| AA247690 | 1879591 |
| AA737182 | 2767457 |
| AA129463 | 1689334 |
| AA129463 | 1689334 |
| H04522 | 867455 |
| AA169457 | 1748396 |
| AA010849 | 1471875 |
| AA156616 | 1728239 |
| AA431321 | 2115029 |
| AA558281 | 2328758 |
| AA581144 | 2358916 |
| AA935526 | 3092683 |
| AA088373 | 1633885 |
| AA453445 | 2167114 |
| AA968726 | 3143906 |
| AA770537 | 2821775 |
| AA330007 | 1982249 |
| AA330007 | 1982249 |
| AA853539 | 2940278 |
| T30659 | 612757 |
| AA501373 | 2236340 |
| AA935526 | 3092683 |
| AI672524 | 4852255 |
| AI274797 | 3897071 |
| AI129827 | 3598341 |
| AI478372 | 4371598 |
| AA665560 | 2620173 |
| AA788933 | 2849053 |
| AA161518 | 1735828 |
| AA041467 | 1517765 |
| AI074333 | 3400977 |
| AA044787 | 1523055 |
| AA825917 | 2899229 |
| AA825917 | 2899229 |
| F27264 | 4812890 |
| H97360 | 1118229 |
| AA313688 | 1966017 |
| AA024982 | 1489887 |
| AI693441 | 4970781 |
| AI351167 | 4088373 |
| AA398886 | 2051923 |
| AA621740 | 2524168 |
| AI539679 | 4453814 |
| AA426083 | 2107376 |
| AA399022 | 2052821 |
| AA412184 | 2070755 |
| AA788933 | 2849053 |
| AA376374 | 2028692 |
| AI690181 | 4901475 |
| AA213902 | 1812647 |
| AA514564 | 2254164 |
| AA701870 | 2704983 |
| AA486954 | 2217118 |
| AA284462 | 1928769 |
| AA465528 | 2191695 |
| AA171834 | 1750944 |
| AI081515 | 3418307 |
| AI267532 | 3886699 |
| AL048644 | 4727432 |
| AA281739 | 1924484 |
| AA570181 | 2344161 |
| AI660919 | 4764502 |
| AA088764 | 1634285 |
| AA903285 | 3038408 |
| AA283080 | 1926005 |
| AA465285 | 2191452 |
| AA159916 | 1734424 |
| AA026215 | 1492901 |
| AA043908 | 1521766 |
| AA375325 | 2027655 |
| AI052317 | 3308308 |
| AA194535 | 1784231 |
| AA846439 | 2932579 |
| AA492032 | 2221594 |
| AI026767 | 3246255 |
| AI004557 | 3214067 |
| AA335502 | 1987807 |
| AA001794 | 1445608 |
| AA333713 | 1985957 |
| AA730829 | 2752033 |
| AA127626 | 1686979 |
| AA101212 | 1647866 |
| AA303085 | 1955415 |
| AI673189 | 4852920 |
| AI086254 | 3424677 |
| AA035773 | 1507601 |
| AA916110 | 3055502 |
| AA166874 | 1745038 |
| AA422177 | 2101028 |
| AA262878 | 1898599 |
| AA282134 | 1925013 |
| AA156237 | 1727855 |
| AA425378 | 2106152 |
| AA150409 | 1721922 |
| AA045417 | 1523619 |
| AI052317 | 3308308 |
| W01842 | 1273822 |
| AA425292 | 2106102 |
| AA985622 | 3167011 |
| AA722855 | 2740562 |
| AA426216 | 2107619 |
| AA788933 | 2849053 |
| AI138898 | 3644870 |
| AA778250 | 2837581 |
| W78198 | 1388230 |
| AA788933 | 2849053 |
| AI342607 | 4079534 |
| AA426216 | 2107619 |
| AA927120 | 3076017 |
| AA788933 | 2849053 |
| AI079871 | 3416122 |
| AI741829 | 5110117 |
| AA418715 | 2080579 |
| AA150636 | 1722148 |
| AI433965 | 4292869 |
| AA088373 | 1633885 |
| AA471070 | 2198379 |
| AI673422 | 4853153 |
| AA203123 | 1798926 |
| AA203627 | 1799397 |
| AA193592 | 1782993 |
| AA206064 | 1801436 |
| R31249 | 787092 |
| AA788933 | 2849053 |
| AA282369 | 1925321 |
| AA149594 | 1720413 |
| AA347635 | 1999892 |
| AA293172 | 1941195 |
| AA832167 | 2905266 |
| AA053133 | 1544273 |
| AA908731 | 3048136 |
| AA808830 | 2878236 |
| AA779747 | 2839078 |
| AA084038 | 1626094 |
| AI365500 | 4125189 |
| AI609252 | 4618419 |
| H08650 | 873472 |
| H92511 | 1088089 |
| AA088373 | 1633885 |
| H66289 | 1025029 |
| W73086 | 1383220 |
| AA035773 | 1507601 |
| AA481432 | 2210984 |
| AA082084 | 1624176 |
| AA490665 | 2219838 |
| AA429541 | 2112641 |
| AA568231 | 2341285 |
| AA426216 | 2107619 |
| AA738430 | 2769187 |
| AA627132 | 2540176 |
| AA402714 | 2056470 |
| AA025585 | 1491013 |
| AA493305 | 2223146 |
| AA278542 | 1919927 |
| AA278542 | 1919927 |
| AA442936 | 2155611 |
| AA779747 | 2839078 |
| AI283155 | 3921388 |
| AI263446 | 3871649 |
| AA013042 | 1474088 |
| AA081426 | 1623343 |
| AA484061 | 2212874 |
| AA315535 | 1967884 |
| AA399022 | 2052821 |
| AI127825 | 3596339 |
| AA715828 | 2728102 |
| AI243931 | 3839328 |
| AI024753 | 3240366 |
| AA086070 | 1629620 |
| AA625159 | 2537544 |
| AA426216 | 2107619 |
| AA426216 | 2107619 |
| AI628734 | 4665534 |
| AI358846 | 4110467 |
| AA013042 | 1474088 |
| AI273856 | 3896124 |
| AA493647 | 2223488 |
| AA450075 | 2163825 |
| AA115777 | 1670800 |
| AA494342 | 2224129 |
| AA181331 | 1764814 |
| AA418715 | 2080579 |
| AA662648 | 2616639 |
| AA086070 | 1629620 |
| AA487250 | 2217414 |
| AA099101 | 1644961 |
| AA093957 | 1639542 |
| AA136215 | 1697527 |
| AA256459 | 1891997 |
| AI760421 | 5176088 |
| AA082675 | 1624733 |
| AA180746 | 1764253 |
| AA534365 | 2278618 |
| W28567 | 1308722 |
| AA069657 | 1577016 |
| AA178880 | 1760241 |
| H23090 | 891785 |
| AA130285 | 1691429 |
| AI052317 | 3308308 |
| AA453784 | 2167453 |
| AA312917 | 1965244 |
| AA521024 | 2261567 |
| AA188744 | 1775770 |
| AA593011 | 2408773 |
| N78223 | 1240924 |
| AA669106 | 2630605 |
| AA548445 | 2318727 |
| AA046827 | 1524726 |
| AA618310 | 2505515 |
| AA426216 | 2107619 |
| AA281412 | 1924091 |
| AA532835 | 2278411 |
| AA809070 | 2878476 |
| AI608739 | 4617906 |
| AA977802 | 3155248 |
| H68725 | 1030654 |
| AA125809 | 1688020 |
| AI299874 | 3957782 |
| AA453784 | 2167453 |
| AA101236 | 1647876 |
| AA491219 | 2220392 |
| AA149853 | 1720933 |
| AA151345 | 1719890 |
| AI273856 | 3896124 |
| AA405425 | 2063662 |
| AA599864 | 2433489 |
| AA411436 | 2068968 |
| AA131439 | 1692926 |
| AA706347 | 2716265 |
| AI349772 | 4086978 |
| AA488463 | 2215894 |
| AA083482 | 1625543 |
| AA083482 | 1625543 |
| AA149853 | 1720933 |
| AA255613 | 1892460 |
| AA627448 | 2539543 |
| AA453784 | 2167453 |
| AA022788 | 1486887 |
| AA635003 | 2558217 |
| AA576419 | 2353919 |
| AA459880 | 2184787 |
| AA131053 | 1692543 |
| AA496640 | 2229961 |
| AI582629 | 4568526 |
| AI582629 | 4568526 |
| AI023912 | 3238956 |
| AA977802 | 3155248 |
| W00823 | 1272803 |
| W00823 | 1272803 |
| AI674397 | 4874877 |
| AA700350 | 2703313 |
| AA745614 | 2785600 |
| AI302190 | 3961536 |
| AA393518 | 2046486 |
| N27985 | 1142466 |
| AA468753 | 2195287 |
| AA653775 | 2589929 |
| AA653775 | 2589929 |
| AA088373 | 1633885 |
| AA088373 | 1633885 |
| AA130783 | 1692449 |
| AA461143 | 2186263 |
| R78778 | 855059 |
| AA031550 | 1501548 |
| AA461143 | 2186263 |
| AA303484 | 1955818 |
| AA301297 | 1953649 |
| AA744503 | 2783267 |
| T67813 | 678961 |
| AI564319 | 4522776 |
| AI564319 | 4522776 |
| AI138898 | 3644870 |
| AI138898 | 3644870 |
| AA301297 | 1953649 |
| AI302436 | 3961782 |
| H27251 | 897241 |
| AA418715 | 2080579 |
| C01749 | 1433979 |
| AA083471 | 1625557 |
| AI264186 | 3872389 |
| T66196 | 675241 |
| H29445 | 900355 |
| AI469093 | 4331183 |
| AA136215 | 1697527 |
| AI267185 | 3886352 |
| AA935526 | 3092683 |
| AA026157 | 1492760 |
| AA001277 | 1437430 |
| AA552588 | 2322842 |
| AA099921 | 1646060 |
| AA380495 | 2032813 |
| R77384 | 852016 |
| AA024924 | 1489830 |
| AI309007 | 4003878 |
| AA278434 | 1919771 |
| AA303150 | 1955481 |
| AA460060 | 2184944 |
| AA169355 | 1748429 |
| AI201573 | 3754179 |
| AA779747 | 2839078 |
| AA468753 | 2195287 |
| AA091431 | 1636886 |
| AA464646 | 2189530 |
| AA083836 | 1625894 |
| AA151506 | 1720046 |
| AA069195 | 1576616 |
| AA007137 | 1463255 |
| AA178912 | 1760460 |
| AA225164 | 1846568 |
| AI565125 | 4523582 |
| AA483181 | 2211994 |
| AA470909 | 2198218 |
| AA650493 | 2577821 |
| AA059309 | 1552153 |
| AA677142 | 2657664 |
| AA447503 | 2161173 |
| AA252216 | 1887179 |
| F33257 | 4818883 |
| AA576667 | 2354141 |
| AA320124 | 1972473 |
| AA082569 | 1624827 |
| AA573200 | 2347728 |
| AA178880 | 1760241 |
| AA176433 | 1757557 |
| AA788933 | 2849053 |
| AI033037 | 3253990 |
| AA533283 | 2277379 |
| AI267185 | 3886352 |
| AA280123 | 1921984 |
| C17183 | 1571890 |
| AA779747 | 2839078 |
| AI283155 | 3921388 |
| AA308736 | 1961086 |
| H52011 | 991852 |
| AA034975 | 1506919 |
| AA034908 | 1506871 |
| AA564296 | 2335935 |
| AA515031 | 2254631 |
| AA083471 | 1625557 |
| AA088373 | 1633885 |
| AA088373 | 1633885 |
| AI218204 | 3798019 |
| N46402 | 1187568 |
| AA099023 | 1644903 |
| AA600987 | 2434612 |
| AI267185 | 3886352 |
| N46402 | 1187568 |
| N46402 | 1187568 |
| AA131053 | 1692543 |
| R19004 | 772614 |
| AA099878 | 1645960 |
| AI608720 | 4617887 |
| AI131214 | 3601230 |
| AI139097 | 3645069 |
| AA089857 | 1636357 |
| AA047849 | 1527546 |
| AA058822 | 1551648 |
| AA147978 | 1717352 |
| AA199850 | 1795557 |
| AI167659 | 3700829 |
| AI167659 | 3700829 |
| AA574054 | 2348569 |
| W37586 | 1319191 |
| AA088373 | 1633885 |
| AA157619 | 1729227 |
| AA013042 | 1474088 |
| AA300170 | 1952574 |
| AA430088 | 2113325 |
| AA136096 | 1697324 |
| AA136756 | 1697984 |
| W07393 | 1281394 |
| AA143577 | 1712949 |
| AA642766 | 2567984 |
| AA554920 | 2325459 |
| AA452335 | 2166004 |
| AA532484 | 2276738 |
| AA609837 | 2458265 |
| AA152275 | 1721678 |
| AI192851 | 3744060 |
| AA228082 | 1849644 |
| AI093501 | 3432477 |
| AA905277 | 3040400 |
| AA465301 | 2191468 |
| AA172248 | 1751307 |
| AA129819 | 1689431 |
| AA428120 | 2111789 |
| AI291105 | 3933879 |
| AI750962 | 5129226 |
| AA011024 | 1472051 |
| AA873159 | 2969281 |
| AA873159 | 2969281 |
| AA846266 | 2932406 |
| AA652080 | 2583732 |
| AI339481 | 4076408 |
| AA609172 | 2457600 |
| AA609172 | 2457600 |
| AA908731 | 3048136 |
| AI147200 | 3674882 |
| AA864497 | 2958810 |
| AA083482 | 1625543 |
| AI279718 | 3917952 |
| AA188375 | 1775409 |
| AA088373 | 1633885 |
| AA280110 | 1921971 |
| AA744712 | 2783476 |
| AA936089 | 3093246 |
| AA076368 | 1616254 |
| AA774663 | 2833997 |
| AA527389 | 2269458 |
| AA007495 | 1463481 |
| AA045264 | 1523468 |
| AA523252 | 2263964 |
| AA552588 | 2322842 |
| AA173924 | 1754056 |
| AA465301 | 2191468 |
| AA599533 | 2433158 |
| AA614105 | 2466239 |
| AA384078 | 2036501 |
| AA872507 | 2968685 |
| AA045936 | 1525830 |
| AA081269 | 1623145 |
| AA572758 | 2358298 |
| AA007271 | 1463305 |
| Z78349 | 1495122 |
| AA626469 | 2538856 |
| AA551215 | 2321467 |
| AA621740 | 2524168 |
| AA304249 | 1956602 |
| AA877288 | 2986365 |
| AA483182 | 2211995 |
| N78223 | 1240924 |
| N78268 | 1240969 |
| AA365398 | 2017716 |
| AA131231 | 1692739 |
| R55380 | 824675 |
| AI078529 | 3412937 |
| AI767123 | 5233632 |
| AI700226 | 4988126 |
| AA487586 | 2217750 |
| AI033304 | 3254257 |
| AA436895 | 2141809 |
| W01842 | 1273822 |
| AA864853 | 2959166 |
| AA444134 | 2156809 |
| AI700226 | 4988126 |
| AA853130 | 2939869 |
| H52011 | 991852 |
| AA618484 | 2505689 |
| AA101484 | 1648365 |
| AA649746 | 2577074 |
| H01401 | 864334 |
| AA375933 | 2028282 |
| AA166905 | 1745299 |
| AA151916 | 1720789 |
| AA845573 | 2933332 |
| AA013363 | 1474399 |
| AI096880 | 3446462 |
| AA905781 | 3040904 |
| AI052317 | 3308308 |
| AA513827 | 2252248 |
| AI393144 | 4222691 |
| AA788933 | 2849053 |
| AA788933 | 2849053 |
| AA311098 | 1963425 |
| AA542829 | 2291309 |
| AA166622 | 1745211 |
| AI054163 | 3321950 |
| AA164243 | 1741190 |
| AI288783 | 3932286 |
| AI186605 | 3737243 |
| C18682 | 1580284 |
| AA340224 | 1992461 |
| AA171834 | 1750944 |
| AA196793 | 1792366 |
| AI423079 | 4269010 |
| AI423079 | 4269010 |
| AI521128 | 4435263 |
| AA526226 | 2268295 |
| AA165306 | 1740534 |
| AA878771 | 2987736 |
| AA195865 | 1791530 |
| W16508 | 1291096 |
| AA995597 | 3182086 |
| AI042002 | 3281196 |
| AA936566 | 3094484 |
| AA936566 | 3094484 |
| AA977802 | 3155248 |
| AA913590 | 3052982 |
| AA913590 | 3052982 |
| AI360219 | 4111840 |
| AA804436 | 2875949 |
| AA548082 | 2318364 |
| T66196 | 675241 |
| AA199802 | 1795511 |
| AI040294 | 3279488 |
| AA402901 | 2056688 |
| AA426653 | 2107306 |
| AA001794 | 1445608 |
| AA514564 | 2254164 |
| D54004 | 955901 |
| AA211272 | 1809917 |
| AA161003 | 1735290 |
| AA232705 | 1855905 |
| AA020855 | 1484716 |
| AA081395 | 1623312 |
| AA885514 | 2994591 |
| T71404 | 685925 |
| W01842 | 1273822 |
| AA094609 | 1640194 |
| AA062957 | 1557458 |
| AA026215 | 1492901 |
| AA158724 | 1733518 |
| AI243555 | 3838952 |
| AA026719 | 1492500 |
| AA036944 | 1510001 |
| AA076262 | 1616131 |
| AA120908 | 1678331 |
| AA161066 | 1735498 |
| AI391479 | 4217483 |
| AA343324 | 1995582 |
| N77582 | 1240283 |
| AA083471 | 1625557 |
| AA872507 | 2968685 |
| AI093740 | 3432716 |
| AA731306 | 2752510 |
| AA305494 | 1958072 |
| AA699927 | 2702890 |
| AA701448 | 2704613 |
| AI554245 | 4486608 |
| AA132605 | 1694094 |
| AA825917 | 2899229 |
| W78198 | 1388230 |
| AI609252 | 4618419 |
| AA022788 | 1486887 |
| AA026625 | 1492460 |
| AA206519 | 1801900 |
| AA256335 | 1891872 |
| AA256335 | 1891872 |
| AA013363 | 1474399 |
| AI300590 | 3959936 |
| AA069733 | 1577093 |
| AA182841 | 1766558 |
| AI342480 | 4079407 |
| AA451894 | 2165563 |
| AA915959 | 3055351 |
| AA147833 | 1717205 |
| AA229993 | 1852287 |
| AA199821 | 1795609 |
| AI198431 | 3751037 |
| AA928420 | 3077576 |
| AA256320 | 1891857 |
| AI267185 | 3886352 |
| AA528456 | 2270525 |
| AI567965 | 4526417 |
| AA101299 | 1648045 |
| AA618484 | 2505689 |
| AI278872 | 3917106 |
| AI278872 | 3917106 |
| AA708712 | 2718630 |
| AA398892 | 2051929 |
| N52168 | 1193302 |
| AA827758 | 2901317 |
| R56747 | 826853 |
| AA854014 | 2941552 |
| AA904266 | 3039389 |
| AA770557 | 2821795 |
| AL048446 | 5936506 |
| AA062817 | 1557177 |
| AA464646 | 2189530 |
| AA323097 | 1975422 |
| AA102168 | 1646226 |
| AA528121 | 2270190 |
| AA526498 | 2268567 |
| AA877213 | 2986290 |
| AA864690 | 2959003 |
| AA864690 | 2959003 |
| AA788933 | 2849053 |
| AA235835 | 1860273 |
| AA558066 | 2328543 |
| AA149151 | 1719587 |
| AA583526 | 2368135 |
| AA013363 | 1474399 |
| AA013363 | 1474399 |
| AI141567 | 3649024 |
| AA187406 | 1773598 |
| AI499986 | 4391968 |
| AA488398 | 2215829 |
| AA418408 | 2080217 |
| AA418408 | 2080217 |
| AA147312 | 1716726 |
| AA581130 | 2358902 |
| AA582851 | 2360211 |
| AA075393 | 1615264 |
| AA737562 | 2768319 |
| AI538172 | 4452307 |
| N57174 | 1201064 |
| AA453479 | 2167148 |
| AA442561 | 2154439 |
| AA448973 | 2162993 |
| AI079871 | 3416122 |
| AA487467 | 2217631 |
| AA442948 | 2155623 |
| AA078387 | 1837861 |
| AA620980 | 2524919 |
| AA195128 | 1784839 |
| AI126689 | 3595203 |
| AA446578 | 2159243 |
| AA649064 | 2575493 |
| AA938757 | 3096785 |
| AA806532 | 2875282 |
| AA316566 | 1968914 |
| H98646 | 1123314 |
| AA635393 | 2559235 |
| AA191045 | 1779655 |
| AI198431 | 3751037 |
| AA595562 | 2410912 |
| AA532383 | 2276637 |
| AA430059 | 2113233 |
| AA609837 | 2458265 |
| AA126418 | 1686030 |
| AI083558 | 3421981 |
| AA465584 | 2191751 |
| AI129865 | 3598379 |
| AA316089 | 1968579 |
| AA341011 | 1993280 |
| W60762 | 1367521 |
| R24022 | 778910 |
| R25322 | 781457 |
| AI249905 | 3846434 |
| AA126798 | 1686280 |
| AA551607 | 2321859 |
| AA418783 | 2080593 |
| AA151600 | 1720087 |
| AA600173 | 2433798 |
| AI245990 | 3841387 |
| AI056158 | 3330024 |
| AA009816 | 1470796 |
| R91618 | 959158 |
| AA977802 | 3155248 |
| AI741118 | 5109406 |
| AA039810 | 1516299 |
| AA357956 | 2010500 |
| AA431278 | 2114986 |
| AI096925 | 3446507 |
| AA400833 | 2054705 |
| AI309380 | 4004251 |
| AA330640 | 1982904 |
| AA169457 | 1748396 |
| AI079871 | 3416122 |
| AA236339 | 1858389 |
| AI301952 | 3961298 |
| W03618 | 1275465 |
| W03618 | 1275465 |
| AA543080 | 2291560 |
| AA303085 | 1955415 |
| AA779747 | 2839078 |
| AA127826 | 1687133 |
| AA364229 | 2016548 |
| AA364229 | 2016548 |
| AA747475 | 2787433 |
| F20791 | 2059967 |
| F20791 | 2059967 |
| AA814278 | 2883874 |
| N39195 | 1162402 |
| AI700982 | 4988882 |
| AA136215 | 1697527 |
| AI089782 | 3428841 |
| AA827570 | 2900011 |
| AA983883 | 3162408 |
| AA192168 | 1781991 |
| AI097410 | 3446992 |
| AA742474 | 2782056 |
| W69615 | 1378874 |
| AI051843 | 3307377 |
| R60403 | 831098 |
| AA101010 | 1647531 |
| AA932039 | 3087081 |
| AA496669 | 2229990 |
| AA283707 | 1927919 |
| AA088344 | 1633856 |
| AA654827 | 2590981 |
| AI246125 | 3841522 |
| AI079871 | 3416122 |
| AI079871 | 3416122 |
| N88286 | 1441488 |
| AI738508 | 5100489 |
| AA070469 | 1577829 |
| AA256335 | 1891872 |
| AA256335 | 1891872 |
| AA010044 | 1471123 |
| AA091053 | 1635637 |
| AA000981 | 1437188 |
| AA148023 | 1717447 |
| AA622574 | 2526450 |
| AA936960 | 3094581 |
| AI201573 | 3754179 |
| AI344021 | 4081227 |
| AI127179 | 3595693 |
| AA026454 | 1492354 |
| AA026454 | 1492354 |
| AA136756 | 1697984 |
| AA129410 | 1689296 |
| AI032188 | 3250400 |
| AI032188 | 3250400 |
| AI741829 | 5110117 |
| AI214206 | 3777807 |
| AA314222 | 1966571 |
| AA099592 | 1645605 |
| AA765228 | 2816466 |
| AA831729 | 2904828 |
| AA190559 | 1779534 |
| W01842 | 1273822 |
| N31402 | 1151801 |
| AI267185 | 3886352 |
| AA492465 | 2222027 |
| AI611173 | 4620340 |
| AI311440 | 4006311 |
| AA447503 | 2161173 |
| AA412359 | 2070929 |
| AA700540 | 2703503 |
| AI332755 | 4069314 |
| AA115271 | 1670449 |
| AA009997 | 1471025 |
| AA150445 | 1721958 |
| AA741015 | 2779607 |
| AA397741 | 2050632 |
| AA723209 | 2740846 |
| AA773607 | 2825178 |
| AI309007 | 4003878 |
| AA977584 | 3155030 |
| AA143311 | 1712682 |
| W95589 | 1425517 |
| AA514638 | 2254238 |
| AI267148 | 3886315 |
| AI311440 | 4006311 |
| AI621272 | 4630398 |
| AI559470 | 4509675 |
| AA325222 | 1977466 |
| AA325222 | 1977466 |
| AI369447 | 4148200 |
| AA773804 | 2825375 |
| AA490210 | 2219392 |
| AA253384 | 1885689 |
| AA652436 | 2584088 |
| AA983883 | 3162408 |
| AA323560 | 1976131 |
| R21821 | 776602 |
| AA182841 | 1766558 |
| AA255567 | 1892326 |
| AA610706 | 2459134 |
| AA306015 | 1958487 |
| AA565996 | 2337635 |
| AI049770 | 3298887 |
| AA199644 | 1795527 |
| AA224371 | 1844949 |
| AA913590 | 3052982 |
| AA523526 | 2264238 |
| AI144108 | 3665917 |
| AA377383 | 2029964 |
| AA436438 | 2141352 |
| AA788933 | 2849053 |
| AI025517 | 3241130 |
| AA447250 | 2159915 |
| AA722512 | 2740219 |
| AA447590 | 2161260 |
| AA155915 | 1727533 |
| AA412059 | 2070648 |
| AA375325 | 2027655 |
| AA082684 | 1624742 |
| N47128 | 1188294 |
| AA227610 | 1849172 |
| R85339 | 943745 |
| R85339 | 943745 |
| AA521024 | 2261567 |
| AA069611 | 1577032 |
| AA303084 | 1955414 |
| AA426216 | 2107619 |
| AA706730 | 2716648 |
| R24799 | 779687 |
| AA129744 | 1690154 |
| AA436541 | 2141455 |
| AA524721 | 2265649 |
| AI492230 | 4393233 |
| AA209340 | 1807354 |
| AA974657 | 3149837 |
| AA281412 | 1924091 |
| AA368906 | 2021224 |
| AA101820 | 1644916 |
| AA302450 | 1954781 |
| N49477 | 1190643 |
| AA399022 | 2052821 |
| AI453471 | 4282174 |
| AA699666 | 2703813 |
| AA372894 | 2025234 |
| AI217001 | 3789655 |
| AA328393 | 1980637 |
| AI249705 | 3846234 |
| AI090943 | 3430002 |
| AA126836 | 1686318 |
| AI536912 | 4451047 |
| AA935134 | 3092291 |
| AA994818 | 3181307 |
| AI342714 | 4079920 |
| AA916068 | 3055460 |
| AA375384 | 2027703 |
| AA310694 | 1963043 |
| AA037823 | 1512941 |
| AA190559 | 1779534 |
| AA332853 | 1985159 |
| AA827899 | 2900262 |
| AA065319 | 1929234 |
| AA573238 | 2347766 |
| AA573238 | 2347766 |
| N99772 | 1271333 |
| AA192253 | 1782078 |
| AI017852 | 3232188 |
| AA203140 | 1798998 |
| AA345827 | 1998106 |
| AI129468 | 3597982 |
| AA151675 | 1720230 |
| AA157619 | 1729227 |
| AI204088 | 3756694 |
| AA232691 | 1855703 |
| AA232691 | 1855703 |
| AI267454 | 3886621 |
| AA464646 | 2189530 |
| AA496961 | 2230282 |
| AA496961 | 2230282 |
| AA779747 | 2839078 |
| AI433157 | 4287209 |
| AA464646 | 2189530 |
| AI311440 | 4006311 |
| AI127835 | 3596349 |
| AA279553 | 1921035 |
| AA227876 | 1849429 |
| AA455007 | 2177783 |
| AA169457 | 1748396 |
| AA169457 | 1748396 |
| AA766864 | 2818102 |
| AA026454 | 1492354 |
| AA026454 | 1492354 |
| AI074333 | 3400977 |
| AI074333 | 3400977 |
| AA448332 | 2162002 |
| F27264 | 4812890 |
| AA188481 | 1775524 |
| AI738508 | 5100489 |
| AI738508 | 5100489 |
| AA928420 | 3077576 |
| AI742056 | 5110344 |
| AA489412 | 2219014 |
| AI066421 | 3367123 |
| AA368063 | 2020380 |
| Z45013 | 574202 |
| AA490731 | 2219904 |
| AA316071 | 1968400 |
| AA872059 | 2968237 |
| AA083836 | 1625894 |
| AA199821 | 1795609 |
| AA187824 | 1774152 |
| AA913590 | 3052982 |
| AA913590 | 3052982 |
| AI074572 | 3401216 |
| AA101077 | 1647597 |
| AA668553 | 2630052 |
| AA165471 | 1741505 |
| AA286751 | 1933614 |
| AI440495 | 4300628 |
| AA526893 | 2268962 |
| AI278160 | 3900428 |
| AI242593 | 3837990 |
| AA622574 | 2526450 |
| AI591008 | 4600056 |
| AA365398 | 2017716 |
| AA173885 | 1754160 |
| T08682 | 389710 |
| AA074869 | 1614765 |
| AA357299 | 2009701 |
| AA203140 | 1798998 |
| AA190559 | 1779534 |
| AI168018 | 3701188 |
| AA451894 | 2165563 |
| AA024859 | 1489773 |
| AA412184 | 2070755 |
| AA207216 | 1802837 |
| AA463446 | 2188330 |
| AA135572 | 1696603 |
| AI364978 | 4124667 |
| AI281972 | 3920205 |
| AA418408 | 2080217 |
| AA418408 | 2080217 |
| AA074098 | 1613985 |
| AA029567 | 1497035 |
| AA747547 | 2787505 |
| AI753431 | 5131695 |
| AA232691 | 1855703 |
| AI079871 | 3416122 |
| AI300541 | 3959887 |
| AA417006 | 2077194 |
| AA490731 | 2219904 |
| Z45013 | 574202 |
| AI066421 | 3367123 |
| AA621714 | 2525653 |
| AA813524 | 2882209 |
| AA515261 | 2254861 |
| AA040300 | 1516745 |
| AA040300 | 1516745 |
| AI241763 | 3837160 |
| AA722512 | 2740219 |
| AI034147 | 3255100 |
| AI703111 | 4991011 |
| AI079871 | 3416122 |
| AI079871 | 3416122 |
| AA485781 | 2215000 |
| AA216667 | 1817393 |
| AA216667 | 1817393 |
| AI267282 | 3886449 |
| AA485488 | 2214707 |
| AA295804 | 1948159 |
| AA601147 | 2434772 |
| H06792 | 870324 |
| AA283707 | 1927919 |
| AA101077 | 1647597 |
| AI076532 | 3405710 |
| AI076532 | 3405710 |
| AA609534 | 2457962 |
| AA232691 | 1855703 |
| R60403 | 831098 |
| AA928676 | 3076967 |
| AA807960 | 2877366 |
| AA573742 | 2348257 |
| AA463547 | 2188431 |
| AI086042 | 3424465 |
| AI017852 | 3232188 |
| AI267185 | 3886352 |
| AA167794 | 1745006 |
| AI253335 | 3850456 |
| AA005232 | 1448694 |
| AA581144 | 2358916 |
| AI204127 | 3756733 |
| AA420989 | 2099822 |
| AA877877 | 2986842 |
| AA364275 | 2016676 |
| AA455007 | 2177783 |
| AA091539 | 1637002 |
| AA088373 | 1633885 |
| AA088373 | 1633885 |
| AA595217 | 2410567 |
| AA642691 | 2567909 |
| AI417520 | 4261024 |
| AA133375 | 1690361 |
| AI472536 | 4325581 |
| AA772548 | 2824331 |
| AA772548 | 2824331 |
| AA161488 | 1735798 |
| AA609253 | 2457681 |
| AA412184 | 2070755 |
| AA679015 | 2659537 |
| AA280235 | 1921773 |
| AI080011 | 3416262 |
| AI091013 | 3430072 |
| AA905362 | 3040485 |
| AA521069 | 2261612 |
| AA226674 | 1847990 |
| AA226674 | 1847990 |
| AA037506 | 1512624 |
| AA522790 | 2263502 |
| AA375384 | 2027703 |
| AA788933 | 2849053 |
| AI418922 | 4264853 |
| AA420989 | 2099822 |
| AA323097 | 1975422 |
| AI432949 | 4285754 |
| AI432949 | 4285754 |
| AA226914 | 1848450 |
| AA442097 | 2153975 |
| AA316566 | 1968914 |
| H98646 | 1123314 |
| AI281417 | 3919650 |
| AA283707 | 1927919 |
| AA505080 | 2241240 |
| AA505080 | 2241240 |
| AA789020 | 2849140 |
| AA789020 | 2849140 |
| N30234 | 1148754 |
| N98658 | 1270080 |
| AA323560 | 1976131 |
| AA719473 | 2732572 |
| AA446943 | 2159608 |
| AA419395 | 2079122 |
| AA419395 | 2079122 |
| AI718403 | 5035659 |
| AA115025 | 1670278 |
| AA789013 | 2849133 |
| AA830249 | 2903348 |
| AA830249 | 2903348 |
| AA282848 | 1925889 |
| AA136215 | 1697527 |
| AA336515 | 1988961 |
| AA854982 | 2942520 |
| AA533727 | 2277743 |
| AA121207 | 1678846 |
| AA424541 | 2103511 |
| AA040300 | 1516745 |
| AA923750 | 3069016 |
| AA173885 | 1754160 |
| AA352308 | 2004648 |
| AA233105 | 1856099 |
| H71437 | 1043253 |
| AI076532 | 3405710 |
| AA650333 | 2577661 |
| AA702428 | 2705541 |
| AA531613 | 2274319 |
| AA255584 | 1892556 |
| AA255584 | 1892556 |
| AI141699 | 3649156 |
| AA481432 | 2210984 |
| AA305599 | 1957924 |
| H10703 | 875554 |
| AA121879 | 1679511 |
| AI249797 | 3846326 |
| AA482853 | 2211698 |
| AI141567 | 3649024 |
| H01413 | 864346 |
| AA148508 | 1721552 |
| AA729727 | 2751086 |
| AA789158 | 2849278 |
| AA679848 | 2656315 |
| AA679848 | 2656315 |
| AI221967 | 3804170 |
| AA838613 | 2913412 |
| AI223887 | 3806600 |
| W57570 | 1364294 |
| H23090 | 891785 |
| AI203342 | 3755948 |
| AA056334 | 1548691 |
| AI088115 | 3427091 |
| AA373243 | 2025583 |
| AI359989 | 4111610 |
| AA247180 | 1878685 |
| AA838613 | 2913412 |
| AA136215 | 1697527 |
| AA876375 | 2985452 |
| AA876375 | 2985452 |
| AA905495 | 3040618 |
| AI344928 | 4082134 |
| AA769315 | 2820553 |
| F37855 | 4837254 |
| AI093165 | 3432141 |
| AA478787 | 2207421 |
| AA088344 | 1633856 |
| AA766044 | 2817282 |
| AI339481 | 4076408 |
| AA829473 | 2902572 |
| D20248 | 501345 |
| AA412015 | 2070586 |
| AA420989 | 2099822 |
| AA259065 | 1894545 |
| AI345847 | 4083053 |
| AA235453 | 1859908 |
| AA486335 | 2215141 |
| AI129482 | 3597996 |
| AI690374 | 4901676 |
| AI034481 | 3253412 |
| AI312562 | 4018167 |
| AI312562 | 4018167 |
| AI092921 | 3431897 |
| AA570668 | 2344648 |
| AA489032 | 2218634 |
| AA047729 | 1527526 |
| AA662918 | 2616909 |
| AI078151 | 3412559 |
| AA233672 | 1856665 |
| AA157424 | 1729032 |
| AI078151 | 3412559 |
| AA043258 | 1521132 |
| AA126623 | 1687880 |
| AA062936 | 1557437 |
| AA062936 | 1557437 |
| AA147951 | 1717458 |
| AA148100 | 1717475 |
| AA281556 | 1924234 |
| AA482303 | 2209981 |
| AI276690 | 3898964 |
| AA303709 | 1956051 |
| AI004675 | 3214185 |
| AA039354 | 1515650 |
| AA563987 | 2335626 |
| AA488634 | 2216065 |
| AA399628 | 2052642 |
| AI267185 | 3886352 |
| AA226674 | 1847990 |
| AA469121 | 2195655 |
| AA652436 | 2584088 |
| AI671174 | 4850905 |
| AA446040 | 2158705 |
| AI309007 | 4003878 |
| AI201573 | 3754179 |
| C16203 | 1570910 |
| AA035003 | 1506966 |
| AA838431 | 2913230 |
| AA838431 | 2913230 |
| AA007569 | 1463565 |
| AA853131 | 2939870 |
| AA190559 | 1779534 |
| AA155915 | 1727533 |
| AI023372 | 3239778 |
| AI276690 | 3898964 |
| AI199515 | 3752121 |
| AI199515 | 3752121 |
| AI025517 | 3241130 |
| AA127565 | 1686938 |
| AA526498 | 2268567 |
| AA114138 | 1668024 |
| AA765228 | 2816466 |
| AA765228 | 2816466 |
| AI090077 | 3429136 |
| AA044748 | 1522951 |
| AA044748 | 1522951 |
| AI267502 | 3886669 |
| AA344583 | 1996819 |
| AA029043 | 1496652 |
| H52011 | 991852 |
| AA700350 | 2703313 |
| AA483053 | 2211898 |
| AA679015 | 2659537 |
| AA430369 | 2110944 |
| AI309007 | 4003878 |
| AA005232 | 1448694 |
| AA102563 | 1647755 |
| H98646 | 1123314 |
| AA548889 | 2319171 |
| AI267185 | 3886352 |
| W19131 | 1295138 |
| AA847560 | 2934078 |
| AA279747 | 1921212 |
| AA025721 | 1491497 |
| AA679015 | 2659537 |
| AA428184 | 2111834 |
| AI273856 | 3896124 |
| AA514959 | 2254559 |
| AA452335 | 2166004 |
| AA165463 | 1741479 |
| AA514542 | 2254142 |
| F32233 | 4817859 |
| AA400840 | 2054712 |
| AA385602 | 2038152 |
| AI075412 | 3399983 |
| AA772548 | 2824331 |
| AA772548 | 2824331 |
| AA679015 | 2659537 |
| AI267282 | 3886449 |
| AA075148 | 1615044 |
| AI624304 | 4649235 |
| AI436387 | 4281292 |
| AA166622 | 1745211 |
| AA916110 | 3055502 |
| AA047065 | 1524963 |
| AA399022 | 2052821 |
| AA136215 | 1697527 |
| AA039354 | 1515650 |
| AA634397 | 2557611 |
| AA788933 | 2849053 |
| AA039354 | 1515650 |
| AA323602 | 1975928 |
| AI332433 | 4068992 |
| AA446215 | 2158880 |
| AA082498 | 1624579 |
| AA464646 | 2189530 |
| AA465164 | 2191331 |
| AI754431 | 5132695 |
| AI754431 | 5132695 |
| H01401 | 864334 |
| H01401 | 864334 |
| AF001541 | 2529713 |
| AA047729 | 1527526 |
| AA399022 | 2052821 |
| AI127825 | 3596339 |
| AI089220 | 3428279 |
| AA770397 | 2821635 |
| AA305494 | 1958072 |
| AA083004 | 1625060 |
| AA151600 | 1720087 |
| AA233641 | 1856696 |
| AA226674 | 1847990 |
| AA453784 | 2167453 |
| AA853539 | 2940278 |
| AA449456 | 2162847 |
| AI625806 | 4650725 |
| AI348661 | 4085867 |
| AA608668 | 2457096 |
| AA303709 | 1956051 |
| AA612685 | 2463723 |
| AA113235 | 1664939 |
| AA576065 | 2350580 |
| A1033304 | 3254257 |
| AI090509 | 3429568 |
| D81636 | 1179513 |
| AA232447 | 1855234 |
| AA608790 | 2457218 |
| AA307513 | 1959842 |
| AI267185 | 3886352 |
| AI491897 | 4392900 |
| AA983883 | 3162408 |
| AA171834 | 1750944 |
| AI262064 | 3870267 |
| AI473830 | 4326875 |
| AA126418 | 1686030 |
| AA487845 | 2215276 |
| AA700903 | 2704068 |
| AA977584 | 3155030 |
| AA091539 | 1637002 |
| AA580412 | 2355739 |
| AA580412 | 2355739 |
| AA182766 | 1766475 |
| AA053874 | 1544801 |
| AI090863 | 3429922 |
| AI271883 | 3891050 |
| AA101862 | 1645220 |
| N24636 | 1138786 |
| AA112046 | 1664192 |
| AI421699 | 4267630 |
| AI738508 | 5100489 |
| AI738508 | 5100489 |
| AA464646 | 2189530 |
| AA234411 | 1858685 |
| AA595343 | 2410693 |
| AA314225 | 1966574 |
| AA706004 | 2715922 |
| AA704151 | 2714069 |
| AI267185 | 3886352 |
| AA075148 | 1615044 |
| AA026719 | 1492500 |
| AA420758 | 2094637 |
| AI362945 | 4114566 |
| AA936566 | 3094484 |
| AA063512 | 1557322 |
| AA112408 | 1664951 |
| AI478691 | 4371917 |
| AA694186 | 2695124 |
| AA026719 | 1492500 |
| AA541386 | 2287820 |
| T71404 | 685925 |
| T71404 | 685925 |
| AA228025 | 1849633 |
| AA228025 | 1849633 |
| AA493211 | 2223052 |
| AA228895 | 1851742 |
| AA398144 | 2051271 |
| AI309007 | 4003878 |
| AI127815 | 3596329 |
| AI127815 | 3596329 |
| AF001541 | 2529713 |
| AA047729 | 1527526 |
| AI262125 | 3870328 |
| AA459456 | 2184363 |
| AA459456 | 2184363 |
| AA913590 | 3052982 |
| AA913590 | 3052982 |
| AI241000 | 3836397 |
| AA769847 | 2821085 |
| AA522903 | 2263615 |
| AA725363 | 2743070 |
| AI093315 | 3432291 |
| AI188929 | 3740138 |
| H17020 | 883260 |
| AA573742 | 2348257 |
| AA307513 | 1959842 |
| AI144526 | 3666335 |
| AI167693 | 3700863 |
| AA506302 | 2242542 |
| W78198 | 1388230 |
| AA258725 | 1893849 |
| AI887267 | 5592431 |
| AA233109 | 1856103 |
| R22457 | 777238 |
| AI344311 | 4081517 |
| AI344311 | 4081517 |
| AA128261 | 1688329 |
| AI311789 | 4006660 |
| AA612685 | 2463723 |
| AA316516 | 1968844 |
| AA316516 | 1968844 |
| AA830249 | 2903348 |
| AA424829 | 2106952 |
| N46402 | 1187568 |
| AA946876 | 3110271 |
| AA411763 | 2070351 |
| AA057400 | 1550105 |
| AA609169 | 2457597 |
| AI310129 | 4005000 |
| AI679214 | 4889396 |
| AA831877 | 2904976 |
| AA653693 | 2589847 |
| N98658 | 1270080 |
| AA580144 | 2355471 |
| AA219116 | 1833343 |
| D81636 | 1179513 |
| R35844 | 792745 |
| AI493233 | 4394236 |
| AA714531 | 2726805 |
| AA531487 | 2274193 |
| AA765031 | 2816269 |
| AA765031 | 2816269 |
| AA191088 | 1779680 |
| AA193645 | 1783056 |
| AA376374 | 2028692 |
| AA864690 | 2959003 |
| AA453784 | 2167453 |
| AA453784 | 2167453 |
| W44970 | 1329071 |
| W44970 | 1329071 |
| H52011 | 991852 |
| AI678238 | 4888420 |
| AI360579 | 4112200 |
| H52011 | 991852 |
| H52011 | 991852 |
| AA286693 | 1932951 |
| AA233689 | 1856699 |
| AA453784 | 2167453 |
| AA453784 | 2167453 |
| AA788933 | 2849053 |
| AA349417 | 2001655 |
| AA101077 | 1647597 |
| AI141537 | 3648994 |
| AA075451 | 1615322 |
| AA258725 | 1893849 |
| AI309007 | 4003878 |
| AA526060 | 2268129 |
| AA256330 | 1891867 |
| AA156569 | 1728220 |
| AA130228 | 1691232 |
| D61455 | 970226 |
| AA864690 | 2959003 |
| AA349978 | 2002298 |
| AA446888 | 2159553 |
| AI254200 | 3861725 |
| AA151282 | 1719683 |
| AA614671 | 2466867 |
| AI494235 | 4395238 |
| AA410835 | 2069943 |
| AA090891 | 1635475 |
| AI392999 | 4222546 |
| AA604210 | 2445119 |
| AA974657 | 3149837 |
| AA765228 | 2816466 |
| AA070635 | 1577995 |
| AA974049 | 3149229 |
| AA644001 | 2569219 |
| AA037314 | 1512421 |
| AA037314 | 1512421 |
| AA214710 | 1814498 |
| AA196135 | 1791726 |
| AA227890 | 1849461 |
| AA451869 | 2165538 |
| AA769847 | 2821085 |
| T25387 | 2947324 |
| AI342714 | 4079920 |
| AI342714 | 4079920 |
| AA315103 | 1967442 |
| AA453217 | 2166886 |
| AA349978 | 2002298 |
| AI652058 | 4736037 |
| AI052317 | 3308308 |
| AA082498 | 1624579 |
| AA662648 | 2616639 |
| AA225164 | 1846568 |
| AA092923 | 1638000 |
| AI193577 | 3744786 |
| N78081 | 1240782 |
| AA243394 | 1874187 |
| AA100384 | 1646757 |
| AI609252 | 4618419 |
| AI609252 | 4618419 |
| AA075507 | 1615377 |
| AA961734 | 3133898 |
| AA148011 | 1717402 |
| AI077325 | 3411733 |
| AI077325 | 3411733 |
| AA523877 | 2264805 |
| AA039354 | 1515650 |
| AA101077 | 1647597 |
| AA083471 | 1625557 |
| AA723444 | 2741151 |
| AA788933 | 2849053 |
| AA552818 | 2323072 |
| AA088200 | 1633703 |
| AA021648 | 1485309 |
| AA333713 | 1985957 |
| AA702428 | 2705541 |
| AI342714 | 4079920 |
| Z44537 | 573675 |
| AA424680 | 2103624 |
| AA252440 | 1887411 |
| AI004348 | 3213858 |
| AA738116 | 2768873 |
| AA454514 | 2177290 |
| AA082795 | 1624852 |
| AA242997 | 1873996 |
| AA216022 | 1815993 |
| AA157054 | 1728713 |
| AI278872 | 3917106 |
| AA258725 | 1893849 |
| AA258725 | 1893849 |
| AA573742 | 2348257 |
| AA165638 | 1741671 |
| AI435429 | 4303120 |
| AA639701 | 2563480 |
| AA788933 | 2849053 |
| AA707546 | 2717464 |
| AA243767 | 1874569 |
| AA243767 | 1874569 |
| AA300170 | 1952574 |
| AA678586 | 2659108 |
| AI435276 | 4302048 |
| AA157680 | 1729295 |
| AA131338 | 1692836 |
| AA704512 | 2714430 |
| AI630387 | 4681717 |
| AA255567 | 1892326 |
| AA255567 | 1892326 |
| N46402 | 1187568 |
| AA531504 | 2274210 |
| AI351167 | 4088373 |
| AA523877 | 2264805 |
| AA181734 | 1765218 |
| AA608623 | 2457051 |
| D81635 | 1179512 |
| AI148825 | 3677294 |
| AA122401 | 1678777 |
| AA186758 | 1774858 |
| AA074086 | 1613956 |
| AA216132 | 1816071 |
| AA131231 | 1692739 |
| AA769937 | 2821175 |
| AA376374 | 2028692 |
| AA412184 | 2070755 |
| AA788933 | 2849053 |
| AA258116 | 1894250 |
| R54092 | 815994 |
| AA306488 | 1959058 |
| H52011 | 991852 |
| AA947616 | 3108869 |
| AA317903 | 1970229 |
| AA079835 | 1618787 |
| AI131245 | 3601261 |
| AA136215 | 1697527 |
| AA113359 | 1665199 |
| AA375933 | 2028282 |
| AA469304 | 2195838 |
| AA521069 | 2261612 |
| AA111853 | 1663940 |
| D14533 | 286028 |
| X13111 | 32138 |
| K00799 | 182681 |
| D10522 | 219893 |
| AB007952 | 3413925 |
| X04408 | 31914 |
| X04408 | 31914 |
| AB002368 | 2224680 |
| X73608 | 793844 |
| X73608 | 793844 |
| M55409 | 189596 |
| D83327 | 2687860 |
| M10905 | 182696 |
| M30257 | 179885 |
| AF086204 | 3483549 |
| AF007216 | 2281471 |
| M77830 | 4689438 |
| AF052113 | 3360420 |
| D38583 | 560790 |
| L13385 | 349823 |
| L13385 | 349823 |
| M17885 | 190231 |
| AF016535 | 2353263 |
| AF016535 | 2353263 |
| AF068302 | 4584876 |
| X04098 | 28338 |
| L10612 | 402701 |
| X07549 | 29707 |
| M31470 | 190880 |
| M95724 | 180246 |
| AJ010841 | 3646127 |
| Y07968 | 1552327 |
| AF035286 | 2661038 |
| AB024704 | 4589928 |
| D00726 | 219655 |
| J02943 | 179970 |
| J02943 | 179970 |
| X87949 | 1143491 |
| M10941 | 180422 |
| U51586 | 1809247 |
| AF004429 | 2895082 |
| E02628 | 2170856 |
| D87432 | 1665758 |
| X07897 | 37207 |
| U39318 | 1145690 |
| J03202 | 186916 |
| J03202 | 186916 |
| U34074 | 1049216 |
| U42404 | 4096845 |
| AF121860 | 4689259 |
| D50918 | 1469178 |
| D50918 | 1469178 |
| X04098 | 28338 |
| X04098 | 28338 |
| U14968 | 550016 |
| U42404 | 4096845 |
| AB014577 | 3327167 |
| U42404 | 4096845 |
| U42404 | 4096845 |
| X64707 | 29382 |
| AF127918 | 4761562 |
| AF127918 | 4761562 |
| E01650 | 2169903 |
| M64241 | 190813 |
| M64241 | 190813 |
| E02628 | 2170856 |
| X04098 | 28338 |
| AL050161 | 4884375 |
| U42404 | 4096845 |
| X03963 | 30091 |
| D86322 | 2467376 |
| U42404 | 4096845 |
| U42404 | 4096845 |
| U51586 | 1809247 |
| M34788 | 182495 |
| M34788 | 182495 |
| M58297 | 189043 |
| AF035286 | 2661038 |
| M10905 | 182696 |
| X04098 | 28338 |
| U05598 | 531159 |
| AF083322 | 3435243 |
| X04098 | 28338 |
| X77956 | 457784 |
| AJ000147 | 2370089 |
| M58297 | 189043 |
| E02628 | 2170856 |
| AB002533 | 11275292 |
| U42404 | 4096845 |
| X03963 | 30091 |
| L11066 | 307322 |
| D17268 | 598899 |
| AL050179 | 4884392 |
| AB007896 | 2662152 |
| U07919 | 995897 |
| K00799 | 182681 |
| U61837 | 2668504 |
| AF083322 | 3435243 |
| U42404 | 4096845 |
| U42404 | 4096845 |
| X85373 | 806565 |
| E02326 | 2170561 |
| Z11695 | 23878 |
| U07919 | 995897 |
| AB014511 | 3327035 |
| AB014511 | 3327035 |
| X79535 | 496886 |
| X79535 | 496886 |
| U42404 | 4096845 |
| U42404 | 4096845 |
| M11353 | 184092 |
| AF070561 | 3387928 |
| AF070561 | 3387928 |
| AL021683 | 3217033 |
| M27508 | 179420 |
| M22590 | 179418 |
| AF070648 | 3283922 |
| AJ010841 | 3646127 |
| M23254 | 511636 |
| D80005 | 1136425 |
| M94345 | 187455 |
| AF070524 | 3387879 |
| L42542 | 974142 |
| U42404 | 4096845 |
| U42404 | 4096845 |
| AF070524 | 3387879 |
| AF070524 | 3387879 |
| AF028832 | 3287488 |
| U42457 | 4098551 |
| K00799 | 182681 |
| M96982 | 338262 |
| X85373 | 806565 |
| L33930 | 500848 |
| M22590 | 179418 |
| M22590 | 179418 |
| X84407 | 666046 |
| U42404 | 4096845 |
| X79535 | 496886 |
| AB002533 | 11275292 |
| X84407 | 666046 |
| X13238 | 1200056 |
| AF086336 | 3483681 |
| AB021654 | 4062862 |
| U07919 | 995897 |
| AJ223812 | 2894518 |
| U42404 | 4096845 |
| X07897 | 37207 |
| U42404 | 4096845 |
| AB019564 | 3885367 |
| M27508 | 179420 |
| AF070561 | 3387928 |
| AF070561 | 3387928 |
| D87930 | 2443337 |
| E01650 | 2169903 |
| U42457 | 4096851 |
| D25542 | 662389 |
| AB004903 | 2443360 |
| AB004903 | 2443360 |
| M37583 | 184059 |
| M69181 | 641957 |
| M69181 | 641957 |
| M69181 | 641957 |
| S80562 | 1245966 |
| U84573 | 2138313 |
| AF028832 | 3287488 |
| M33308 | 340236 |
| AL035081 | 4107432 |
| AF070664 | 4454703 |
| AJ011007 | 4468340 |
| AB018346 | 3882326 |
| D42054 | 577310 |
| M58510 | 896472 |
| K00799 | 182681 |
| E02326 | 2170561 |
| M16279 | 188542 |
| U42404 | 4096845 |
| AL035081 | 4107432 |
| AB007862 | 2662084 |
| E01650 | 2169903 |
| E01650 | 2169903 |
| M23254 | 511636 |
| M23254 | 511636 |
| AB003102 | 1945608 |
| L25610 | 425142 |
| M16279 | 188542 |
| J02943 | 179970 |
| AL049974 | 4884224 |
| AF098462 | 4050037 |
| M15502 | 182793 |
| E01650 | 2169903 |
| AF084260 | 3514096 |
| AF120268 | 4835848 |
| AJ010841 | 3646127 |
| AJ010841 | 3646127 |
| U42404 | 4096845 |
| U90545 | 2062691 |
| X07897 | 37207 |
| AB007862 | 2662084 |
| AF011468 | 3213196 |
| L38933 | 1008841 |
| U07343 | 463988 |
| M69066 | 188625 |
| L05093 | 401844 |
| X04098 | 28338 |
| AF143324 | 4929225 |
| D12676 | 219702 |
| Y11312 | 2808446 |
| U14970 | 550020 |
| X81198 | 773574 |
| AF021819 | 2460317 |
| J03576 | 189753 |
| M10905 | 182696 |
| U42404 | 4096845 |
| U42404 | 4096845 |
| U42404 | 4096845 |
| AB018346 | 3882326 |
| AB012083 | 3551145 |
| X02761 | 31396 |
| X81198 | 773574 |
| U64898 | 2897866 |
| D64015 | 2281005 |
| Y09565 | 2894089 |
| AF007145 | 2852622 |
| E01650 | 2169903 |
| U57847 | 1373420 |
| M69066 | 188625 |
| U41850 | 4096826 |
| AJ006834 | 3236104 |
| D12485 | 219943 |
| AJ010841 | 3646127 |
| AB019568 | 3885371 |
| AB014560 | 3327133 |
| X97064 | 1296663 |
| U42404 | 4096845 |
| J00127 | 182423 |
| AF086002 | 3483347 |
| U14969 | 550018 |
| U42404 | 4096845 |
| AF119386 | 4877697 |
| AB002533 | 11275292 |
| J03634 | 181946 |
| M74775 | 187151 |
| D26124 | 556515 |
| J03460 | 189963 |
| AF151885 | 4929722 |
| D49737 | 2588778 |
| U42404 | 4096845 |
| AF116827 | 4768830 |
| M14083 | 189566 |
| Z47087 | 860989 |
| M23254 | 511636 |
| AF054990 | 3005703 |
| M22324 | 178535 |
| AF077045 | 4689137 |
| U42457 | 4096851 |
| AB020660 | 4240194 |
| D80005 | 1136425 |
| X64707 | 29382 |
| AL021683 | 3217033 |
| AF151885 | 4929722 |
| D32000 | 507327 |
| S82470 | 1699264 |
| AL049381 | 4500168 |
| X81198 | 773574 |
| D50918 | 1469178 |
| D50918 | 1469178 |
| AF070561 | 3387928 |
| S78569 | 1042081 |
| U02390 | 409928 |
| U34074 | 1049216 |
| U33760 | 995823 |
| E03413 | 2171629 |
| J03202 | 186916 |
| M55268 | 177837 |
| J03537 | 337513 |
| D21260 | 434760 |
| AF044671 | 4105274 |
| AF044671 | 4105274 |
| M22918 | 189019 |
| AF054990 | 3005703 |
| M69181 | 641957 |
| AF006084 | 2282033 |
| L16785 | 349475 |
| Y00052 | 30308 |
| U57847 | 1373420 |
| M69181 | 641957 |
| X69970 | 32461 |
| X69970 | 32461 |
| AB018346 | 3882326 |
| AF077045 | 4689137 |
| AF077043 | 4689133 |
| AF054179 | 3341999 |
| AF054179 | 3341999 |
| D63998 | 974733 |
| D63998 | 974733 |
| AB000220 | 3426162 |
| E03157 | 2171374 |
| M33308 | 340236 |
| L34600 | 609491 |
| AF039022 | 2873376 |
| X07897 | 37207 |
| AB014548 | 3327109 |
| AL021683 | 3217033 |
| D86957 | 1503987 |
| J04543 | 338243 |
| L13806 | 306554 |
| U12465 | 562073 |
| X87176 | 1050516 |
| U42458 | 4096853 |
| AB007877 | 2887408 |
| AB002533 | 11275292 |
| AB002533 | 11275292 |
| X64707 | 29382 |
| M21551 | 189227 |
| D49737 | 2588778 |
| AJ223500 | 2815295 |
| D13641 | 285986 |
| L34600 | 609491 |
| AF039022 | 2873376 |
| M69181 | 641957 |
| AF147319 | 4761670 |
| M10119 | 182517 |
| M10119 | 182517 |
| U41515 | 1209723 |
| J03202 | 186916 |
| X83218 | 1008079 |
| J00127 | 182423 |
| J04543 | 338243 |
| M69181 | 641957 |
| K00799 | 182681 |
| D50918 | 1469178 |
| D83198 | 3893156 |
| D83198 | 3893156 |
| AB011115 | 3043609 |
| AB011115 | 3043609 |
| AF070638 | 3283909 |
| AF070638 | 3283909 |
| L28010 | 452047 |
| L28010 | 452047 |
| AF031385 | 2606093 |
| AF031385 | 2606093 |
| AB002386 | 2224716 |
| AB002386 | 2224716 |
| X98248 | 9716091 |
| U02032 | 404014 |
| K03195 | 183302 |
| U42404 | 4096845 |
| U42404 | 4096845 |
| S56985 | 298485 |
| AF077611 | 3348070 |
| 049950 | 1405318 |
| J03460 | 189963 |
| AF070649 | 3283923 |
| M24194 | 187701 |
| X13238 | 1200056 |
| X13238 | 1200056 |
| M11058 | 184243 |
| D37991 | 1019367 |
| AL050161 | 4884375 |
| AJ002030 | 2570006 |
| AB002533 | 11275292 |
| Y00503 | 34038 |
| D87665 | 1620014 |
| D87665 | 1620014 |
| D29992 | 484050 |
| K00799 | 182681 |
| Y00345 | 35569 |
| D50918 | 1469178 |
| D50918 | 1469178 |
| L05095 | 388034 |
| AF077045 | 4689137 |
| M22920 | 189021 |
| AL050218 | 4884459 |
| M81757 | 337732 |
| L13210 | 307152 |
| X03963 | 30091 |
| AL050018 | 4884085 |
| Y00819 | 37508 |
| M17733 | 339688 |
| U94586 | 1946691 |
| U94586 | 1946691 |
| J00129 | 182429 |
| U42404 | 4096845 |
| U42404 | 4096845 |
| X51473 | 31410 |
| 029643 | 473936 |
| K00799 | 182681 |
| X79535 | 496886 |
| X79535 | 496886 |
| AF020797 | 9256827 |
| AB002533 | 11275292 |
| AB002533 | 11275292 |
| M69181 | 641957 |
| M69181 | 641957 |
| M94345 | 187455 |
| AF029890 | 2745882 |
| AF029890 | 2745882 |
| X56998 | 37564 |
| X56998 | 37564 |
| D87665 | 1620014 |
| D87665 | 1620014 |
| J00127 | 182423 |
| M58569 | 182406 |
| M16247 | 178044 |
| M16247 | 178044 |
| X69392 | 36114 |
| U73778 | 1846004 |
| U42457 | 4096851 |
| D16892 | 598649 |
| X15187 | 37260 |
| M14083 | 189566 |
| Z82022 | 2239118 |
| D89667 | 1731808 |
| J03460 | 189963 |
| J02611 | 178840 |
| AF145316 | 4929324 |
| U42404 | 4096845 |
| U42404 | 4096845 |
| AF070561 | 3387928 |
| AF070561 | 3387928 |
| AF070649 | 3283923 |
| AB019568 | 3885371 |
| J03634 | 181946 |
| J03634 | 181946 |
| AJ010841 | 3646127 |
| D37766 | 2429078 |
| AF054990 | 3005703 |
| X79535 | 496886 |
| U05598 | 531159 |
| AF007791 | 3779196 |
| AF038451 | 3779225 |
| J03592 | 339722 |
| X87176 | 1050516 |
| U42404 | 4096845 |
| U42594 | 4096861 |
| D29992 | 484050 |
| AL050218 | 4884459 |
| AL021683 | 3217033 |
| M10119 | 182517 |
| M10119 | 182517 |
| D87454 | 1665796 |
| D87454 | 1665796 |
| AB019568 | 3885371 |
| M22920 | 189021 |
| X07979 | 31441 |
| AL050209 | 4884448 |
| AL050209 | 4884448 |
| AB014577 | 3327167 |
| AB014577 | 3327167 |
| AJ010841 | 3646127 |
| AF045606 | 8277249 |
| AF086280 | 3483625 |
| AB014548 | 3327109 |
| AF132959 | 4680688 |
| D50918 | 1469178 |
| X87176 | 1050516 |
| M24630 | 514363 |
| E01650 | 2169903 |
| M69181 | 641957 |
| AJ010841 | 3646127 |
| AB012664 | 3702223 |
| J03202 | 186916 |
| AF017789 | 2460123 |
| AF017789 | 2460123 |
| X56999 | 37568 |
| AF035319 | 2661082 |
| K03515 | 189237 |
| U39318 | 1145690 |
| X87176 | 1050516 |
| X87176 | 1050516 |
| AB007896 | 2662152 |
| AF001434 | 2529706 |
| AB007896 | 2662152 |
| AB007896 | 2662152 |
| D50525 | 1167502 |
| Z24725 | 505032 |
| AB000220 | 3426162 |
| AB000220 | 3426162 |
| D16234 | 303617 |
| D16234 | 303617 |
| M55265 | 177993 |
| J03779 | 179833 |
| U90545 | 2062691 |
| AF026166 | 4090928 |
| E03157 | 2171374 |
| X60489 | 31099 |
| AB007883 | 2887420 |
| AB007883 | 2887420 |
| M10905 | 182696 |
| AF026166 | 4090928 |
| X63692 | 1632818 |
| K00799 | 182681 |
| X15187 | 37260 |
| X15187 | 37260 |
| X63692 | 1632818 |
| K00799 | 182681 |
| M10905 | 182696 |
| AB011128 | 3043635 |
| X02761 | 31396 |
| L27211 | 558656 |
| AF077043 | 4689133 |
| EQ1797 | 2170049 |
| J04031 | 187464 |
| AB011128 | 3043635 |
| X76302 | 431952 |
| D89053 | 4165017 |
| M24194 | 187701 |
| U64898 | 2897866 |
| X93207 | 2462486 |
| M10905 | 182696 |
| J03202 | 186916 |
| D89053 | 4165017 |
| D13642 | 285998 |
| J03537 | 337513 |
| D64015 | 2281005 |
| D64015 | 2281005 |
| AF000421 | 2183080 |
| AF000421 | 2183080 |
| M22920 | 189021 |
| M22918 | 189019 |
| D29992 | 484050 |
| D29992 | 484050 |
| J03202 | 186916 |
| J03202 | 186916 |
| M17885 | 190231 |
| AF046025 | 4581063 |
| AF046025 | 4581063 |
| J02943 | 179970 |
| J02943 | 179970 |
| AL021683 | 3217033 |
| J03040 | 338312 |
| AJ010841 | 3646127 |
| AJ010841 | 3646127 |
| U05598 | 531159 |
| AB021654 | 4062862 |
| J02943 | 179970 |
| D38255 | 2463543 |
| D84212 | 2641947 |
| AF008551 | 2979627 |
| L33930 | 500848 |
| J03202 | 186916 |
| J03202 | 186916 |
| Z22555 | 397606 |
| K00799 | 182681 |
| X02761 | 31396 |
| X63432 | 28335 |
| X51473 | 31410 |
| X81198 | 773574 |
| D13388 | 219587 |
| AB018346 | 3882326 |
| X70326 | 38434 |
| M23254 | 511636 |
| AF086003 | 3483348 |
| K00799 | 182681 |
| X67698 | 37476 |
| X67698 | 37476 |
| M10941 | 180422 |
| E01650 | 2169903 |
| AF086336 | 3483681 |
| AJ011007 | 4468340 |
| U76421 | 2039299 |
| D29992 | 484050 |
| AB001106 | 1850083 |
| J00129 | 182429 |
| AF052164 | 3360475 |
| U61836 | 1470083 |
| K02765 | 179664 |
| AB006679 | 2342476 |
| M10905 | 182696 |
| AF077045 | 4689137 |
| AF089747 | 4165889 |
| J03460 | 189963 |
| J03460 | 189963 |
| AB007862 | 2662084 |
| AB007862 | 2662084 |
| AB018346 | 3882326 |
| AB019568 | 3885371 |
| AF000982 | 2580549 |
| J00127 | 182423 |
| D45198 | 971271 |
| M15990 | 181267 |
| M15990 | 181267 |
| AB010812 | 4521224 |
| U63139 | 1518805 |
| AF028832 | 3287488 |
| AF145316 | 4929324 |
| E01816 | 2170068 |
| Y00281 | 36052 |
| AF035752 | 2665791 |
| U94586 | 1946691 |
| AB019568 | 3885371 |
| J04823 | 1311703 |
| L43964 | 951202 |
| L43964 | 951202 |
| M31523 | 339477 |
| AF007128 | 2852601 |
| AF023611 | 2565274 |
| AF031385 | 2606093 |
| L37368 | 1236282 |
| J03460 | 189963 |
| L13286 | 306703 |
| AL050380 | 4914582 |
| AL050380 | 4914582 |
| U33760 | 995823 |
| AB019564 | 3885367 |
| M27504 | 339809 |
| M27504 | 339809 |
| AF030555 | 3158350 |
| AF030555 | 3158350 |
| M37583 | 184059 |
| AF047002 | 2896145 |
| AF086513 | 3483858 |
| AJ002030 | 2570008 |
| U85755 | 4099300 |
| L05425 | 179284 |
| L05425 | 179284 |
| X07897 | 37207 |
| X07897 | 37207 |
| K03515 | 189237 |
| K03515 | 189237 |
| X70394 | 468707 |
| X70394 | 468707 |
| M10905 | 182696 |
| U41850 | 4096826 |
| L22154 | 347385 |
| AJ004913 | 3288462 |
| AF064093 | 3323608 |
| S80343 | 1217667 |
| D13641 | 285986 |
| D13641 | 285986 |
| AF016535 | 2353263 |
| S68330 | 544764 |
| L05425 | 179284 |
| AF054987 | 3005697 |
| AF054987 | 3005697 |
| AL049386 | 4500176 |
| AB019564 | 3885367 |
| AF054990 | 3005703 |
| AB007931 | 3413885 |
| M74524 | 184043 |
| U76764 | 1685050 |
| X05908 | 34387 |
| J02943 | 179970 |
| AF117815 | 4235629 |
| J03537 | 337513 |
| AF070550 | 3387913 |
| J00127 | 182423 |
| U42594 | 4096861 |
| J03460 | 189963 |
| X53331 | 34613 |
| M58549 | 187592 |
| M81757 | 337732 |
| M86667 | 189066 |
| L06328 | 340200 |
| L08666 | 190199 |
| AJ011007 | 4468340 |
| X07897 | 37207 |
| AF035752 | 2665791 |
| AF077205 | 4679023 |
| D13748 | 219402 |
| D13748 | 219402 |
| X63527 | 36127 |
| D14665 | 6630617 |
| D14665 | 6630617 |
| X64707 | 29382 |
| AF132000 | 4530586 |
| AF132000 | 4530586 |
| X87949 | 1143491 |
| M31899 | 182178 |
| M31899 | 182178 |
| X63432 | 28335 |
| V00478 | 28244 |
| U42594 | 4096861 |
| U42594 | 4096861 |
| AF039656 | 2773159 |
| U84573 | 2138313 |
| U84573 | 2138313 |
| D55654 | 1255603 |
| AL050161 | 4884375 |
| AL050380 | 4914582 |
| U34360 | 1144492 |
| AJ010841 | 3646127 |
| AB020684 | 4240242 |
| AB020684 | 4240242 |
| U63139 | 1518805 |
| U63139 | 1518805 |
| M80359 | 189511 |
| M80359 | 189511 |
| X97065 | 1296665 |
| X97065 | 1296665 |
| U94586 | 1946691 |
| U05598 | 531159 |
| AF070672 | 3978239 |
| AF070672 | 3978239 |
| AF098865 | 4204674 |
| AF098865 | 4204674 |
| AF070561 | 3387928 |
| AF070550 | 3387913 |
| AF070550 | 3387913 |
| J03537 | 337513 |
| AF069072 | 3929113 |
| AF069072 | 3929113 |
| AF038451 | 3779225 |
| AF007791 | 3779196 |
| U77085 | 1684789 |
| AF070561 | 3387928 |
| AF070561 | 3387928 |
| X79535 | 496886 |
| M10905 | 182696 |
| AF039656 | 2773159 |
| U39360 | 1066079 |
| U39360 | 1066079 |
| D78335 | 1655419 |
| U76764 | 1685050 |
| AB011108 | 3043595 |
| AL049974 | 4884224 |
| J03202 | 186916 |
| J03202 | 186916 |
| AF077043 | 4689133 |
| AF077043 | 4689133 |
| U84573 | 2138313 |
| U02032 | 404014 |
| Z28407 | 433898 |
| K00799 | 182681 |
| U25766 | 808064 |
| U25766 | 808064 |
| AB017363 | 3927882 |
| J03537 | 337513 |
| L31610 | 1220360 |
| S60099 | 300168 |
| AB007862 | 2662084 |
| AB007862 | 2662084 |
| AF031385 | 2606093 |
| J02814 | 189875 |
| J02814 | 189875 |
| U79291 | 1710271 |
| AF077045 | 4689137 |
| AF077045 | 4689137 |
| M94345 | 187455 |
| AF064093 | 3323608 |
| AF064093 | 3323608 |
| X04098 | 28338 |
| J00129 | 182429 |
| M58569 | 182406 |
| AB020660 | 4240194 |
| M94345 | 187455 |
| J04991 | 189425 |
| AB014577 | 3327167 |
| AL049339 | 4500122 |
| AB014512 | 3327037 |
| X13709 | 35389 |
| M19961 | 180940 |
| M22920 | 189021 |
| K00799 | 182681 |
| X02761 | 31396 |
| S75169 | 896223 |
| AF085361 | 5114056 |
| J00127 | 182423 |
| M24486 | 190785 |
| J03460 | 189963 |
| M10119 | 182517 |
| E02628 | 2170856 |
| E01650 | 2169903 |
| J03202 | 186916 |
| J03202 | 186916 |
| U41515 | 1209723 |
| M24630 | 514363 |
| M24630 | 514363 |
| S59184 | 299788 |
| X69970 | 32461 |
| D50312 | 1109633 |
| D50312 | 1109633 |
| AB000220 | 3426162 |
| AB000220 | 3426162 |
| D87453 | 1665794 |
| U14391 | 557467 |
| U14391 | 557467 |
| AF073298 | 3641537 |
| AF086336 | 3483681 |
| AF086336 | 3483681 |
| AF000982 | 2580549 |
| AF000982 | 2580549 |
| U19252 | 808035 |
| U19252 | 808035 |
| J03799 | 186840 |
| D50371 | 2605591 |
| U12404 | 531170 |
| AF086557 | 3483902 |
| X56932 | 23690 |
| J02943 | 179970 |
| D50371 | 2605591 |
| AB028624 | 5103045 |
| M65217 | 184404 |
| U30246 | 903681 |
| U30246 | 903681 |
| X02761 | 31398 |
| AB002533 | 11275292 |
| AB002533 | 11275292 |
| D49737 | 2588778 |
| X07897 | 37207 |
| M55268 | 177837 |
| S66431 | 435777 |
| S66431 | 435777 |
| U00947 | 405049 |
| M33308 | 340236 |
| M33308 | 340236 |
| J04543 | 338243 |
| M33308 | 340236 |
| M10905 | 182696 |
| L16785 | 349475 |
| L16785 | 349475 |
| U63139 | 1518805 |
| U63139 | 1518805 |
| AF088071 | 3523277 |
| U15008 | 600747 |
| X04098 | 28338 |
| X04098 | 28338 |
| D63476 | 1469865 |
| X79535 | 496886 |
| D50918 | 1469178 |
| D50918 | 1469178 |
| U42404 | 4096845 |
| D13641 | 285986 |
| D13641 | 285986 |
| AB023151 | 4589511 |
| AB023151 | 4589511 |
| U42594 | 4096861 |
| U42594 | 4096861 |
| U42457 | 4096851 |
| E01816 | 2170068 |
| U42404 | 4096845 |
| U42404 | 4096845 |
| AF086336 | 3483681 |
| AF086336 | 3483681 |
| U42404 | 4096845 |
| J04621 | 184428 |
| AF039656 | 2773159 |
| D50918 | 1469178 |
| AF001601 | 2228774 |
| AF001601 | 2228774 |
| AB014577 | 3327167 |
| AB014577 | 3327167 |
| AF054183 | 4092053 |
| AF052578 | 2967847 |
| M12623 | 184233 |
| AF151832 | 4929616 |
| AF007170 | 2865251 |
| AF007170 | 2865251 |
| AF054987 | 3005697 |
| AF054987 | 3005697 |
| J02943 | 179970 |
| L20941 | 507251 |
| L20941 | 507251 |
| M27504 | 339809 |
| M27504 | 339809 |
| AF104419 | 4106877 |
| M80359 | 189511 |
| M80359 | 189511 |
| AF039656 | 2773159 |
| U57847 | 1373420 |
| U57847 | 1373420 |
| D13642 | 285998 |
| X02761 | 31396 |
| Y08982 | 3256190 |
| AJ010841 | 3646127 |
| AJ002030 | 2570006 |
| AJ002030 | 2570006 |
| AB019568 | 3885371 |
| AB021654 | 4062862 |
| U05598 | 531159 |
| AF099149 | 3930775 |
| L37368 | 1236282 |
| L37368 | 1236282 |
| AB019564 | 3885367 |
| J00128 | 182425 |
| J00127 | 182423 |
| D90209 | 220087 |
| D90209 | 220087 |
| AB007862 | 2662084 |
| AB007862 | 2662084 |
| E01954 | 2170202 |
| E01954 | 2170202 |
| AB020692 | 4240258 |
| AB020692 | 4240258 |
| J04973 | 180927 |
| D63486 | 1469885 |
| D45906 | 1805593 |
| J00127 | 182423 |
| J00127 | 182423 |
| U12465 | 562073 |
| U12465 | 562073 |
| K03515 | 189237 |
| AB020662 | 4240198 |
| AB002322 | 6634014 |
| K03515 | 189237 |
| K03515 | 189237 |
| U30246 | 903681 |
| U30246 | 903681 |
| AF147331 | 4761682 |
| X00570 | 28802 |
| AF077599 | 3342561 |
| J00129 | 182429 |
| J00129 | 182429 |
| U42404 | 4096845 |
| U42404 | 4096845 |
| AJ002030 | 2570006 |
| AJ002030 | 2570006 |
| AB023195 | 4589599 |
| AB023195 | 4589599 |
| D50310 | 1183161 |
| U39402 | 4096747 |
| X87949 | 1143491 |
| M10119 | 182517 |
| M10119 | 182517 |
| AF070550 | 3387913 |
| AF070550 | 3387913 |
| AF131781 | 4406608 |
| AF131781 | 4406608 |
| U53476 | 2105099 |
| M10905 | 182696 |
| X02761 | 31396 |
| Y00503 | 34038 |
| J03040 | 338312 |
| U42404 | 4096845 |
| U42404 | 4096845 |
| AF092128 | 5138905 |
| J03796 | 182072 |
| J03796 | 182072 |
| AF070606 | 3387987 |
| M17885 | 190231 |
| D63486 | 1469885 |
| D63486 | 1469885 |
| D87127 | 1817551 |
| D87127 | 1817551 |
| J02943 | 179970 |
| J02943 | 179970 |
| AB001106 | 1850083 |
| M10905 | 182696 |
| D45906 | 1805593 |
| AF092133 | 5138915 |
| J03537 | 337513 |
| M58485 | 180154 |
| AF077205 | 4679023 |
| AF077205 | 4679023 |
| AF007791 | 3779196 |
| AF007791 | 3779196 |
| D49737 | 2588778 |
| D49737 | 2588778 |
| X15880 | 30029 |
| X77588 | 517484 |
| U03272 | 437971 |
| AF086336 | 3483681 |
| D49489 | 1136742 |
| S78085 | 998900 |
| X00570 | 28802 |
| J03460 | 189963 |
| AF007791 | 3779196 |
| U77085 | 1684789 |
| U77085 | 1684789 |
| X07979 | 31441 |
| D83776 | 1228034 |
| D83776 | 1228034 |
| AF086336 | 3483681 |
| AF086336 | 3483681 |
| AF026004 | 2570863 |
| AF077044 | 4689135 |
| AF077044 | 4689135 |
| AF151856 | 4929664 |
| AF151856 | 4929664 |
| AB018346 | 3882326 |
| AB018346 | 3882326 |
| U78575 | 1743870 |
| U78578 | 1743876 |
| M10905 | 182696 |
| K00799 | 182681 |
| K00799 | 182681 |
| L05425 | 179284 |
| L05425 | 179284 |
| AF086336 | 3483681 |
| AJ011007 | 4468340 |
| L19184 | 440305 |
| X67951 | 287640 |
| AF038451 | 3779225 |
| M29960 | 339886 |
| AF086336 | 3483681 |
| AF086336 | 3483681 |
| Y00345 | 35569 |
| Y00345 | 35569 |
| J03202 | 186916 |
| AF086183 | 3483528 |
| AF086183 | 3483528 |
| X77588 | 517484 |
| U14528 | 549987 |
| X17206 | 34391 |
| M81757 | 337732 |
| M81757 | 337732 |
| J03202 | 186916 |
| J03202 | 186916 |
| AF007791 | 3779196 |
| AF038451 | 3779225 |
| AF007791 | 3779196 |
| M98479 | 179410 |
| E01650 | 2169903 |
| AF114264 | 4768674 |
| U42404 | 4096845 |
| U42404 | 4096845 |
| AB002533 | 11275292 |
| Y08982 | 3256190 |
| AF086172 | 3483517 |
| X02761 | 31396 |
| U30521 | 963091 |
| U30521 | 963091 |
| X63527 | 36127 |
| L05092 | 388031 |
| L05092 | 388031 |
| Z24725 | 505032 |
| X54942 | 29978 |
| X54942 | 29978 |
| AB002439 | 2943814 |
| X81889 | 1702923 |
| X81889 | 1702923 |
| AF007150 | 2852628 |
| L10910 | 405191 |
| AL049339 | 4500122 |
| X55740 | 23896 |
| X55740 | 23896 |
| AF143096 | 4689435 |
| AF143096 | 4689435 |
| AB020684 | 4240242 |
| U42457 | 4096851 |
| Z31696 | 479156 |
| AF098865 | 4204674 |
| AF098865 | 4204674 |
| U12404 | 531170 |
| S75169 | 896223 |
| AF070672 | 3978239 |
| AF153329 | 5007077 |
| U05040 | 460151 |
| AF086336 | 3483681 |
| AF132959 | 4680688 |
| U47077 | 9027566 |
| U34994 | 6525311 |
| J03460 | 189963 |
| U06452 | 476131 |
| U06452 | 476131 |
| M34788 | 182495 |
| M34788 | 182495 |
| D21260 | 434760 |
| D21260 | 434760 |
| AF039575 | 2773157 |
| D55671 | 870742 |
| X16312 | 29964 |
| J03202 | 186916 |
| J03202 | 186916 |
| AF077045 | 4689137 |
| D29992 | 484050 |
| AF070606 | 3387987 |
| AF070606 | 3387987 |
| K00799 | 182681 |
| K00799 | 182681 |
| X07979 | 31441 |
| E02628 | 2170856 |
| AF016582 | 2367668 |
| AF016582 | 2367668 |
| U47101 | 1685101 |
| U47101 | 1685101 |
| L05425 | 179284 |
| AJ010841 | 3646127 |
| U70660 | 1945364 |
| AF052178 | 3360489 |
| M98479 | 179410 |
| AF151856 | 4929664 |
| AF151856 | 4929664 |
| AF14Th31 | 4761682 |
| K00799 | 182681 |
| K00799 | 182681 |
| D63486 | 1469885 |
| D63486 | 1469885 |
| D50371 | 2605591 |
| AB028624 | 5103045 |
| AF020797 | 9256827 |
| AB014577 | 3327167 |
| AB014577 | 3327167 |
| AB014512 | 3327037 |
| M69181 | 641957 |
| M69181 | 641957 |
| U42404 | 4096845 |
| U42404 | 4096845 |
| D13641 | 285986 |
| D13641 | 285986 |
| D14657 | 285938 |
| L16785 | 349475 |
| D87469 | 1665820 |
| X13709 | 35389 |
| M19961 | 180940 |
| AL049974 | 4884224 |
| M19961 | 180940 |
| X02761 | 31396 |
| Y08982 | 3256190 |
| X96484 | 1223740 |
| AL049339 | 4500122 |
| AF114264 | 4768674 |
| AF114264 | 4768674 |
| Z13009 | 31072 |
| AB002330 | 2224604 |
| AF092128 | 5138905 |
| AF086408 | 3483753 |
| M24486 | 190785 |
| M24486 | 190785 |
| AB020657 | 4240188 |
| D87667 | 1620019 |
| D87667 | 1620019 |
| X15187 | 37260 |
| M81757 | 337732 |
| M38188 | 189378 |
| AJ007669 | 3860407 |
| M64098 | 183891 |
| J02943 | 179970 |
| AB002439 | 2943814 |
| S72481 | 632789 |
| S72481 | 632789 |
| D00099 | 219941 |
| D00099 | 219941 |
| M10905 | 182696 |
| M14060 | 182701 |
| Z31696 | 479156 |
| X81109 | 535057 |
| J03202 | 186916 |
| U46194 | 1517908 |
| AB018281 | 3882196 |
| X59618 | 36154 |
| AF016582 | 2367668 |
| X79535 | 496886 |
| L13806 | 306554 |
| U75283 | 1906590 |
| Y00052 | 30308 |
| M23254 | 511636 |
| X69141 | 435676 |
| J03202 | 186916 |
| K00799 | 182681 |
| AF068706 | 3193225 |
| J02814 | 189875 |
| K00799 | 182681 |
| AB021654 | 4062862 |
| AF016535 | 2353263 |
| M10905 | 182696 |
| AB007862 | 2662084 |
| U46194 | 1517908 |
| AF070550 | 3387913 |
| J03796 | 182072 |
| X04098 | 28338 |
| U30246 | 903681 |
| U25789 | 808089 |
| AF086336 | 3483681 |
| M22918 | 189019 |
| M10905 | 182696 |
| U25766 | 808064 |
| X79535 | 496886 |
| L16785 | 349475 |
| L16785 | 349475 |
| M22636 | 337446 |
| M22636 | 337446 |
| D87735 | 1620021 |
| X07979 | 31441 |
| U05598 | 531159 |
| AB021654 | 4062862 |
| X17206 | 34391 |
| D87735 | 1620021 |
| J04088 | 292829 |
| AF046025 | 4581063 |
| AF046025 | 4581063 |
| AF086336 | 3483681 |
| AJ011007 | 4468340 |
| X02761 | 31396 |
| X02761 | 31396 |
| Y00819 | 37508 |
| X04098 | 28338 |
| X04098 | 28338 |
| AF086205 | 3483550 |
| X85373 | 806565 |
| X85373 | 806565 |
| L12387 | 459835 |
| L12387 | 459835 |
| AF054990 | 3005703 |
| AL049929 | 4884174 |
| K00799 | 182681 |
| U42404 | 4096845 |
| AF086336 | 3483681 |
| AF086336 | 3483681 |
| K00799 | 182681 |
| D29992 | 484050 |
| D29992 | 484050 |
| U31384 | 995920 |
| U31384 | 995920 |
| AF086336 | 3483681 |
| AF086336 | 3483681 |
| AF077043 | 4689133 |
| AF043431 | 3452280 |
| AF043431 | 3452280 |
| M17885 | 190231 |
| U73377 | 1658387 |
| X70394 | 468707 |
| X70394 | 468707 |
| X00351 | 28251 |
| X63432 | 28335 |
| X02308 | 37478 |
| M10905 | 182696 |
| M10905 | 182696 |
| Z26317 | 416177 |
| Z26317 | 416177 |
| M10905 | 182696 |
| M22918 | 189019 |
| AF011793 | 2352903 |
| AJ001309 | 3171907 |
| AF068706 | 3193225 |
| X67731 | 525240 |
| AF016535 | 2353263 |
| AF016535 | 2353263 |
| X02761 | 31396 |
| AB007883 | 2887420 |
| AB011089 | 3043557 |
| L13806 | 306554 |
| M10905 | 182696 |
| L05425 | 179284 |
| L05425 | 179284 |
| D29992 | 484050 |
| AF007791 | 3779196 |
| D83776 | 1228034 |
| D83776 | 1228034 |
| AB014577 | 3327167 |
| AB014577 | 3327167 |
| X55740 | 23896 |
| X55740 | 23896 |
| L05425 | 179284 |
| AB017116 | 4586883 |
| AB017116 | 4586883 |
| K00799 | 182681 |
| U42404 | 4096845 |
| U42404 | 4096845 |
| J03198 | 183224 |
| J03198 | 183224 |
| M58549 | 187592 |
| D29992 | 484050 |
| AB007883 | 2887420 |
| AB007883 | 2887420 |
| M33308 | 340236 |
| M33308 | 340236 |
| D87735 | 1620021 |
| D87735 | 1620021 |
| AB011128 | 3043635 |
| AJ010841 | 3646127 |
| AJ010841 | 3646127 |
| U42404 | 4096845 |
| U42404 | 4096845 |
| M23114 | 184100 |
| S75169 | 896223 |
| S75169 | 896223 |
| X02761 | 31396 |
| AF007791 | 3779196 |
| K00799 | 182681 |
| K00799 | 182681 |
| AF072928 | 3916215 |
| K00799 | 182681 |
| U76421 | 2039299 |
| M58549 | 187592 |
| X02761 | 31396 |
| X02761 | 31396 |
| M69181 | 641957 |
| M69181 | 641957 |
| AB019568 | 3885371 |
| AF031385 | 2606093 |
| X04470 | 28638 |
| X04470 | 28638 |
| U76421 | 2039299 |
| AB023191 | 4589591 |
| AB023191 | 4589591 |
| U34994 | 6525311 |
| U34994 | 6525311 |
| X84407 | 666046 |
| AJ011001 | 4456466 |
| AJ011001 | 4456466 |
| D13748 | 219402 |
| D13748 | 219402 |
| D29992 | 484050 |
| D29992 | 484050 |
| U42457 | 4096851 |
| U42457 | 4096851 |
| AL050282 | 4886464 |
| E01650 | 2169903 |
| J03779 | 179833 |
| K00799 | 182681 |
| K00799 | 182681 |
| AF085844 | 3483158 |
| M16462 | 181556 |
| Y09565 | 2894089 |
| D87667 | 1620019 |
| D87667 | 1620019 |
| L05093 | 401844 |
| AB007883 | 2887420 |
| U14971 | 550022 |
| U14971 | 550022 |
| U44754 | 1174202 |
| U44754 | 1174202 |
| X60656 | 31134 |
| AB018346 | 3882326 |
| U85658 | 2058552 |
| U85658 | 2058552 |
| M69181 | 641957 |
| AF008551 | 2979627 |
| AF008551 | 2979627 |
| D29992 | 484050 |
| S77362 | 944970 |
| J05032 | 179101 |
| AF052178 | 3360489 |
| AF052178 | 3360489 |
| AJ010841 | 3646127 |
| AJ010841 | 3646127 |
| U02032 | 404014 |
| U02032 | 404014 |
| AB011089 | 3043557 |
| AF050638 | 5326819 |
| D85181 | 1906795 |
| AJ010841 | 3646127 |
| AJ010841 | 3646127 |
| U07343 | 463988 |
| U42404 | 4096845 |
| U42404 | 4096845 |
| X04098 | 28338 |
| X04098 | 28338 |
| AF011793 | 2352903 |
| AJ001309 | 3171907 |
| AL049339 | 4500122 |
| AF028832 | 3287488 |
| AF131797 | 4406625 |
| U42404 | 4096845 |
| U42404 | 4096845 |
| X02761 | 31396 |
| AB028974 | 5689438 |
| AB028974 | 5689438 |
| U45976 | 1373145 |
| L22154 | 347385 |
| AF068007 | 3201963 |
| D83776 | 1228034 |
| D83776 | 1228034 |
| J02943 | 179970 |
| J04543 | 338243 |
| M14200 | 181477 |
| U42404 | 4096845 |
| U42404 | 4096845 |
| AF077208 | 4679029 |
| J02943 | 179970 |
| U18062 | 642794 |
| U18062 | 642794 |
| AF010309 | 2754811 |
| AF010309 | 2754811 |
| AL132665 | 6137021 |
| AL132665 | 6137021 |
| X69181 | 505472 |
| J03015 | 337755 |
| J03015 | 337755 |
| X81889 | 1702923 |
| D13641 | 285986 |
| D13641 | 285986 |
| X96484 | 1223740 |
| U42404 | 4096845 |
| X69970 | 32461 |
| J02943 | 179970 |
| M69181 | 641957 |
| J02943 | 179970 |
| D83776 | 1228034 |
| D83776 | 1228034 |
| D87667 | 1620019 |
| M73792 | 179862 |
| X15187 | 37260 |
| X15187 | 37260 |
| AF081280 | 3415120 |
| AF086003 | 3483348 |
| AF086003 | 3483348 |
| M11353 | 184092 |
| M11353 | 184092 |
| M38188 | 189378 |
| D00099 | 219941 |
| M19961 | 180940 |
| M19961 | 180940 |
| E01816 | 2170068 |
| AF100741 | 5138992 |
| AF001176 | 4309675 |
| X04098 | 28338 |
| U92993 | 2781411 |
| A21185 | 579591 |
| D17266 | 598894 |
| AJ002030 | 2570006 |
| AF054179 | 3341999 |
| AF054179 | 3341999 |
| X56932 | 23690 |
| X69970 | 32461 |
| X69970 | 32461 |
| U82130 | 1772663 |
| U82130 | 1772663 |
| M10119 | 182517 |
| M10119 | 182517 |
| L16785 | 349475 |
| AF132959 | 4680688 |
| AF132959 | 4680688 |
| D87667 | 1620019 |
| K00799 | 182681 |
| M55268 | 177837 |
| M55268 | 177837 |
| U42404 | 4096845 |
| X51473 | 31410 |
| AF016535 | 2353263 |
| L10910 | 405191 |
| U42404 | 4096845 |
| U42404 | 4096845 |
| D49737 | 2588778 |
| AF086336 | 3483681 |
| X07897 | 37207 |
| AB011159 | 3043697 |
| AB011159 | 3043697 |
| M81182 | 190128 |
| M81182 | 190128 |
| AF046025 | 4581063 |
| AF046025 | 4581063 |
| AB020649 | 4240172 |
| D21260 | 434760 |
| D21260 | 434760 |
| D14530 | 414348 |
| AF038451 | 3779225 |
| D38255 | 2463543 |
| AF008551 | 2979627 |
| AF008551 | 2979627 |
| AF070648 | 3283922 |
| AF026166 | 4090928 |
| U42404 | 4096845 |
| AF047439 | 3335133 |
| AL021683 | 3217033 |
| J04031 | 187464 |
| M30047 | 187400 |
| Y00819 | 37508 |
| AB014577 | 3327167 |
| AB014577 | 3327167 |
| D29992 | 484050 |
| M11353 | 184092 |
| M11353 | 184092 |
| AF081280 | 3415120 |
| J03202 | 186916 |
| U42404 | 4096845 |
| U42404 | 4096845 |
| U42404 | 4096845 |
| AF086336 | 3483681 |
| AJ011007 | 4468340 |
| M84739 | 179881 |
| M84739 | 179881 |
| AB019568 | 3885371 |
| S80990 | 1911529 |
| AF035812 | 2665835 |
| X02761 | 31396 |
| AF035319 | 2661082 |
| X28433 | notDefined |
| N80412 | 1243113 |
| Q66636 | notDefined |
| V59612 | notDefined |
| T67164 | 676604 |
| V40550 | notDefined |
| V63175 | notDefined |
| V63175 | notDefined |
| X28433 | notDefined |
| V04699 | notDefined |
| V63175 | notDefined |
| V46154 | notDefined |
| X28433 | notDefined |
| V83134 | notDefined |
| Q66636 | notDefined |
| V59663 | notDefined |
| X04408 | 31914 |
| Q70007 | notDefined |
| T15718 | 517880 |
| V89990 | notDefined |
| V32416 | notDefined |
| X28433 | notDefined |
| X28433 | notDefined |
| X40333 | notDefined |
| V59695 | notDefined |
| Q32364 | notDefined |
| Q32364 | notDefined |
| V60015 | notDefined |
| X28433 | notDefined |
| X28433 | notDefined |
| X28433 | notDefined |
| X28433 | notDefined |
| AC10439 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |
| AC13954 | notDefined |

What is claimed is:

1. A method for determining whether TAXOL can be used to reduce the growth of cancer cells, comprising the steps of:
a) obtaining a sample of cancer cells;
b) determining whether the cancer cells express one or more markers identified in Tables 1 and/or 2; and
c) identifying that TAXOL can be used to reduce the growth of said cancer cells when the one or more markers are expressed.

2. The method of claim 1, wherein the level of expression is determined by detecting the amount of mRNA that is encoded by the one or more markers present in the sample.

3. The method of claim 1, wherein the level of expression is determined by detecting the amount of protein that is encoded by said one or more markers present in the sample.

4. The method of claim 1, wherein said cancer cells are obtained from cancer cell lines or cancer cells obtained from a subject.

5. A method for determining whether TAXOL can be used to reduce the growth of cancer cells, comprising the steps of:
a) obtaining a sample of cancer cells;
b) determining whether the cancer cells express one or more markers selected from the group consisting of the sensitivity markers in Table 1; and
c) identifying that TAXOL can be used to reduce the growth of the cancer cells when one or more of the sensitivity markers in Table 1 is expressed by the cancer cells.

6. The method of claim 5, wherein the level of expression is determined by detecting the amount of mRNA that is encoded by the one or more markers present in the sample.

7. The method of claim 5, wherein the level of expression is determined by detecting the amount of protein that is encoded by said one or more markers present in the sample.

8. The method of claim 5, wherein said cancer cells are obtained from cancer cell lines or cancer cells obtained from a subject.

9. A method for determining whether TAXOL cannot be used to reduce the growth of cancer cells, comprising the steps of:
a) obtaining a sample of cancer cells;
b) determining whether the cancer cells express one or more markers selected from the group consisting of the sensitivity markers identified in Table 1; and
identifying that TAXOL cannot be used to reduce the growth of the cancer cells when one or more of the sensitivity markers in Table 1 is not expressed by the cancer cells.

10. The method of claim 9, wherein the level of expression is determined by detecting the amount of mRNA that is encoded by the one or more sensitivity markers present in the sample.

11. The method of claim 9, wherein the level of expression is determined by detecting the amount of protein that is encoded by said one or more markers present in the sample.

12. The method of claim 9, wherein said cancer cells are obtained from cancer cell lines or cancer cells obtained from a subject.

13. A method for determining whether TAXOL can be used to reduce the growth of cancer cells, comprising the steps of:
a) obtaining a sample of cancer cells;
b) determining whether the cancer cells express one or more markers selected from the group consisting of the resistance markers in Table 2; and
c) identifying that TAXOL can be used to reduce the growth of the cancer cells when one or more of the resistance markers in Table 2 is not expressed by the cancer cells.

14. The method of claim 13, wherein the level of expression is determined by detecting the amount of mRNA that is encoded by the one or more markers present in the sample.

15. The method of claim 13, wherein the level of expression is determined by detecting the amount of protein that is encoded by said one or more markers present in the sample.

16. The method of claim 13, wherein said cancer cells are obtained from cancer cell lines or cancer cells obtained from a subject.

17. A method for determining whether TAXOL cannot be used to reduce the growth of cancer cells, comprising the steps of:
a) obtaining a sample of cancer cells;
b) determining whether the cancer cells express one or more markers selected from the group consisting of the resistance markers identified in Table 2; and
c) identifying that TAXOL cannot be used to reduce the growth of the cancer cells when one or more of the markers in Table 2 is expressed by the cancer cells.

18. The method of claim 17, wherein the level of expression is determined by detecting the amount of mRNA that is encoded by the one or more markers present in the sample.

19. The method of claim 17, wherein the level of expression is determined by detecting the amount of protein that is encoded by said one or more markers present in the sample.

20. The method of claim 17, wherein the cancer cells are obtained from cancer cell lines or cancer cells obtained from a subject.

21. A method for determining whether TAXOL can be used to reduce the growth of cancer cells, comprising the steps of:
a) obtaining a sample of cancer cells;
b) exposing the cancer cell to one or more test agents;
c) determining the level of expression in the cancer cells of one or more markers selected from the group consisting of the sensitivity markers identified in Tables 1 in the sample exposed to the agent and in a sample of cancer cells that is not exposed to the agent; and
d) identifying that TAXOL can be used to reduce the growth of said cancer cells when the expression of one or more of said markers is increased in the presence of said agent.

22. The method of claim 21, wherein the level of expression is determined by detecting the amount of mRNA that is encoded by the one or more markers present in the sample.

23. The method of claim 21, wherein the level of expression is determined by detecting the amount of protein that is encoded by said one or more markers present in the sample.

24. The method of claim 21, wherein the cancer cells are obtained from cancer cell lines or cancer cells obtained from a subject.

25. A method for determining whether TAXOL cannot be used to reduce the growth of cancer cells, comprising the steps of:
a) obtaining a sample of cancer cells;
b) exposing the cancer cell to one or more test agents;
c) determining the level of expression in the cancer cells of one or more markers selected from the group consisting of the sensitivity markers identified in Tables 1 in the sample exposed to the agent and in a sample of cancer cells that is not exposed to the agent; and
d) identifying that TAXOL cannot be used to reduce the growth of the cancer cells when the expression of one or more of said markers is not increased in the presence of said agent.

26. The method of claim 25, wherein the level of expression is determined by detecting the amount of mRNA that is encoded by the one or more markers present in the sample.

27. The method of claim 25, wherein the level of expression is determined by detecting the amount of protein that is encoded by said one or more markers present in the sample.

28. The method of claim 25, wherein the cancer cells are obtained from cancer cell lines or cancer cells obtained from a subject.

29. A method for determining whether TAXOL can be used to reduce the growth of cancer cells, comprising the steps of:
a) obtaining a sample of cancer cells;
b) exposing the cancer cell to one or more test agents;
c) determining the level of expression in the cancer cells of one or more markers selected from the group consisting of the resistance markers identified in Table 2 in the sample exposed to the agent and in a sample of cancer cells that is not exposed to the agent; and
d) identifying that TAXOL can be used to reduce the growth of the cancer cells when the expression of one or more of said markers is not increased in the presence of said agent.

30. The method of claim 29, wherein the level of expression is determined by detecting the amount of mRNA that is encoded by the one or more markers present in the sample.

31. The method of claim 29, wherein the level of expression is determined by detecting the amount of protein that is encoded by said one or more markers present in the sample.

32. The method of claim 29, wherein the cancer cells are obtained from cancer cell lines or cancer cells obtained from a subject.

33. A method for determining whether TAXOL cannot be used to reduce the growth of cancer cells, comprising the steps of:
a) obtaining a sample of cancer cells;
b) exposing the cancer cell to one or more test agents;
c) determining the level of expression in the cancer cells of one or more markers selected from the group consisting of the resistance markers identified in Table 2 in the sample exposed to the agent and in a sample of cancer cells that is not exposed to the agent; and
d) identifying that TAXOL can be used to reduce the growth of the cancer cells when the expression of one or more of said markers is increased in the presence of said agent.

34. The method of claim 33, wherein the level of expression is determined by detecting the amount of mRNA that is encoded by the one or more markers present in the sample.

35. The method of claim 33, wherein the level of expression is determined by detecting the amount of protein that is encoded by said one or more markers present in the sample.

36. The method of claim 33, wherein the cancer cells are obtained from cancer cell lines or cancer cells obtained from a subject.

37. A method for determining whether treatment with TAXOL should be continued in a cancer patient, comprising the steps of:
a) obtaining two or more samples comprising cancer cells from a patient during the course of TAXOL treatment;
b) determining the level of expression in the cancer cells of one or more markers selected from the group consisting of the sensitivity markers identified in Table 1 in the two or more samples; and
c) continuing treatment when the expression level of one or more of the markers does not decrease during the course of treatment.

38. The method of claim 37, wherein the level of expression is determined by detecting the amount of mRNA that is encoded by the one or more markers present in the sample.

39. The method of claim 37, wherein the level of expression is determined by detecting the amount of protein that is encoded by said one or more markers present in the sample.

40. A method for determining whether treatment with TAXOL should not be continued in a cancer patient, comprising the steps of:
a) obtaining two or more samples comprising cancer cells from a patient during the course of TAXOL treatment;
b) determining the level of expression in the cancer cells of one or more markers selected from the group consisting of the sensitivity markers identified in Table 1 in the two or more samples; and
c) continuing treatment when the expression level of one or more of the markers decreases during the course of treatment.

41. The method of claim 40, wherein the level of expression is determined by detecting the amount of mRNA that is encoded by the one or more markers present in the sample.

42. The method of claim 40, wherein the level of expression is determined by detecting the amount of protein that is encoded by said one or more markers present in the sample.

43. A method for determining whether treatment with TAXOL should not be continued in a cancer patient, comprising the steps of:
a) obtaining two or more samples comprising cancer cells from a patient during the course of TAXOL treatment;
b) determining the level of expression in the cancer cells of one or more markers selected from the group consisting of the resistance markers identified in Table 2 in the two or more samples; and
c) discontinuing treatment when the expression level of one or more of the markers does not decrease during the course of treatment.

44. The method of claim 43, wherein the level of expression is determined by detecting the amount of mRNA that is encoded by the one or more markers present in the sample.

45. The method of claim 43, wherein the level of expression is determined by detecting the amount of protein that is encoded by said one or more markers present in the sample.

46. A method for determining whether treatment with TAXOL should be continued in a cancer patient, comprising the steps of:
a) obtaining two or more samples comprising cancer cells from a patient during the course of TAXOL treatment;
b) determining the level of expression in the cancer cells of one or more markers selected from the group consisting of the resistance markers identified in Table 2 in the two or more samples; and
c) continuing treatment when the expression level of one or more of the markers does not increase during the course of treatment.

47. The method of claim 46, wherein the level of expression is determined by detecting the amount of mRNA that is encoded by the one or more markers present in the sample.

48. The method of claim 46, wherein the level of expression is determined by detecting the amount of protein that is encoded by said one or more markers present in the sample.
